SEQUENCE LISTING (40 sequences)

SEQ ID NO: 1 - protein, 2944 aa, Homo sapiens
MTLRLLVAALCAGILAEAPRVRAQHRERVTCTRLYAADIVFLLDGSSSIGRSNFREVRSFLEGLVLPFSGAASAQGVRFATVQYSDDPRTEFGLDALGSGGDVIRAIRELSYKGGNTRTGAAILHVADHVFLPQLARPGVPKVCILITDGKSQDLVDTAAQRLKGQGVKLFAVGIKNADPEELKRVASQPTSDFFFFVNDFSILRTLLPLVSRRVCTTAGGVPVTRPPDDSTSAPRDLVLSEPSSQSLRVQWTAASGPVTGYKVQYTPLTGLGQPLPSERQEVNVPAGETSVRLRGLRPLTEYQVTVIALYANSIGEAVSGTARTTALEGPELTIQNTTAHSLLVAWRSVPGATGYRVTWRVLSGGPTQQQELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATELPGQRVRVSWSPVPGATQYRIIVRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQKGEPGEMGLRGQVGPPGDPGLPGRTGAPGPQGPPGSATAKGERGFPGADGRPGSPGRAGNPGTPGAPGLKGSPGLPGPRGDPGERGPRGPKGEPGAPGQVIGGEGPGLPGRKGDPGPSGPPGPRGPLGDPGPRGPPGLPGTAMKGDKGDRGERGPPGPGEGGIAPGEPGLPGLPGSPGPQGPVGPPGKKGEKGDSEDGAPGLPGQPGSPGEQGPRGPPGAIGPKGDRGFPGPLGEAGEKGERGPPGPAGSRGLPGVAGRPGAKGPEGPPGPTGRQGEKGEPGRPGDPAVVGPAVAGPKGEKGDVGPAGPRGATGVQGERGPPGLVLPGDPGPKGDPGDRGPIGLTGRAGPPGDSGPPGEKGDPGRPGPPGPVGPRGRDGEVGEKGDEGPPGDPGLPGKAGERGLRGAPGVRGPVGEKGDQGDPGEDGRNGSPGSSGPKGDRGEPGPPGPPGRLVDTGPGAREKGEPGDRGQEGPRGPKGDPGLPGAPGERGIEGFRGPPGPQGDPGVRGPAGEKGDRGPPGLDGRSGLDGKPGAAGPSGPNGAAGKAGDPGRDGLPGLRGEQGLPGPSGPPGLPGKPGEDGKPGLNGKNGEPGDPGEDGRKGEKGDSGASGREGRDGPKGERGAPGILGPQGPPGLPGPVGPPGQGFPGVPGGTGPKGDRGETGSKGEQGLPGERGLRGEPGSVPNVDRLLETAGIKASALREIVETWDESSGSFLPVPERRRGPKGDSGEQGPPGKEGPIGFPGERGLKGDRGDPGPQGPPGLALGERGPPGPSGLAGEPGKPGIPGLPGRAGGVGEAGRPGERGERGEKGERGEQGRDGPPGLPGTPGPPGPPGPKVSVDEPGPGLSGEQGPPGLKGAKGEPGSNGDQGPKGDRGVPGIKGDRGEPGPRGQDGNPGLPGERGMAGPEGKPGLQGPRGPPGPVGGHGDPGPPGAPGLAGPAGPQGPSGLKGEPGETGPPGRGLTGPTGAVGLPGPPGPSGLVGPQGSPGLPGQVGETGKPGAPGRDGASGKDGDRGSPGVPGSPGLPGPVGPKGEPGPTGAPGQAVVGLPGAKGEKGAPGGLAGDLVGEPGAKGDRGLPGPRGEKGEAGRAGEPGDPGEDGQKGAPGPKGFKGDPGVGVPGSPGPPGPPGVKGDLGLPGLPGAPGVVGFPGQTGPRGEMGQPGPSGERGLAGPPGREGIPGPLGPPGPPGSVGPPGASGLKGDKGDPGVGLPGPRGERGEPGIRGEDGRPGQEGPRGLTGPPGSRGERGEKGDVGSAGLKGDKGDSAVILGPPGPRGAKGDMGERGPRGLDGDKGPRGDNGDPGDKGSKGEPGDKGSAGLPGLRGLLGPQGQPGAAGIPGDPGSPGKDGVPGIRGEKGDVGFMGPRGLKGERGVKGACGLDGEKGDKGEAGPPGRPGLAGHKGEMGEPGVPGQSGAPGKEGLIGPKGDRGFDGQPGPKGDQGEKGERGTPGIGGFPGPSGNDGSAGPPGPPGSVGPRGPEGLQGQKGERGPPGERVVGAPGVPGAPGERGEQGRPGPAGPRGEKGEAALTEDDIRGFVRQEMSQHCACQGQFIASGSRPLPSYAADTAGSQLHAVPVLRVSHAEEEERVPPEDDEYSEYSEYSVEEYQDPEAPWDSDDPCSLPLDEGSCTAYTLRWYHRAVTGSTEACHPFVYGGCGGNANRFGTREACERRCPPRVVQSQGTGTAQD

SEQ ID NO: 2 - DNA, 9169 bp, Homo sapiens
  source 1..9169: mol_type = genomic DNA
gatgacgctgcggcttctggtggccgcgctctgcgccgggatcctggcagaggcgccccgagtgcgagcccagcacagggagagagtgacctgcacgcgcctttacgccgctgacattgtgttcttactggatggctcctcatccattggccgcagcaatttccgcgaggtccgcagctttctcgaagggctggtgctgcctttctctggagcagccagtgcacagggtgtgcgctttgccacagtgcagtacagcgatgacccacggacagagttcggcctggatgcacttggctctgggggtgatgtgatccgcgccatccgtgagcttagctacaaggggggcaacactcgcacaggggctgcaattctccatgtggctgaccatgtcttcctgccccagctggcccgacctggtgtccccaaggtctgcatcctgatcacagacgggaagtcccaggacctggtggacacagctgcccaaaggctgaaggggcagggggtcaagctatttgctgtggggatcaagaatgctgaccctgaggagctgaagcgagttgcctcacagcccaccagtgacttcttcttcttcgtcaatgacttcagcatcttgaggacactactgcccctcgtttcccggagagtgtgcacgactgctggtggcgtgcctgtgacccgacctccggatgactcgacctctgctccacgagacctggtgctgtctgagccaagcagccaatccttgagagtacagtggacagcggccagtggccctgtgactggctacaaggtccagtacactcctctgacggggctgggacagccactgccgagtgagcggcaggaggtgaacgtcccagctggtgagaccagtgtgcggctgcggggtctccggccactgaccgagtaccaagtgactgtgattgccctctacgccaacagcatcggggaggctgtgagcgggacagctcggaccactgccctagaagggccggaactgaccatccagaataccacagcccacagcctcctggtggcctggcggagtgtgccaggtgccactggctaccgtgtgacatggcgggtcctcagtggtgggcccacacagcagcaggagctgggccctgggcagggttcagtgttgctgcgtgacttggagcctggcacggactatgaggtgaccgtgagcaccctatttggccgcagtgtggggcccgccacttccctgatggctcgcactgacgcttctgttgagcagaccctgcgcccggtcatcctgggccccacatccatcctcctttcctggaacttggtgcctgaggcccgtggctaccggttggaatggcggcgtgagactggcttggagccaccgcagaaggtggtactgccctctgatgtgacccgctaccagttggatgggctgcagccgggcactgagtaccgcctcacactctacactctgctggagggccacgaggtggccacccctgcaaccgtggttcccactggaccagagctgcctgtgagccctgtaacagacctgcaagccaccgagctgcccgggcagcgggtgcgagtgtcctggagcccagtccctggtgccacccagtaccgcatcattgtgcgcagcacccagggggttgagcggaccctggtgcttcctgggagtcagacagcattcgacttggatgacgttcaggctgggcttagctacactgtgcgggtgtctgctcgagtgggtccccgtgagggcagtgccagtgtcctcactgtccgccgggagccggaaactccacttgctgttccagggctgcgggttgtggtgtcagatgcaacgcgagtgagggtggcctggggacccgtccctggagccagtggatttcggattagctggagcacaggcagtggtccggagtccagccagacactgcccccagactctactgccacagacatcacagggctgcagcctggaaccacctaccaggtggctgtgtcggtactgcgaggcagagaggagggccctgctgcagtcatcgtggctcgaacggacccactgggcccagtgaggacggtccatgtgactcaggccagcagctcatctgtcaccattacctggaccagggttcctggcgccacaggatacagggtttcctggcactcagcccacggcccagagaaatcccagttggtttctggggaggccacggtggctgagctggatggactggagccagatactgagtatacggtgcatgtgagggcccatgtggctggcgtggatgggccccctgcctctgtggttgtgaggactgcccctgagcctgtgggtcgtgtgtcgaggctgcagatcctcaatgcttccagcgacgttctacggatcacctgggtaggggtcactggagccacagcttacagactggcctggggccggagtgaaggcggccccatgaggcaccagatactcccaggaaacacagactctgcagagatccggggtctcgaaggtggagtcagctactcagtgcgagtgactgcacttgtcggggaccgcgagggcacacctgtctccattgttgtcactacgccgcctgaggctccgccagccctggggacgcttcacgtggtgcagcgcggggagcactcgctgaggctgcgctgggagccggtgcccagagcgcagggcttccttctgcactggcaacctgagggtggccaggaacagtcccgggtcctggggcccgagctcagcagctatcacctggacgggctggagccagcgacacagtaccgcgtgaggctgagtgtcctagggccagctggagaagggccctctgcagaggtgactgcgcgcactgagtcacctcgtgttccaagcattgaactacgtgtggtggacacctcgatcgactcggtgactttggcctggactccagtgtccagggcatccagctacatcctatcctggcggccactcagaggccctggccaggaagtgcctgggtccccgcagacacttccagggatctcaagctcccagcgggtgacagggctagagcctggcgtctcttacatcttctccctgacgcctgtcctggatggtgtgcggggtcctgaggcatctgtcacacagacgccagtgtgcccccgtggcctggcggatgtggtgttcctaccacatgccactcaagacaatgctcaccgtgcggaggctacgaggagggtcctggagcgtctggtgttggcacttgggcctcttgggccacaggcagttcaggttggcctgctgtcttacagtcatcggccctccccactgttcccactgaatggctcccatgaccttggcattatcttgcaaaggatccgtgacatgccctacatggacccaagtgggaacaacctgggcacagccgtggtcacagctcacagatacatgttggcaccagatgctcctgggcgccgccagcacgtaccaggggtgatggttctgctagtggatgaacccttgagaggtgacatattcagccccatccgtgaggcccaggcttctgggcttaatgtggtgatgttgggaatggctggagcggacccagagcagctgcgtcgcttggcgccgggtatggactctgtccagaccttcttcgccgtggatgatgggccaagcctggaccaggcagtcagtggtctggccacagccctgtgtcaggcatccttcactactcagccccggccagagccctgcccagtgtattgtccaaagggccagaagggggaacctggagagatgggcctgagaggacaagttgggcctcctggcgaccctggcctcccgggcaggaccggtgctcccggcccccaggggccccctggaagtgccactgccaagggcgagaggggcttccctggagcagatgggcgtccaggcagccctggccgcgccgggaatcctgggacccctggagcccctggcctaaagggctctccagggttgcctggccctcgtggggacccgggagagcgaggacctcgaggcccaaagggggagccgggggctcccggacaagtcatcggaggtgaaggacctgggcttcctgggcggaaaggggaccctggaccatcgggcccccctggacctcgtggaccactgggggacccaggaccccgtggccccccagggcttcctggaacagccatgaagggtgacaaaggcgatcgtggggagcggggtccccctggaccaggtgaaggtggcattgctcctggggagcctgggctgccgggtcttcccggaagccctggaccccaaggccccgttggcccccctggaaagaaaggagaaaaaggtgactctgaggatggagctccaggcctcccaggacaacctgggtctccgggtgagcagggcccacggggacctcctggagctattggccccaaaggtgaccggggctttccagggcccctgggtgaggctggagagaagggcgaacgtggacccccaggcccagcgggatcccgggggctgccaggggttgctggacgtcctggagccaagggtcctgaagggccaccaggacccactggccgccaaggagagaagggggagcctggtcgccctggggaccctgcagtggtgggacctgctgttgctggacccaaaggagaaaagggagatgtggggcccgctgggcccagaggagctaccggagtccaaggggaacggggcccacccggcttggttcttcctggagaccctggccccaagggagaccctggagaccggggtcccattggccttactggcagagcaggacccccaggtgactcagggcctcctggagagaagggagaccctgggcggcctggccccccaggacctgttggcccccgaggacgagatggtgaagttggagagaaaggtgacgagggtcctccgggtgacccgggtttgcctggaaaagcaggcgagcgtggccttcggggggcacctggagttcgggggcctgtgggtgaaaagggagaccagggagatcctggagaggatggacgaaatggcagccctggatcatctggacccaagggtgaccgtggggagccgggtcccccaggacccccgggacggctggtagacacaggacctggagccagagagaagggagagcctggggaccgcggacaagagggtcctcgagggcccaagggtgatcctggcctccctggagcccctggggaaaggggcattgaagggtttcggggacccccaggcccacagggggacccaggtgtccgaggcccagcaggagaaaagggtgaccggggtccccctgggctggatggccggagcggactggatgggaaaccaggagccgctgggccctctgggccgaatggtgctgcaggcaaagctggggacccagggagagacgggcttccaggcctccgtggagaacagggcctccctggcccctctggtccccctggattaccgggaaagccaggcgaggatggcaaacctggcctgaatggaaaaaacggagaacctggggaccctggagaagacgggaggaagggagagaaaggagattcaggcgcctctgggagagaaggtcgtgatggccccaagggtgagcgtggagctcctggtatccttggaccccaggggcctccaggcctcccagggccagtgggccctcctggccagggttttcctggtgtcccaggaggcacgggccccaagggtgaccgtggggagactggatccaaaggggagcagggcctccctggagagcgtggcctgcgaggagagcctggaagtgtgccgaatgtggatcggttgctggaaactgctggcatcaaggcatctgccctgcgggagatcgtggagacctgggatgagagctctggtagcttcctgcctgtgcccgaacggcgtcgaggccccaagggggactcaggcgaacagggccccccaggcaaggagggccccatcggctttcctggagaacgcgggctgaagggcgaccgtggagaccctggccctcaggggccacctggtctggcccttggggagaggggcccccccgggccttccggccttgccggggagcctggaaagcctggtattcccgggctcccaggcagggctgggggtgtgggagaggcaggaaggccaggagagaggggagaacggggagagaaaggagaacgtggagaacagggcagagatggccctcctggactccctggaacccctgggccccccggaccccctggccccaaggtgtctgtggatgagccaggtcctggactctctggagaacagggaccccctggactcaagggtgctaagggggagccgggcagcaatggtgaccaaggtcccaaaggagacaggggtgtgccaggcatcaaaggagaccggggagagcctggaccgaggggtcaggacggcaacccgggtctaccaggagagcgtggtatggctgggcctgaagggaagccgggtctgcagggtccaagaggcccccctggcccagtgggtggtcatggagaccctggaccacctggtgccccgggtcttgctggccctgcaggaccccaaggaccttctggcctgaagggggagcctggagagacaggacctccaggacggggcctgactggacctactggagctgtgggacttcctggaccccccggcccttcaggccttgtgggtccacaggggtctccaggtttgcctggacaagtgggggagacagggaagccgggagccccaggtcgagatggtgccagtggaaaagatggagacagagggagccctggtgtgccagggtcaccaggtctgcctggccctgtcggacctaaaggagaacctggccccacgggggcccctggacaggctgtggtcgggctccctggagcaaagggagagaagggagcccctggaggccttgctggagacctggtgggtgagccgggagccaaaggtgaccgaggactgccagggccgcgaggcgagaagggtgaagctggccgtgcaggggagcccggagaccctggggaagatggtcagaaaggggctccaggacccaaaggtttcaagggtgacccaggagtcggggtcccgggctcccctgggcctcctggccctccaggtgtgaagggagatctgggcctccctggcctgcccggtgctcctggtgttgttgggttcccgggtcagacaggccctcgaggagagatgggtcagccaggccctagtggagagcggggtctggcaggccccccagggagagaaggaatcccaggacccctggggccacctggaccaccggggtcagtgggaccacctggggcctctggactcaaaggagacaagggagaccctggagtagggctgcctgggccccgaggcgagcgtggggagccaggcatccggggtgaagatggccgccccggccaggagggaccccgaggactcacggggccccctggcagcaggggagagcgtggggagaagggtgatgttgggagtgcaggactaaagggtgacaagggagactcagctgtgatcctggggcctccaggcccacggggtgccaagggggacatgggtgaacgagggcctcggggcttggatggtgacaaaggacctcggggagacaatggggaccctggtgacaagggcagcaagggagagcctggtgacaagggctcagccgggttgccaggactgcgtggactcctgggaccccagggtcaacctggtgcagcagggatccctggtgacccgggatccccaggaaaggatggagtgcctggtatccgaggagaaaaaggagatgttggcttcatgggtccccggggcctcaagggtgaacggggagtgaagggagcctgtggccttgatggagagaagggagacaagggagaagctggtcccccaggccgccccgggctggcaggacacaaaggagagatgggggagcctggtgtgccgggccagtcgggggcccctggcaaggagggcctgatcggtcccaagggtgaccgaggctttgacgggcagccaggccccaagggtgaccagggcgagaaaggggagcggggaaccccaggaattgggggcttcccaggccccagtggaaatgatggctctgctggtcccccagggccacctggcagtgttggtcccagaggccccgaaggacttcagggccagaagggtgagcgaggtccccccggagagagagtggtgggggctcctggggtccctggagctcctggcgagagaggggagcaggggcggccagggcctgccggtcctcgaggcgagaagggagaagctgcactgacggaggatgacatccggggctttgtgcgccaagagatgagtcagcactgtgcctgccagggccagttcatcgcatctggatcacgacccctccctagttatgctgcagacactgccggctcccagctccatgctgtgcctgtgctccgcgtctctcatgcagaggaggaagagcgggtaccccctgaggatgatgagtactctgaatactccgagtattctgtggaggagtaccaggaccctgaagctccttgggatagtgatgacccctgttccctgccactggatgagggctcctgcactgcctacaccctgcgctggtaccatcgggctgtgacaggcagcacagaggcctgtcacccttttgtctatggtggctgtggagggaatgccaaccgttttgggacccgtgaggcctgcgagcgccgctgcccaccccgggtggtccagagccaggggacaggtactgcccaggactgaggcccagataatgagctgagattcagcatcccctggaggagtcggggtctcagcagaaccccactgtccctccccttggtgctagaggcttgtgtgcacgtgagcgtgcgtgtgcacgtccgttatttcagtgacttggtcccgtgggtctagccttcccccctgtggacaaacccccattgtggctcctgccaccctggcagatgactcactgtgggggggtggctgtgggcagtgagcggatgtgactggcgtctgacccgccccttgacccaagcctgtgatgacatggtgctgattctggggggcattaaagctgctgttttaaaaggc

SEQ ID NO: 3 - protein, 493 aa, Homo sapiens
MAAATGPSFWLGNETLKVPLALFALNRQRLCERLRKNPAVQAGSIVVLQGGEETQRYCTDTGVLFRQESFFHWAFGVTEPGCYGVIDVDTGKSTLFVPRLPASHATWMGKIHSKEHFKEKYAVDDVQYVDEIASVLTSQKPSVLLTLRGVNTDSGSVCREASFDGISKFEVNNTILHPEIVECRVFKTDMELEVLRYTNKISSEAHREVMKAVKVGMKEYELESLFEHYCYSRGGMRHSSYTCICGSGENSAVLHYGHAGAPNDRTIQNGDMCLFDMGGEYYCFASDITCSFPANGKFTADQKAVYEAVLRSSRAVMGAMKPGVWWPDMHRLADRIHLEELAHMGILSGSVDAMVQAHLGAVFMPHGLGHFLGIDVHDVGGYPEGVERIDEPGLRSLRTARHLQPGMVLTVEPGIYFIDHLLDEALADPARASFLNREVLQRFRGFGGVRIEEDVVVTDSGIELLTCVPRTVEEIEACMAGCDKAFTPFSGPK

SEQ ID NO: 4 - DNA, 2019 bp, Homo sapiens
  source 1..2019: mol_type = genomic DNA
atttggctctggaggaggtgggtcaaggactccatgccccgccccctgccccgcccttaccccgccccatgcattggcacccggaggggctcagctgacgccgcacttcacgtgacgccggtgccgggcgaacatggcggcggccaccggaccctcgttttggctggggaatgaaaccctgaaggtgccgctggcgctctttgccttgaaccggcagcgcctgtgtgagcggctgcggaagaaccctgctgtgcaggccggctccatcgtggtcctgcagggcggggaggagactcagcgctactgcaccgacaccggggtcctcttccgccaggagtccttctttcactgggcgttcggtgtcactgagccaggctgctatggtgtcatcgatgttgacactgggaagtcgaccctgtttgtgcccaggcttcctgccagccatgccacctggatgggaaagatccattccaaggagcacttcaaggagaagtatgccgtggacgacgtccagtacgtagatgagattgccagcgtcctgacgtcacagaagccctctgtcctcctcactttgcgtggcgtcaacacggacagcggcagtgtctgcagggaggcctcctttgacggcatcagcaagttcgaagtcaacaataccattcttcacccagagatcgttgagtgccgagtgtttaagacggatatggagctggaggttctgcgctataccaataaaatctccagcgaggcccaccgtgaggtaatgaaggctgtaaaagtgggaatgaaagaatatgagttggaaagcctcttcgagcactactgctactcccggggcggcatgcgccacagctcctacacctgcatctgcggcagtggtgagaactcagccgtgctacactacggacacgccggagctcccaacgaccgaacgatccagaatggggatatgtgcctgttcgacatgggcggtgagtattactgcttcgcttccgacatcacctgctcctttcccgccaacggcaagttcactgcagaccagaaggccgtctatgaggcagtgctgcggagctcccgtgccgtcatgggtgccatgaagccaggtgtctggtggcctgacatgcaccgcctggctgaccgcatccacctggaggagctggcccacatgggcatcctgagcggcagcgtggacgccatggtccaggctcacctgggggccgtgtttatgcctcacgggcttggccacttcctgggcattgacgtgcacgacgtgggaggctacccagagggcgtggagcgcatcgacgagcccggcctgcggagcctgcgcactgcacggcacctgcagccaggcatggtgctcaccgtggagccgggcatctacttcatcgaccacctcctggatgaggccctggcggacccggcccgcgcctccttccttaaccgcgaggtcctgcagcgctttcgcggttttggcggggtccgcatcgaggaggacgtcgtggtgactgacagcggcatagagctgctgacctgcgtgccccgcactgtggaagagattgaagcatgcatggcaggctgtgacaaggcctttacccccttctctggccccaagtagagccagccagaaatcccagcgcacctgggggcctggccttgcaacctcttttcgtgatgggcagcctgctggtcagcactccagtagcgagagacggcacccagaatcagatcccagcttcggcatttgatcagaccaaacagtgctgtttcccggggaggaaacacttttttaattacccttttgcaggctcccacctttaatctgttttataccttgcttattaaatgagcgacttaaaatgattgaaaataatgctgttctttagtagcaactaaaatgtgtcttgctgtcatttatattccttttcccaggaaagaagcatttctgatactttctgtcaaaaatcaatatgcagaatggcatttgcaataaaaggtttcctaaaatggtccaaaaaaaaa

SEQ ID NO: 5 - protein, 534 aa, Homo sapiens
MIWYILIIGILLPQSLAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSASDDQSDQKTTPKKKGVAVDYLPERQKYEMLCRGEGIKMTPRRQKKLFCRYHDGNRNPKFILAPAKQEDEWDKPRIIRFHDIISDAEIEIVKDLAKPRLSRATVHDPETGKLTTAQYRVSKSAWLSGYENPVVSRINMRIQDLTGLDVSTAEELQVANYGVGGQYEPHFDFARKDEPDAFKELGTGNRIATWLFYMSDVSAGGATVFPEVGASVWPKKGTAVFWYNLFASGEGDYSTRHAACPVLVGNKWVSNKWLHERGQEFRRPCTLSELE

SEQ ID NO: 6 - DNA, 2860 bp, Homo sapiens
  source 1..2860: mol_type = genomic DNA
ggggcgctggtgtgatcgagctcacgtagcgagggctgcagtcgcctcctccctggcgctgccatcgcggcctagaggttataaaagggctaacgggctccctctgctgcccagtcgcgccgccagcgggctgagggtaggaagtagccgctccgagtggaggcgactgggggctgaagagcgcgccgccctctcgtcccactttccaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgagccgagctacagtacatgaccctgagactggaaaattgaccacagcacagtacagagtatctaagagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgacaaacaggcttccctttttctcctattgttgtactcttatgtgtctgatatacacatttcctagtcttaactttcaggagtttacaattgactaacactccatgattgattcagtcatgaacctcatcccatgtttcatctgtggacaattgcttactttgtgggttcttttaaaagtaacacgaaatcatcatattgcataaaaccttaaagttctgttggtatcacagaagacaaggcagagtttaaagtgaggaattttatatttaaagaactttttggttggataaaaacataatttgagcatccagttttagtatttcactacatctcagttggtgggtgttaagctagaatgggctgtgtgataggaaacaaatgccttacagatgtgcctaggtgttctgtttacctagtgtcttactctgttttctggatctgaagactagtaataaactaggacactaactgggttccatgtgattgccctttcatatgatcttctaagttgatttttttcctcccaagtcttttttaaagaaagtatactgtattttaccaaccccctctcttttcttttagctcctctgtggtgaattaaacgtacttgagttaaaatatttcgattttttttttttttttaatggaaagtcctgcataacaacactgggccttcttaactaaaatgctcaccacttagcctgtttttttatcccttttttaaaatgacagatgattttgttcaggaattttgctgtttttcttagtgctaataccttgcctcttattcctgctacagcagggtggtaatattggcattctgattaaatactgtgccttaggagactggaagtttaaaaatgtacaagtcctttcagtgatgagggaattgattttttttaaaagtctttttcttagaaagccaaaatgtttgtttttttaagattctgaaatgtgttgtgacaacaatgacctatttatgatcttaaatcttttttaaaaaaaaaaaaaaaa

SEQ ID NO: 7 - protein, 534 aa, Homo sapiens
MIWYILIIGILLPQSLAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSASDDQSDQKTTPKKKGVAVDYLPERQKYEMLCRGEGIKMTPRRQKKLFCRYHDGNRNPKFILAPAKQEDEWDKPRIIRFHDIISDAEIEIVKDLAKPRLRRATISNPITGDLETVHYRISKSAWLSGYENPVVSRINMRIQDLTGLDVSTAEELQVANYGVGGQYEPHFDFARKDEPDAFKELGTGNRIATWLFYMSDVSAGGATVFPEVGASVWPKKGTAVFWYNLFASGEGDYSTRHAACPVLVGNKWVSNKWLHERGQEFRRPCTLSELE

SEQ ID NO: 8 - DNA, 2860 bp, Homo sapiens
  source 1..2860: mol_type = genomic DNA
ggggcgctggtgtgatcgagctcacgtagcgagggctgcagtcgcctcctccctggcgctgccatcgcggcctagaggttataaaagggctaacgggctccctctgctgcccagtcgcgccgccagcgggctgagggtaggaagtagccgctccgagtggaggcgactgggggctgaagagcgcgccgccctctcgtcccactttccaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgaggcgagccaccatttcaaacccaataacaggagacttggagacggtacattacagaattagcaaaagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgacaaacaggcttccctttttctcctattgttgtactcttatgtgtctgatatacacatttcctagtcttaactttcaggagtttacaattgactaacactccatgattgattcagtcatgaacctcatcccatgtttcatctgtggacaattgcttactttgtgggttcttttaaaagtaacacgaaatcatcatattgcataaaaccttaaagttctgttggtatcacagaagacaaggcagagtttaaagtgaggaattttatatttaaagaactttttggttggataaaaacataatttgagcatccagttttagtatttcactacatctcagttggtgggtgttaagctagaatgggctgtgtgataggaaacaaatgccttacagatgtgcctaggtgttctgtttacctagtgtcttactctgttttctggatctgaagactagtaataaactaggacactaactgggttccatgtgattgccctttcatatgatcttctaagttgatttttttcctcccaagtcttttttaaagaaagtatactgtattttaccaaccccctctcttttcttttagctcctctgtggtgaattaaacgtacttgagttaaaatatttcgattttttttttttttttaatggaaagtcctgcataacaacactgggccttcttaactaaaatgctcaccacttagcctgtttttttatcccttttttaaaatgacagatgattttgttcaggaattttgctgtttttcttagtgctaataccttgcctcttattcctgctacagcagggtggtaatattggcattctgattaaatactgtgccttaggagactggaagtttaaaaatgtacaagtcctttcagtgatgagggaattgattttttttaaaagtctttttcttagaaagccaaaatgtttgtttttttaagattctgaaatgtgttgtgacaacaatgacctatttatgatcttaaatcttttttaaaaaaaaaaaaaaaa

SEQ ID NO: 9 - protein, 516 aa, Homo sapiens
MIWYILIIGILLPQSLAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSASDDQSDQKTTPKKKGVAVDYLPERQKYEMLCRGEGIKMTPRRQKKLFCRYHDGNRNPKFILAPAKQEDEWDKPRIIRFHDIISDAEIEIVKDLAKPRLSRATVHDPETGKLTTAQYRVSKSAWLSGYENPVVSRINMRIQDLTGLDVSTAEELQKDEPDAFKELGTGNRIATWLFYMSDVSAGGATVFPEVGASVWPKKGTAVFWYNLFASGEGDYSTRHAACPVLVGNKWVSNKWLHERGQEFRRPCTLSELE

SEQ ID NO: 10 - DNA, 2806 bp, Homo sapiens
  source 1..2806: mol_type = genomic DNA
ggggcgctggtgtgatcgagctcacgtagcgagggctgcagtcgcctcctccctggcgctgccatcgcggcctagaggttataaaagggctaacgggctccctctgctgcccagtcgcgccgccagcgggctgagggtaggaagtagccgctccgagtggaggcgactgggggctgaagagcgcgccgccctctcgtcccactttccaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgagccgagctacagtacatgaccctgagactggaaaattgaccacagcacagtacagagtatctaagagtgcctggctctctggctatgaaaatcctgtggtgtctcgaattaatatgagaatacaagatctaacaggactagatgtttccacagcagaggaattacagaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgacaaacaggcttccctttttctcctattgttgtactcttatgtgtctgatatacacatttcctagtcttaactttcaggagtttacaattgactaacactccatgattgattcagtcatgaacctcatcccatgtttcatctgtggacaattgcttactttgtgggttcttttaaaagtaacacgaaatcatcatattgcataaaaccttaaagttctgttggtatcacagaagacaaggcagagtttaaagtgaggaattttatatttaaagaactttttggttggataaaaacataatttgagcatccagttttagtatttcactacatctcagttggtgggtgttaagctagaatgggctgtgtgataggaaacaaatgccttacagatgtgcctaggtgttctgtttacctagtgtcttactctgttttctggatctgaagactagtaataaactaggacactaactgggttccatgtgattgccctttcatatgatcttctaagttgatttttttcctcccaagtcttttttaaagaaagtatactgtattttaccaaccccctctcttttcttttagctcctctgtggtgaattaaacgtacttgagttaaaatatttcgattttttttttttttttaatggaaagtcctgcataacaacactgggccttcttaactaaaatgctcaccacttagcctgtttttttatcccttttttaaaatgacagatgattttgttcaggaattttgctgtttttcttagtgctaataccttgcctcttattcctgctacagcagggtggtaatattggcattctgattaaatactgtgccttaggagactggaagtttaaaaatgtacaagtcctttcagtgatgagggaattgattttttttaaaagtctttttcttagaaagccaaaatgtttgtttttttaagattctgaaatgtgttgtgacaacaatgacctatttatgatcttaaatcttttttaaaaaaaaaaaaaaaa

SEQ ID NO: 11 - protein, 535 aa, Homo sapiens
MKLWVSALLMAWFGVLSCVQAEFFTSIGHMTDLIYAEKELVQSLKEYILVEEAKLSKIKSWANKMEALTSKSAADAEGYLAHPVNAYKLVKRLNTDWPALEDLVLQDSAAGFIANLSVQRQFFPTDEDEIGAAKALMRLQDTYRLDPGTISRGELPGTKYQAMLSVDDCFGMGRSAYNEGDYYHTVLWMEQVLKQLDAGEEATTTKSQVLDYLSYAVFQLGDLHRALELTRRLLSLDPSHERAGGNLRYFEQLLEEEREKTLTNQTEAELATPEGIYERPVDYLPERDVYESLCRGEGVKLTPRRQKRLFCRYHHGNRAPQLLIAPFKEEDEWDSPHIVRYYDVMSDEEIERIKEIAKPKLARATVRDPKTGVLTVASYRVSKSSWLEEDDDPVVARVNRRMQHITGLTVKTAELLQVANYGVGGQYEPHFDFSRNDERDTFKHLGTGNRVATFLNYMSDVEAGGATVFPDLGAAIWPKKGTAVFWYNLLRSGEGDYRTRHAACPVLVGCKWVSNKWFHERGQEFLRPCGSTEVD

SEQ ID NO: 12 - DNA, 2252 bp, Homo sapiens
  source 1..2252: mol_type = genomic DNA
aagggaggaggcgccgagctgaccgggcgacgccgcgggaggttctggaaacgccgggagctgcgagtgtccagctgcggagacccgtgataattcgttaactaattcaacaaacgggacccttctgtgtgccagaaaccgcaagcagttgctaacccagtgggacaggcggattggaagagcgggaaggtcctggcccagagcagtgtgacacttccctctgtgaccatgaaactctgggtgtctgcattgctgatggcctggtttggtgtcctgagctgtgtgcaggccgaattcttcacctctattgggcacatgactgacctgatttatgcagagaaagagctggtgcagtctctgaaagagtacatccttgtggaggaagccaagctttccaagattaagagctgggccaacaaaatggaagccttgactagcaagtcagctgctgatgctgagggctacctggctcaccctgtgaatgcctacaaactggtgaagcggctaaacacagactggcctgcgctggaggaccttgtcctgcaggactcagctgcaggttttatcgccaacctctctgtgcagcggcagttcttccccactgatgaggacgagataggagctgccaaagccctgatgagacttcaggacacatacaggctggacccaggcacaatttccagaggggaacttccaggaaccaagtaccaggcaatgctgagtgtggatgactgctttgggatgggccgctcggcctacaatgaaggggactattatcatacggtgttgtggatggagcaggtgctaaagcagcttgatgccggggaggaggccaccacaaccaagtcacaggtgctggactacctcagctatgctgtcttccagttgggtgatctgcaccgtgccctggagctcacccgccgcctgctctcccttgacccaagccacgaacgagctggagggaatctgcggtactttgagcagttattggaggaagagagagaaaaaacgttaacaaatcagacagaagctgagctagcaaccccagaaggcatctatgagaggcctgtggactacctgcctgagagggatgtttacgagagcctctgtcgtggggagggtgtcaaactgacaccccgtagacagaagaggcttttctgtaggtaccaccatggcaacagggccccacagctgctcattgcccccttcaaagaggaggacgagtgggacagcccgcacatcgtcaggtactacgatgtcatgtctgatgaggaaatcgagaggatcaaggagatcgcaaaacctaaacttgcacgagccaccgttcgtgatcccaagacaggagtcctcactgtcgccagctaccgggtttccaaaagctcctggctagaggaagatgatgaccctgttgtggcccgagtaaatcgtcggatgcagcatatcacagggttaacagtaaagactgcagaattgttacaggttgcaaattatggagtgggaggacagtatgaaccgcacttcgacttctctaggaatgatgagcgagatactttcaagcatttagggacggggaatcgtgtggctactttcttaaactacatgagtgatgtagaagctggtggtgccaccgtcttccctgatctgggggctgcaatttggcctaagaagggtacagctgtgttctggtacaacctcttgcggagcggggaaggtgactaccgaacaagacatgctgcctgccctgtgcttgtgggctgcaagtgggtctccaataagtggttccatgaacgaggacaggagttcttgagaccttgtggatcaacagaagttgactgacatccttttctgtccttccccttcctggtccttcagcccatgtcaacgtgacagacacctttgtatgttcctttgtatgttcctatcaggctgatttttggagaaatgaatgtttgtctggagcagagggagaccatactagggcgactcctgtgtgactgaagtcccagcccttccattcagcctgtgccatccctggccccaaggctaggatcaaagtggctgcagcagagttagctgtctagcgcctagcaaggtgcctttgtacctcaggtgttttaggtgtgagatgtttcagtgaaccaaagttctgataccttgtttacatgtttgtttttatggcatttctatctattgtggctttaccaaaaaataaaatgtccctaccagaagccttaaaaaaaaaaaaaaaaaaa

SEQ ID NO: 13 - protein, 533 aa, Homo sapiens
MKLWVSALLMAWFGVLSCVQAEFFTSIGHMTDLIYAEKELVQSLKEYILVEEAKLSKIKSWANKMEALTSKSAADAEGYLAHPVNAYKLVKRLNTDWPALEDLVLQDSAAGFIANLSVQRQFFPTDEDEIGAAKALMRLQDTYRLDPGTISRGELPGTKYQAMLSVDDCFGMGRSAYNEGDYYHTVLWMEQVLKQLDAGEEATTTKSQVLDYLSYAVFQLGDLHRALELTRRLLSLDPSHERAGGNLRYFEQLLEEEREKTLTNQTEAELATPEGIYERPVDYLPERDVYESLCRGEGVKLTPRRQKRLFCRYHHGNRAPQLLIAPFKEEDEWDSPHIVRYYDVMSDEEIERIKEIAKPKLARATVRDPKTGVLTVASYRVSKSSWLEEDDDPVVARVNRRMQHITGLTVKTAELLQVANYGVGGQYEPHFDFSRRPFDSGLKTEGNRLATFLNYMSDVEAGGATVFPDLGAAIWPKKGTAVFWYNLLRSGEGDYRTRHAACPVLVGCKWVSNKWFHERGQEFLRPCGSTEVD

SEQ ID NO: 14 - DNA, 2246 bp, Homo sapiens
  source 1..2246: mol_type = genomic DNA
aagggaggaggcgccgagctgaccgggcgacgccgcgggaggttctggaaacgccgggagctgcgagtgtccagctgcggagacccgtgataattcgttaactaattcaacaaacgggacccttctgtgtgccagaaaccgcaagcagttgctaacccagtgggacaggcggattggaagagcgggaaggtcctggcccagagcagtgtgacacttccctctgtgaccatgaaactctgggtgtctgcattgctgatggcctggtttggtgtcctgagctgtgtgcaggccgaattcttcacctctattgggcacatgactgacctgatttatgcagagaaagagctggtgcagtctctgaaagagtacatccttgtggaggaagccaagctttccaagattaagagctgggccaacaaaatggaagccttgactagcaagtcagctgctgatgctgagggctacctggctcaccctgtgaatgcctacaaactggtgaagcggctaaacacagactggcctgcgctggaggaccttgtcctgcaggactcagctgcaggttttatcgccaacctctctgtgcagcggcagttcttccccactgatgaggacgagataggagctgccaaagccctgatgagacttcaggacacatacaggctggacccaggcacaatttccagaggggaacttccaggaaccaagtaccaggcaatgctgagtgtggatgactgctttgggatgggccgctcggcctacaatgaaggggactattatcatacggtgttgtggatggagcaggtgctaaagcagcttgatgccggggaggaggccaccacaaccaagtcacaggtgctggactacctcagctatgctgtcttccagttgggtgatctgcaccgtgccctggagctcacccgccgcctgctctcccttgacccaagccacgaacgagctggagggaatctgcggtactttgagcagttattggaggaagagagagaaaaaacgttaacaaatcagacagaagctgagctagcaaccccagaaggcatctatgagaggcctgtggactacctgcctgagagggatgtttacgagagcctctgtcgtggggagggtgtcaaactgacaccccgtagacagaagaggcttttctgtaggtaccaccatggcaacagggccccacagctgctcattgcccccttcaaagaggaggacgagtgggacagcccgcacatcgtcaggtactacgatgtcatgtctgatgaggaaatcgagaggatcaaggagatcgcaaaacctaaacttgcacgagccaccgttcgtgatcccaagacaggagtcctcactgtcgccagctaccgggtttccaaaagctcctggctagaggaagatgatgaccctgttgtggcccgagtaaatcgtcggatgcagcatatcacagggttaacagtaaagactgcagaattgttacaggttgcaaattatggagtgggaggacagtatgaaccgcacttcgacttctctaggcgaccttttgacagcggcctcaaaacagaggggaataggttagcgacgtttcttaactacatgagtgatgtagaagctggtggtgccaccgtcttccctgatctgggggctgcaatttggcctaagaagggtacagctgtgttctggtacaacctcttgcggagcggggaaggtgactaccgaacaagacatgctgcctgccctgtgcttgtgggctgcaagtgggtctccaataagtggttccatgaacgaggacaggagttcttgagaccttgtggatcaacagaagttgactgacatccttttctgtccttccccttcctggtccttcagcccatgtcaacgtgacagacacctttgtatgttcctttgtatgttcctatcaggctgatttttggagaaatgaatgtttgtctggagcagagggagaccatactagggcgactcctgtgtgactgaagtcccagcccttccattcagcctgtgccatccctggccccaaggctaggatcaaagtggctgcagcagagttagctgtctagcgcctagcaaggtgcctttgtacctcaggtgttttaggtgtgagatgtttcagtgaaccaaagttctgataccttgtttacatgtttgtttttatggcatttctatctattgtggctttaccaaaaaataaaatgtccctaccagaagccttaaaaaaaaaaaaaaaaaaa

SEQ ID NO: 15 - protein, 544 aa, Homo sapiens
MGPGARLAALLAVLALGTGDPERAAARGDTFSALTSVARALAPERRLLGLLRRYLRGEEARLRDLTRFYDKVLSLHEDSTTPVANPLLAFTLIKRLQSDWRNVVHSLEASENIRALKDGYEKVEQDLPAFEDLEGAARALMRLQDVYMLNVKGLARGVFQRVTGSAITDLYSPKRLFSLTGDDCFQVGKVAYDMGDYYHAIPWLEEAVSLFRGSYGEWKTEDEASLEDALDHLAFAYFRAGNVSCALSLSREFLLYSPDNKRMARNVLKYERLLAESPNHVVAEAVIQRPNIPHLQTRDTYEGLCQTLGSQPTLYQIPSLYCSYETNSNAYLLLQPIRKEVIHLEPYIALYHDFVSDSEAQKIRELAEPWLQRSVVASGEKQLQVEYRISKSAWLKDTVDPKLVTLNHRIAALTGLDVRPPYAEYLQVVNYGIGGHYEPHFDHATSPSSPLYRMKSGNRVATFMIYLSSVEAGGATAFIYANLSVPVVRNAALFWWNLHRSGEGDSDTLHAGCPVLVGDKWVANKWIHEYGQEFRRPCSSSPED

SEQ ID NO: 16 - DNA, 2299 bp, Homo sapiens
  source 1..2299: mol_type = genomic DNA
gagggcgggagcggctgccgccgcgcggccaggactgtctgaggggaagttcgcgagcgctggctatgggtcctggggcgcggctggcggcgctgctggcggtgctggcgctcgggacaggagacccagaaagggctgcggctcggggcgacacgttctcggcgctgaccagcgtggcgcgcgccctggcgcccgagcgccggctgctggggctgctgaggcggtacctgcgcggggaggaggcgcggctgcgggacctgactagattctacgacaaggtactttctttgcatgaggattcaacaacccctgtggctaaccctctgcttgcatttactctcatcaaacgcctgcagtctgactggaggaatgtggtacatagtctggaggccagtgagaacatccgagctctgaaggatggctatgagaaggtggagcaagaccttccagcctttgaggaccttgagggagcagcaagggccctgatgcggctgcaggacgtgtacatgctcaatgtgaaaggcctggcccgaggtgtctttcagagagtcactggctctgccatcactgacctgtacagccccaaacggctcttttctctcacaggggatgactgcttccaagttggcaaggtggcctatgacatgggggattattaccatgccattccatggctggaggaggctgtcagtctcttccgaggatcttacggagagtggaagacagaggatgaggcaagtctagaagatgccttggatcacttggcctttgcttatttccgggcaggaaatgtttcgtgtgccctcagcctctctcgggagtttcttctctacagcccagataataagaggatggccaggaatgtcttgaaatatgaaaggctcttggcagagagccccaaccacgtggtagctgaggctgtcatccagaggcccaatataccccacctgcagaccagagacacctacgaggggctatgtcagaccctgggttcccagcccactctctaccagatccctagcctctactgttcctatgagaccaattccaacgcctacctgctgctccagcccatccggaaggaggtcatccacctggagccctacattgctctctaccatgacttcgtcagtgactcagaggctcagaaaattagagaacttgcagaaccatggctacagaggtcagtggtggcatcaggggagaagcagttacaagtggagtaccgcatcagcaaaagtgcctggctgaaggacactgttgacccaaaactggtgaccctcaaccaccgcattgctgccctcacaggccttgatgtccggcctccctatgcagagtatctgcaggtggtgaactatggcatcggaggacactatgagcctcactttgaccatgctacgtcaccaagcagccccctctacagaatgaagtcaggaaaccgagttgcaacatttatgatctatctgagctcggtggaagctggaggagccacagccttcatctatgccaacctcagcgtgcctgtggttaggaatgcagcactgttttggtggaacctgcacaggagtggtgaaggggacagtgacacacttcatgctggctgtcctgtcctggtgggagataagtgggtggccaacaagtggatacatgagtatggacaggaattccgcagaccctgcagctccagccctgaagactgaactgttggcagagagaagctggtggagtcctgtggctttccagagaagccaggagccaaaagctggggtaggagaggagaaagcagagcagcctcctggaagaaggccttgtcagctttgtctgtgcctcgcaaatcagaggcaagggagaggttgttaccaggggacactgagaatgtacatttgatctgccccagccacggaagtcagagtaggatgcacagtacaaaggaggggggagtggaggcctgagagggaagtttctggagttcagatactctctgttgggaacaggacatctcaacagtctcaggttcgatcagtgggtcttttggcactttgaaccttgaccacagggaccaagaagtggcaatgaggacacctgcaggaggggctagcctgactcccagaactttaagactttctccccactgccttctgctgcagcccaagcagggagtgtccccctcccagaagcatatcccagatgagtggtacattatataaggattttttttaagttgaaaacaactttcttttctttttgtatgatggttttttaacacagtcattaaaaatgtttataaatcatttttccc

SEQ ID NO: 17 - protein, 604 aa, Homo sapiens
MGPGARLAALLAVLALGTGDPERAAARGDTFSALTSVARALAPERRLLGLLRRYLRGEEARLRDLTRFYDKVLSLHEDSTTPVANPLLAFTLIKRLQSDWRNVVHSLEASENIRALKDGYEKVEQDLPAFEDLEGAARALMRLQDVYMLNVKGLARGVFQRVTGSAITDLYSPKRLFSLTGDDCFQVGKVAYDMGDYYHAIPWLEEAVSLFRGSYGEWKTEDEASLEDALDHLAFAYFRAGNVSCALSLSREFLLYSPDNKRMARNVLKYERLLAESPNHVVAEAVIQRPNIPHLQTRDTYEGLCQTLGSQPTLYQIPSLYCSYETNSNAYLLLQPIRKEVIHLEPYIALYHDFVSDSEAQKIRELAEPWLQRSVVASGEKQLQVEYRISKSAWLKDTVDPKLVTLNHRIAALTGLDVRPPYAEYLQVVNYGIGGHYEPHFDHATSPSSPLYRMKSGNRVATFMIYLSSVEAGGATAFIYANLSVPVVRHCFGGTCTGVVKGTVTHFMLAVLSWWEISGWPTSGYMSMDRNSADPAAPALKTELLAERSWWSPVAFQRSQEPKAGVGEEKAEQPPGRRPCQLCLCLANQRQGRGCYQGTLRMYI

SEQ ID NO: 18 - DNA, 2292 bp, Homo sapiens
  source 1..2292: mol_type = genomic DNA
gagggcgggagcggctgccgccgcgcggccaggactgtctgaggggaagttcgcgagcgctggctatgggtcctggggcgcggctggcggcgctgctggcggtgctggcgctcgggacaggagacccagaaagggctgcggctcggggcgacacgttctcggcgctgaccagcgtggcgcgcgccctggcgcccgagcgccggctgctggggctgctgaggcggtacctgcgcggggaggaggcgcggctgcgggacctgactagattctacgacaaggtactttctttgcatgaggattcaacaacccctgtggctaaccctctgcttgcatttactctcatcaaacgcctgcagtctgactggaggaatgtggtacatagtctggaggccagtgagaacatccgagctctgaaggatggctatgagaaggtggagcaagaccttccagcctttgaggaccttgagggagcagcaagggccctgatgcggctgcaggacgtgtacatgctcaatgtgaaaggcctggcccgaggtgtctttcagagagtcactggctctgccatcactgacctgtacagccccaaacggctcttttctctcacaggggatgactgcttccaagttggcaaggtggcctatgacatgggggattattaccatgccattccatggctggaggaggctgtcagtctcttccgaggatcttacggagagtggaagacagaggatgaggcaagtctagaagatgccttggatcacttggcctttgcttatttccgggcaggaaatgtttcgtgtgccctcagcctctctcgggagtttcttctctacagcccagataataagaggatggccaggaatgtcttgaaatatgaaaggctcttggcagagagccccaaccacgtggtagctgaggctgtcatccagaggcccaatataccccacctgcagaccagagacacctacgaggggctatgtcagaccctgggttcccagcccactctctaccagatccctagcctctactgttcctatgagaccaattccaacgcctacctgctgctccagcccatccggaaggaggtcatccacctggagccctacattgctctctaccatgacttcgtcagtgactcagaggctcagaaaattagagaacttgcagaaccatggctacagaggtcagtggtggcatcaggggagaagcagttacaagtggagtaccgcatcagcaaaagtgcctggctgaaggacactgttgacccaaaactggtgaccctcaaccaccgcattgctgccctcacaggccttgatgtccggcctccctatgcagagtatctgcaggtggtgaactatggcatcggaggacactatgagcctcactttgaccatgctacgtcaccaagcagccccctctacagaatgaagtcaggaaaccgagttgcaacatttatgatctatctgagctcggtggaagctggaggagccacagccttcatctatgccaacctcagcgtgcctgtggttaggcactgttttggtggaacctgcacaggagtggtgaaggggacagtgacacacttcatgctggctgtcctgtcctggtgggagataagtgggtggccaacaagtggatacatgagtatggacaggaattccgcagaccctgcagctccagccctgaagactgaactgttggcagagagaagctggtggagtcctgtggctttccagagaagccaggagccaaaagctggggtaggagaggagaaagcagagcagcctcctggaagaaggccttgtcagctttgtctgtgcctcgcaaatcagaggcaagggagaggttgttaccaggggacactgagaatgtacatttgatctgccccagccacggaagtcagagtaggatgcacagtacaaaggaggggggagtggaggcctgagagggaagtttctggagttcagatactctctgttgggaacaggacatctcaacagtctcaggttcgatcagtgggtcttttggcactttgaaccttgaccacagggaccaagaagtggcaatgaggacacctgcaggaggggctagcctgactcccagaactttaagactttctccccactgccttctgctgcagcccaagcagggagtgtccccctcccagaagcatatcccagatgagtggtacattatataaggattttttttaagttgaaaacaactttcttttctttttgtatgatggttttttaacacagtcattaaaaatgtttataaatcatttttccc

SEQ ID NO: 19 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 20 - DNA, 2596 bp, Homo sapiens
  source 1..2596: mol_type = genomic DNA
gagcctcgaagtccgccggccaatcgaaggcgggccccagcggcgcgtgcgcgccgcggccagcgcgcgcgggcgggggggcaggcgcgccccggacccaggatttataaaggcgaggccgggaccggcgcgcgctctcgtcgcccccgctgtcccggcggcgccaaccgaagcgccccgcctgatccgtgtccgacatgctgcgccgcgctctgctgtgcctggccgtggccgccctggtgcgcgccgacgcccccgaggaggaggaccacgtcctggtgctgcggaaaagcaacttcgcggaggcgctggcggcccacaagtacctgctggtggagttctatgccccttggtgtggccactgcaaggctctggcccctgagtatgccaaagccgctgggaagctgaaggcagaaggttccgagatcaggttggccaaggtggacgccacggaggagtctgacctggcccagcagtacggcgtgcgcggctatcccaccatcaagttcttcaggaatggagacacggcttcccccaaggaatatacagctggcagagaggctgatgacatcgtgaactggctgaagaagcgcacgggcccggctgccaccaccctgcctgacggcgcagctgcagagtccttggtggagtccagcgaggtggctgtcatcggcttcttcaaggacgtggagtcggactctgccaagcagtttttgcaggcagcagaggccatcgatgacataccatttgggatcacttccaacagtgacgtgttctccaaataccagctcgacaaagatggggttgtcctctttaagaagtttgatgaaggccggaacaactttgaaggggaggtcaccaaggagaacctgctggactttatcaaacacaaccagctgccccttgtcatcgagttcaccgagcagacagccccgaagatttttggaggtgaaatcaagactcacatcctgctgttcttgcccaagagtgtgtctgactatgacggcaaactgagcaacttcaaaacagcagccgagagcttcaagggcaagatcctgttcatcttcatcgacagcgaccacaccgacaaccagcgcatcctcgagttctttggcctgaagaaggaagagtgcccggccgtgcgcctcatcaccctggaggaggagatgaccaagtacaagcccgaatcggaggagctgacggcagagaggatcacagagttctgccaccgcttcctggagggcaaaatcaagccccacctgatgagccaggagctgccggaggactgggacaagcagcctgtcaaggtgcttgttgggaagaactttgaagacgtggcttttgatgagaaaaaaaacgtctttgtggagttctatgccccatggtgtggtcactgcaaacagttggctcccatttgggataaactgggagagacgtacaaggaccatgagaacatcgtcatcgccaagatggactcgactgccaacgaggtggaggccgtcaaagtgcacagcttccccacactcaagttctttcctgccagtgccgacaggacggtcattgattacaacggggaacgcacgctggatggttttaagaaattcctggagagcggtggccaggatggggcaggggatgatgacgatctcgaggacctggaagaagcagaggagccagacatggaggaagacgatgatcagaaagctgtgaaagatgaactgtaatacgcaaagccagacccgggcgctgccgagacccctcgggggctgcacacccagcagcagcgcacgcctccgaagcctgcggcctcgcttgaaggagggcgtcgccggaaacccagggaacctctctgaagtgacacctcacccctacacaccgtccgttcacccccgtctcttccttctgcttttcggtttttggaaagggatccatctccaggcagcccaccctggtggggcttgtttcctgaaaccatgatgtactttttcatacatgagtctgtccagagtgcttgctaccgtgttcggagtctcgctgcctccctcccgcgggaggtttctcctctttttgaaaattccgtctgtgggatttttagacatttttcgacatcagggtatttgttccaccttggccaggcctcctcggagaagcttgtcccccgtgtgggagggacggagccggactggacatggtcactcagtaccgcctgcagtgtcgccatgactgatcatggctcttgcatttttgggtaaatggagacttccggatcctgtcagggtgtcccccatgcctggaagaggagctggtggctgccagccctggggcccggcacaggcctgggccttccccttccctcaagccagggctcctcctcctgtcgtgggctcattgtgaccactggcctctctacagcacggcctgtggcctgttcaaggcagaaccacgacccttgactcccgggtggggaggtggccaaggatgctggagctgaatcagacgctgacagttcttcaggcatttctatttcacaatcgaattgaacacattggccaaataaagttgaaattttaccacctgtaaaaaaaaaaaaaaaaaa

SEQ ID NO: 21 - protein, 318 aa, Homo sapiens
MLSESSSFLKGVMLGSIFCALITMLGHIRIGHGNRMHHHEHHHLQAPNKEDILKISEDERMELSKSFRVYCIILVKPKDVSLWAAVKETWTKHCDKAEFFSSENVKVFESINMDTNDMWLMMRKAYKYAFDKYRDQYNWFFLARPTTFAIIENLKYFLLKKDPSQPFYLGHTIKSGDLEYVGMEGGIVLSVESMKRLNSLLNIPEKCPEQGGMIWKISEDKQLAVCLKYAGVFAENAEDADGKDVFNTKSVGLSIKEAMTYHPNQVVEGCCSDMAVTFNGLTPNQMHVMMYGVYRLRAFGHIFNDALVFLPPNGSDND

SEQ ID NO: 22 - DNA, 1743 bp, Homo sapiens
  source 1..1743: mol_type = genomic DNA
cgcgggcgggcaaacggacggctccgcccagccgccggctcctttaccccgccccactacccccgccttcgcggcgctcgtttcctggcgctccccacctttagcgagaccaacgagagaacaccgcctgcagctagaacagcctggtcaggagcgtaacggagtggtgcgccaacgtgagaggaaacccgtgcgcggctgcgctttcctgtccccaagccgttctagacgcgggaaaaatgctttctgaaagcagctcctttttgaagggtgtgatgcttggaagcattttctgtgctttgatcactatgctaggacacattaggattggtcatggaaatagaatgcaccaccatgagcatcatcacctacaagctcctaacaaagaagatatcttgaaaatttcagaggatgagcgcatggagctcagtaagagctttcgagtatactgtattatccttgtaaaacccaaagatgtgagtctttgggctgcagtaaaggagacttggaccaaacactgtgacaaagcagagttcttcagttctgaaaatgttaaagtgtttgagtcaattaatatggacacaaatgacatgtggttaatgatgagaaaagcttacaaatacgcctttgataagtatagagaccaatacaactggttcttccttgcacgccccactacgtttgctatcattgaaaacctaaagtattttttgttaaaaaaggatccatcacagcctttctatctaggccacactataaaatctggagaccttgaatatgtgggtatggaaggaggaattgtcttaagtgtagaatcaatgaaaagacttaacagccttctcaatatcccagaaaagtgtcctgaacagggagggatgatttggaagatatctgaagataaacagctagcagtttgcctgaaatatgctggagtatttgcagaaaatgcagaagatgctgatggaaaagatgtatttaataccaaatctgttgggctttctattaaagaggcaatgacttatcaccccaaccaggtagtagaaggctgttgttcagatatggctgttacttttaatggactgactccaaatcagatgcatgtgatgatgtatggggtataccgccttagggcatttgggcatattttcaatgatgcattggttttcttacctccaaatggttctgacaatgactgagaagtggtagaaaagcgtgaatatgatctttgtataggacgtgtgttgtcattatttgtagtagtaactacatatccaatacagctgtatgtttctttttcttttctaatttggtggcactggtataaccacacattaaagtcagtagtacatttttaaatgagggtggtttttttctttaaaacacatgaacattgtaaatgtgttggaaagaagtgttttaagaataataattttgcaaataaactattaataaatattatatgtgataaattctaaattatgaacattagaaatctgtggggcacatatttttgctgattggttaaaaaattttaacaggtctttagcgttctaagatatgcaaatgatatctctagttgtgaatttgtgattaaagtaaaacttttagctgtgtgttccctttacttctaatactgatttatgttctaagcctccccaagttccaatggatttgccttctcaaaatgtacaactaagcaactaaagaaaattaaagtgaaagttgaaaaataaaaaaaaaaaa

SEQ ID NO: 23 - protein, 418 aa, Homo sapiens
MRSLLLLSAFCLLEAALAAEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL

SEQ ID NO: 24 - DNA, 2289 bp, Homo sapiens
  source 1..2289: mol_type = genomic DNA
agtaggacccaggggccgggaggcgccggcagagggaggggccgggggccggggaggttttgagggaggtctttggctttttttggcggagctggggcgccctccggaagcgtttccaactttccagaagtttctcgggacgggcaggagggggtggggactgccatatatagatcccgggagcaggggagcgggctaagagtagaatcgtgtcgcggctcgagagcgagagtcacgtcccggcgctagcccagcccgacccaggcccaccgtggtgcacgcaaaccacttcctggccatgcgctccctcctgcttctcagcgccttctgcctcctggaggcggccctggccgccgaggtgaagaaacctgcagccgcagcagctcctggcactgcggagaagttgagccccaaggcggccacgcttgccgagcgcagcgccggcctggccttcagcttgtaccaggccatggccaaggaccaggcagtggagaacatcctggtgtcacccgtggtggtggcctcgtcgctagggctcgtgtcgctgggcggcaaggcgaccacggcgtcgcaggccaaggcagtgctgagcgccgagcagctgcgcgacgaggaggtgcacgccggcctgggcgagctgctgcgctcactcagcaactccacggcgcgcaacgtgacctggaagctgggcagccgactgtacggacccagctcagtgagcttcgctgatgacttcgtgcgcagcagcaagcagcactacaactgcgagcactccaagatcaacttccgcgacaagcgcagcgcgctgcagtccatcaacgagtgggccgcgcagaccaccgacggcaagctgcccgaggtcaccaaggacgtggagcgcacggacggcgccctgctagtcaacgccatgttcttcaagccacactgggatgagaaattccaccacaagatggtggacaaccgtggcttcatggtgactcggtcctataccgtgggtgtcatgatgatgcaccggacaggcctctacaactactacgacgacgagaaggaaaagctgcaaatcgtggagatgcccctggcccacaagctctccagcctcatcatcctcatgccccatcacgtggagcctctcgagcgccttgaaaagctgctaaccaaagagcagctgaagatctggatggggaagatgcagaagaaggctgttgccatctccttgcccaagggtgtggtggaggtgacccatgacctgcagaaacacctggctgggctgggcctgactgaggccattgacaagaacaaggccgacttgtcacgcatgtcaggcaagaaggacctgtacctggccagcgtgttccacgccaccgcctttgagttggacacagatggcaacccctttgaccaggacatctacgggcgcgaggagctgcgcagccccaagctgttctacgccgaccaccccttcatcttcctagtgcgggacacccaaagcggctccctgctattcattgggcgcctggtccggcctaagggtgacaagatgcgagacgagttatagggcctcagggtgcacacaggatggcaggaggcatccaaaggctcctgagacacatgggtgctattggggttgggggggaggtgaggtaccagccttggatactccatggggtgggggtggaaaaacagaccggggttcccgtgtgcctgagcggaccttcccagctagaattcactccacttggacatgggccccagataccatgatgctgagcccggaaactccacatcctgtgggacctgggccatagtcattctgcctgccctgaaagtcccagatcaagcctgcctcaatcagtattcatatttatagccaggtaccttctcacctgtgagaccaaattgagctaggggggtcagccagccctcttctgacactaaaacacctcagctgcctccccagctctatcccaacctctcccaactataaaactaggtgctgcagcccctgggaccaggcacccccagaatgacctggccgcagtgaggcggattgagaaggagctcccaggaggggcttctgggcagactctggtcaagaagcatcgtgtctggcgttgtggggatgaactttttgttttgtttcttccttttttagttcttcaaagatagggagggaagggggaacatgagcctttgttgctatcaatccaagaacttatttgtacattttttttttcaataaaacttttccaatgacattttgttggagcgtggaagaaaaaaaaaaaaaaa

SEQ ID NO: 25 - DNA, 8832 bp, synthetic construct
  source 1..8832: mol_type = other DNA; note = Artificial sequence: Plasmid insertion of sequence encoding collagen 7
atgaccctgcggctgctggtggctgccctgtgtgctggcatcctggccgaggctcctagagtgcgggcccagcaccgcgagagagtgacctgcaccagactgtacgccgccgatatcgtgttcctgctggacggctcctcctccatcggccggtccaacttccgggaagtgcggtccttcctggaaggcctggtgctgcctttctccggcgctgcctctgcccagggcgtcagattcgccaccgtgcagtactccgacgacccccggaccgagttcggcctggatgctctgggctctggcggcgacgtgatccgggccatcagagagctgtcctacaagggcggcaacacccggacaggcgccgctatcctgcacgtggccgaccatgtgtttctgccccagctggccagacccggcgtgcccaaagtgtgcatcctgatcaccgacggcaagtcccaggacctggtggacaccgccgctcagagactgaagggccagggcgtgaagctgttcgccgtgggcatcaagaacgccgaccccgaggaactgaagcgggtggcctcccagcctacctccgatttcttcttcttcgtgaacgacttctccatcctgcggaccctgctgcccctggtgtctcggagagtgtgtaccaccgctggcggcgtgccagtgacccggcctcctgacgattctacctccgcccctcgggatctggtgctgtccgagccttccagccagtccctgagagtgcagtggaccgccgcctctggccccgtgaccggctacaaggtgcagtacacccctctgaccggcctgggccagcctctgccttctgagcggcaagaagtgaacgtcccagccggcgagacatccgtgcggctgagaggcctgaggcccctgaccgagtaccaagtgaccgtgatcgccctgtacgccaacagcatcggcgaggccgtgtccggcaccgccagaaccacagccctggaaggacccgagctgaccatccagaacaccaccgcccactccctgctggtcgcttggagatctgtgcctggcgccaccggctatcgcgtgacctggcgagttctgtctggcggccctacccagcagcaagagctgggccctggacagggctctgtgctgctgagggacctggaacccggcaccgactacgaagtgacagtgtccaccctgttcggcagatccgtgggccctgccacctctctgatggccagaaccgacgcctccgtggaacagaccctgaggcctgtgatcctgggccccaccagcatcctgctgagctggaacctggtgcccgaggccagaggctaccggctggaatggcggagagagacaggcctggaacctccccagaaggtggtcctgccctccgacgtgaccagataccagctggatggcctgcagcctggcaccgagtacagactgaccctgtacaccctgctcgagggccacgaggtggccacccctgctacagtggtgcctaccggccctgagctgcccgtgtcccctgtgaccgatctgcaggccaccgagctgcctggccagcgcgtcagagtgtcttggtccccagtgccaggcgctacccagtaccggatcatcgtgcggtccacacagggcgtggaaagaaccctggtgctccccggctcccagaccgccttcgacctggatgatgtgcaggccggcctgagctacaccgtgcgggtgtccgctagagtgggccctagagaaggctccgccagcgtgctgaccgtgcgcagagagcctgaaacccctctggccgtgcccggactgcgggtggtggtgtctgatgccaccagagtcagagtggcctggggccctgtgccaggggcctccggcttcagaatctcctggtccaccggctctggccctgagtcctctcagaccctgccccctgactccaccgccaccgatatcaccggactgcagcccggaaccacctaccaggtggccgtgtctgtgctgaggggcagagaagagggcccagccgccgtgatcgtggccaggacagatcctctgggcccagtgcggaccgtgcacgtgacccaggccagctccagctccgtgaccatcacctggaccagagtccctggcgctacaggctacagagtgtcctggcactctgcccacggccccgagaagtcccagctggtgtctggcgaggccaccgtggctgaactggacggcctcgagcccgacacagagtacacagtgcacgtgcgcgcccatgtggctggcgtggacggacctcctgcttccgtggtcgtgcgcaccgctcctgagcccgtgggaagagtgtcccggctgcagatcctgaacgcctccagcgacgtgctgcggatcacctgggtcggagtgaccggcgctaccgcttacagactggcttggggcagatctgagggcggacccatgcggcatcagatcctgcctggcaacaccgactccgccgagatcagaggactggaaggcggcgtgtcctactctgtgcgcgtgaccgccctcgtgggcgacagagaaggcacccccgtgtccatcgtggtcaccacccctccagaggcccctccagctctgggcaccctgcatgtggtgcagcggggcgagcactccctgagactgagatgggagcctgtgcctcgggcccagggcttcctgctgcattggcagcctgaaggcggccaagagcagtctagggtgctgggccccgagctgtccagctaccacctggacggactggaaccagccacccagtacagagtgcggctgtccgtgctgggacctgccggcgagggaccttctgccgaagtgaccgccaggaccgagtcccctcgggtgccctccatcgagctgagagtggtggataccagcatcgacagcgtgaccctggcctggacccctgtgtcccgggcctcttcctacatcctgtcttggaggcctctgaggggcccaggccaagaggtgccaggctcccctcagacactgccaggcatcagctcctcccagcgcgtgacaggactcgagcctggggtgtcctacatcttctccctgacccccgtcctggacggcgtgcgcggacctgaggcttctgtgacccagaccccagtgtgccccagaggcctggccgacgtggtctttctgcctcacgccacccaggacaacgcccacagagccgaggctaccagacgggtgctcgagagactggtgctggccctgggaccactgggcccacaggctgtgcaagtgggcctgctgtcttactcccaccggccctcccccctgttccccctgaacggctctcacgacctgggcatcatcctgcagcggatccgggacatgccctacatggacccctccggcaacaacctgggcaccgccgtggtcacagcccaccggtacatgctggcccccgatgctcctggcagacggcagcatgtccccggcgtgatggtgctgctcgtggacgagcccctgcggggcgacatcttcagccctatcagagaggcccaggctagcggcctgaacgtggtcatgctgggcatggctggcgccgaccctgagcagctgagaaggctggcccctggcatggactccgtgcagaccttctttgccgtggacgacggccccagcctggatcaggctgtgtctggcctggctaccgccctgtgtcaggcctccttcaccacccagcccagacccgagccttgccccgtgtactgccctaagggccagaagggcgagcccggcgagatgggcctgagaggacaagtgggacctccaggcgatcccggcctgcctggaagaacaggcgctcctggacctcagggccctcctggctctgctaccgctaagggcgagagaggcttcccaggcgccgacggcagacctggctctccaggcagagccggcaatcctggaacacctggcgccccaggcctgaagggatctcctggcttgcctggccctaggggcgaccctggcgaaagaggacctagaggccctaaaggcgagccaggcgcccctggccaagtgatcggcggagaaggacctggactgcccggcagaaagggcgatcctggcccttctggcccacccggcccaagaggtcctctgggagatcctggaccaaggggcccaccaggcctgcccggaacagctatgaagggcgataagggcgacaggggcgagcggggaccaccaggaccaggcgaaggtggaatcgctcccggcgaacctgggctgccaggactgcctggatctcccggaccacagggacctgtgggcccacctggcaagaagggggagaaaggcgactccgaggacggggctccaggattgcccggccaaccaggctctcctggcgaacagggtcccagaggacctcccggcgctatcggcccaaagggggacagaggattccctggcccactgggcgaggctggcgaaaaaggcgaacgcggaccccctggccctgccggcagtagaggacttcctggcgttgccggcagaccaggcgccaagggacctgaaggccctccaggccctaccggaaggcagggcgaaaagggggaacctggcaggccaggcgacccagctgttgtgggaccagccgtggctggacccaaaggcgagaaaggggatgtgggacccgctgggcctagaggcgctactggcgttcagggggaaagaggcccccctggactcgtgctgcctggggatccaggtcctaagggggatcccggcgatagaggcccaatcggcctgaccggcagagctggtccccctggcgattccggtcctcccggggaaaaaggggaccccggtagaccaggtcccccaggccctgttggccctcgcggaagagatggcgaagtgggagaaaagggcgacgaaggacccccaggggacccaggacttccaggcaaggctggggagagaggactgaggggcgctccaggtgtcagaggccctgtcggcgagaagggggatcagggcgatccaggcgaggacggcagaaacggctcccctggctctagtggtccaaaaggcgaccggggagagcctgggcctcctgggccaccaggcagactggtcgataccggacctggggccagagagaagggcgaaccaggggataggggccaagaaggcccacgaggaccaaagggcgacccaggattgcctggcgctcctggcgagaggggcatcgagggctttagaggtccacccggtccccaaggcgaccccggcgttaggggacctgctggggagaagggcgacagaggcccacccggactggacggcagatctggcctggatggcaagcctggcgccgctggcccatctggacctaacggcgctgctggcaaagccggggaccctggacgagatggactgccagggctgcggggagaacagggccttccaggaccttcaggaccacctggcctccctggcaagcccggggaggatggaaagcccggcctgaatggaaaaaacggggaacccggggatcctggggaggacggacgcaagggggaaaagggcgattccggcgcctctggcagagagggcagggacggaccaaaaggggagcgcggagcacccggcattctgggtcctcaggggccacctggattgccaggtccagttggtcctcctggccaggggtttcccggcgtcccaggcggtacagggcctaaaggggatagaggcgagacaggcagcaaaggggaacaggggctcccaggcgaaaggggcttgagaggcgagcctggctccgtgcctaacgtggacagactgctggaaaccgccggcatcaaggcctccgccctgcgcgagatcgtggaaacctgggacgagtcctccggctccttcctgcccgtgcctgagcgcagaaggggcccgaaaggggactctggcgagcaaggaccacccggcaaagagggacccatcggcttccctggggagcgggggttgaaaggcgataggggagatccaggcccacaagggcctccagggctggcacttggagagcgtggtcctccaggaccaagcggactggcaggggagcccggaaagcctggaatccccgggttgcctggtagagccggcggagtgggcgaagcaggcaggcctggggaacgcggagagagaggcgaaaagggcgaaagaggggagcagggccgcgacggtccccccggactacctggaactccagggcccccaggaccccccggacctaaggtgtccgtggatgagcctggccccggactgagcggagaacaaggtccacctggcttgaagggtgccaagggggagccaggctctaacggcgatcaagggcccaagggggatcggggagtgcctggcatcaaaggggaccggggcgaacccggtcctagagggcaagacggaaaccccggcttgccgggcgaacggggaatggctggtcccgagggaaagccaggcttgcagggacctagggggcctcccggtcctgtgggtggacatggcgatccgggtccaccaggtgctccaggactcgctggtccagcaggccctcagggaccatccggcctgaaaggggaaccaggcgaaactggccccccaggcagaggcctgacaggccctactggtgctgtgggcctccctggacctcctggccctagtggactcgtgggccctcagggctctcccggactgccaggccaagtgggcgagactggaaaacccggggctcccggcagggatggcgcttctggaaaagacggcgataggggcagccctggcgtgcccggtagtccagggctacctggccctgtgggtcccaaaggggagcctggacctacaggcgcaccaggccaggctgtagtggggctgcctggcgctaaaggcgagaagggtgctcctggcggcctggctggcgatctcgttggagaacctggcgccaagggcgaccgtggcttgccaggacctcgcggcgagaaaggcgaagctggcagagctggcgagcctggggacccaggcgaagatggccagaaaggcgctcccggccctaagggattcaagggcgatccgggcgtgggcgtgccaggctctccaggtcctcctggaccacccggtgtcaagggcgatttgggccttcctggcctgccaggggcacctggcgtcgtgggctttcctggacagaccggcccacggggagagatgggacagccaggccccagcggagaaagagggctggctggcccgcctggcagggaaggcataccaggcccattggggcctccaggcccacctggatctgtggggcctcctggcgcctctggactgaaaggcgacaaaggcgatcctggtgtcggcctgccaggcccaagaggcgagaggggagagcccggcatcaggggcgaagatggacggcctggccaagagggccctcggggattgaccggccctcctggatccagaggcgaacggggggagaagggggacgtgggctctgctggcctcaaaggcgacaagggggactccgccgtgattctgggccctcccggacctcggggagctaagggggacatgggagagaggggtccacggggactggatggggacaagggaccacgcggagacaacggcgacccgggggataagggctccaagggcgaacctggcgataagggatccgctggactgcctggcctgaggggcctgctgggacctcaaggacaaccaggcgccgcaggcatccctggcgaccctggatctcctggaaaggacggcgtgcccggcatccgcggagaaaagggggatgtcggcttcatgggccccagggggctgaagggggaaaggggagtgaagggcgcttgcggcctcgatggggaaaagggggacaagggggaggctggccctccaggacgacctggactggctggccacaagggcgaaatgggagagccaggcgtgcccggacagtccggcgctccaggcaaagagggcctgatcggccccaaaggcgatagaggatttgacggccagcctggcccaaagggcgatcaaggcgaaaaaggggagagaggcacccccggcatcggcggctttccaggcccctctggaaacgatggctctgccggcccacctgggccacctggtagtgtgggaccaagaggccccgagggactgcagggacagaaaggcgagagagggccccctggcgagagagttgtgggagcacctggcgttcccggcgcacccggcgaaaggggagaacaaggcagacctggaccagccggaccccgtggggaaaaaggcgaggccgccctgaccgaggacgacatcagaggcttcgtgcggcaagagatgtcccagcactgcgcctgtcagggccagtttatcgcctccggcagcagacccctgccttcctacgctgccgataccgccggctctcagctgcacgctgtgcctgtgctccgggtgtcccacgccgaggaagaggaaagagtccctcctgaggacgacgagtacagcgagtactctgagtattccgtggaagagtaccaggatcccgaggccccttgggacagcgacgacccttgctccctgcctctggatgagggctcctgcaccgcctacaccctgagatggtatcaccgggccgtgacaggctccaccgaggcctgtcaccctttcgtgtatggcggctgcggcggcaacgccaatagattcggcacccgcgaggcctgcgagcggagatgtcctcccagagtggtgcagtcccagggcaccggcacagcccaggac

SEQ ID NO: 26 - DNA, 17984 bp, synthetic construct
  source 1..17984: mol_type = other DNA; note = Artificial sequence: Vector for collagen 7
tcgataggtaccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcacaagcttcgaagtttaaactgagtgccgccaccatgaccctgcggctgctggtggctgccctgtgtgctggcatcctggccgaggctcctagagtgcgggcccagcaccgcgagagagtgacctgcaccagactgtacgccgccgatatcgtgttcctgctggacggctcctcctccatcggccggtccaacttccgggaagtgcggtccttcctggaaggcctggtgctgcctttctccggcgctgcctctgcccagggcgtcagattcgccaccgtgcagtactccgacgacccccggaccgagttcggcctggatgctctgggctctggcggcgacgtgatccgggccatcagagagctgtcctacaagggcggcaacacccggacaggcgccgctatcctgcacgtggccgaccatgtgtttctgccccagctggccagacccggcgtgcccaaagtgtgcatcctgatcaccgacggcaagtcccaggacctggtggacaccgccgctcagagactgaagggccagggcgtgaagctgttcgccgtgggcatcaagaacgccgaccccgaggaactgaagcgggtggcctcccagcctacctccgatttcttcttcttcgtgaacgacttctccatcctgcggaccctgctgcccctggtgtctcggagagtgtgtaccaccgctggcggcgtgccagtgacccggcctcctgacgattctacctccgcccctcgggatctggtgctgtccgagccttccagccagtccctgagagtgcagtggaccgccgcctctggccccgtgaccggctacaaggtgcagtacacccctctgaccggcctgggccagcctctgccttctgagcggcaagaagtgaacgtcccagccggcgagacatccgtgcggctgagaggcctgaggcccctgaccgagtaccaagtgaccgtgatcgccctgtacgccaacagcatcggcgaggccgtgtccggcaccgccagaaccacagccctggaaggacccgagctgaccatccagaacaccaccgcccactccctgctggtcgcttggagatctgtgcctggcgccaccggctatcgcgtgacctggcgagttctgtctggcggccctacccagcagcaagagctgggccctggacagggctctgtgctgctgagggacctggaacccggcaccgactacgaagtgacagtgtccaccctgttcggcagatccgtgggccctgccacctctctgatggccagaaccgacgcctccgtggaacagaccctgaggcctgtgatcctgggccccaccagcatcctgctgagctggaacctggtgcccgaggccagaggctaccggctggaatggcggagagagacaggcctggaacctccccagaaggtggtcctgccctccgacgtgaccagataccagctggatggcctgcagcctggcaccgagtacagactgaccctgtacaccctgctcgagggccacgaggtggccacccctgctacagtggtgcctaccggccctgagctgcccgtgtcccctgtgaccgatctgcaggccaccgagctgcctggccagcgcgtcagagtgtcttggtccccagtgccaggcgctacccagtaccggatcatcgtgcggtccacacagggcgtggaaagaaccctggtgctccccggctcccagaccgccttcgacctggatgatgtgcaggccggcctgagctacaccgtgcgggtgtccgctagagtgggccctagagaaggctccgccagcgtgctgaccgtgcgcagagagcctgaaacccctctggccgtgcccggactgcgggtggtggtgtctgatgccaccagagtcagagtggcctggggccctgtgccaggggcctccggcttcagaatctcctggtccaccggctctggccctgagtcctctcagaccctgccccctgactccaccgccaccgatatcaccggactgcagcccggaaccacctaccaggtggccgtgtctgtgctgaggggcagagaagagggcccagccgccgtgatcgtggccaggacagatcctctgggcccagtgcggaccgtgcacgtgacccaggccagctccagctccgtgaccatcacctggaccagagtccctggcgctacaggctacagagtgtcctggcactctgcccacggccccgagaagtcccagctggtgtctggcgaggccaccgtggctgaactggacggcctcgagcccgacacagagtacacagtgcacgtgcgcgcccatgtggctggcgtggacggacctcctgcttccgtggtcgtgcgcaccgctcctgagcccgtgggaagagtgtcccggctgcagatcctgaacgcctccagcgacgtgctgcggatcacctgggtcggagtgaccggcgctaccgcttacagactggcttggggcagatctgagggcggacccatgcggcatcagatcctgcctggcaacaccgactccgccgagatcagaggactggaaggcggcgtgtcctactctgtgcgcgtgaccgccctcgtgggcgacagagaaggcacccccgtgtccatcgtggtcaccacccctccagaggcccctccagctctgggcaccctgcatgtggtgcagcggggcgagcactccctgagactgagatgggagcctgtgcctcgggcccagggcttcctgctgcattggcagcctgaaggcggccaagagcagtctagggtgctgggccccgagctgtccagctaccacctggacggactggaaccagccacccagtacagagtgcggctgtccgtgctgggacctgccggcgagggaccttctgccgaagtgaccgccaggaccgagtcccctcgggtgccctccatcgagctgagagtggtggataccagcatcgacagcgtgaccctggcctggacccctgtgtcccgggcctcttcctacatcctgtcttggaggcctctgaggggcccaggccaagaggtgccaggctcccctcagacactgccaggcatcagctcctcccagcgcgtgacaggactcgagcctggggtgtcctacatcttctccctgacccccgtcctggacggcgtgcgcggacctgaggcttctgtgacccagaccccagtgtgccccagaggcctggccgacgtggtctttctgcctcacgccacccaggacaacgcccacagagccgaggctaccagacgggtgctcgagagactggtgctggccctgggaccactgggcccacaggctgtgcaagtgggcctgctgtcttactcccaccggccctcccccctgttccccctgaacggctctcacgacctgggcatcatcctgcagcggatccgggacatgccctacatggacccctccggcaacaacctgggcaccgccgtggtcacagcccaccggtacatgctggcccccgatgctcctggcagacggcagcatgtccccggcgtgatggtgctgctcgtggacgagcccctgcggggcgacatcttcagccctatcagagaggcccaggctagcggcctgaacgtggtcatgctgggcatggctggcgccgaccctgagcagctgagaaggctggcccctggcatggactccgtgcagaccttctttgccgtggacgacggccccagcctggatcaggctgtgtctggcctggctaccgccctgtgtcaggcctccttcaccacccagcccagacccgagccttgccccgtgtactgccctaagggccagaagggcgagcccggcgagatgggcctgagaggacaagtgggacctccaggcgatcccggcctgcctggaagaacaggcgctcctggacctcagggccctcctggctctgctaccgctaagggcgagagaggcttcccaggcgccgacggcagacctggctctccaggcagagccggcaatcctggaacacctggcgccccaggcctgaagggatctcctggcttgcctggccctaggggcgaccctggcgaaagaggacctagaggccctaaaggcgagccaggcgcccctggccaagtgatcggcggagaaggacctggactgcccggcagaaagggcgatcctggcccttctggcccacccggcccaagaggtcctctgggagatcctggaccaaggggcccaccaggcctgcccggaacagctatgaagggcgataagggcgacaggggcgagcggggaccaccaggaccaggcgaaggtggaatcgctcccggcgaacctgggctgccaggactgcctggatctcccggaccacagggacctgtgggcccacctggcaagaagggggagaaaggcgactccgaggacggggctccaggattgcccggccaaccaggctctcctggcgaacagggtcccagaggacctcccggcgctatcggcccaaagggggacagaggattccctggcccactgggcgaggctggcgaaaaaggcgaacgcggaccccctggccctgccggcagtagaggacttcctggcgttgccggcagaccaggcgccaagggacctgaaggccctccaggccctaccggaaggcagggcgaaaagggggaacctggcaggccaggcgacccagctgttgtgggaccagccgtggctggacccaaaggcgagaaaggggatgtgggacccgctgggcctagaggcgctactggcgttcagggggaaagaggcccccctggactcgtgctgcctggggatccaggtcctaagggggatcccggcgatagaggcccaatcggcctgaccggcagagctggtccccctggcgattccggtcctcccggggaaaaaggggaccccggtagaccaggtcccccaggccctgttggccctcgcggaagagatggcgaagtgggagaaaagggcgacgaaggacccccaggggacccaggacttccaggcaaggctggggagagaggactgaggggcgctccaggtgtcagaggccctgtcggcgagaagggggatcagggcgatccaggcgaggacggcagaaacggctcccctggctctagtggtccaaaaggcgaccggggagagcctgggcctcctgggccaccaggcagactggtcgataccggacctggggccagagagaagggcgaaccaggggataggggccaagaaggcccacgaggaccaaagggcgacccaggattgcctggcgctcctggcgagaggggcatcgagggctttagaggtccacccggtccccaaggcgaccccggcgttaggggacctgctggggagaagggcgacagaggcccacccggactggacggcagatctggcctggatggcaagcctggcgccgctggcccatctggacctaacggcgctgctggcaaagccggggaccctggacgagatggactgccagggctgcggggagaacagggccttccaggaccttcaggaccacctggcctccctggcaagcccggggaggatggaaagcccggcctgaatggaaaaaacggggaacccggggatcctggggaggacggacgcaagggggaaaagggcgattccggcgcctctggcagagagggcagggacggaccaaaaggggagcgcggagcacccggcattctgggtcctcaggggccacctggattgccaggtccagttggtcctcctggccaggggtttcccggcgtcccaggcggtacagggcctaaaggggatagaggcgagacaggcagcaaaggggaacaggggctcccaggcgaaaggggcttgagaggcgagcctggctccgtgcctaacgtggacagactgctggaaaccgccggcatcaaggcctccgccctgcgcgagatcgtggaaacctgggacgagtcctccggctccttcctgcccgtgcctgagcgcagaaggggcccgaaaggggactctggcgagcaaggaccacccggcaaagagggacccatcggcttccctggggagcgggggttgaaaggcgataggggagatccaggcccacaagggcctccagggctggcacttggagagcgtggtcctccaggaccaagcggactggcaggggagcccggaaagcctggaatccccgggttgcctggtagagccggcggagtgggcgaagcaggcaggcctggggaacgcggagagagaggcgaaaagggcgaaagaggggagcagggccgcgacggtccccccggactacctggaactccagggcccccaggaccccccggacctaaggtgtccgtggatgagcctggccccggactgagcggagaacaaggtccacctggcttgaagggtgccaagggggagccaggctctaacggcgatcaagggcccaagggggatcggggagtgcctggcatcaaaggggaccggggcgaacccggtcctagagggcaagacggaaaccccggcttgccgggcgaacggggaatggctggtcccgagggaaagccaggcttgcagggacctagggggcctcccggtcctgtgggtggacatggcgatccgggtccaccaggtgctccaggactcgctggtccagcaggccctcagggaccatccggcctgaaaggggaaccaggcgaaactggccccccaggcagaggcctgacaggccctactggtgctgtgggcctccctggacctcctggccctagtggactcgtgggccctcagggctctcccggactgccaggccaagtgggcgagactggaaaacccggggctcccggcagggatggcgcttctggaaaagacggcgataggggcagccctggcgtgcccggtagtccagggctacctggccctgtgggtcccaaaggggagcctggacctacaggcgcaccaggccaggctgtagtggggctgcctggcgctaaaggcgagaagggtgctcctggcggcctggctggcgatctcgttggagaacctggcgccaagggcgaccgtggcttgccaggacctcgcggcgagaaaggcgaagctggcagagctggcgagcctggggacccaggcgaagatggccagaaaggcgctcccggccctaagggattcaagggcgatccgggcgtgggcgtgccaggctctccaggtcctcctggaccacccggtgtcaagggcgatttgggccttcctggcctgccaggggcacctggcgtcgtgggctttcctggacagaccggcccacggggagagatgggacagccaggccccagcggagaaagagggctggctggcccgcctggcagggaaggcataccaggcccattggggcctccaggcccacctggatctgtggggcctcctggcgcctctggactgaaaggcgacaaaggcgatcctggtgtcggcctgccaggcccaagaggcgagaggggagagcccggcatcaggggcgaagatggacggcctggccaagagggccctcggggattgaccggccctcctggatccagaggcgaacggggggagaagggggacgtgggctctgctggcctcaaaggcgacaagggggactccgccgtgattctgggccctcccggacctcggggagctaagggggacatgggagagaggggtccacggggactggatggggacaagggaccacgcggagacaacggcgacccgggggataagggctccaagggcgaacctggcgataagggatccgctggactgcctggcctgaggggcctgctgggacctcaaggacaaccaggcgccgcaggcatccctggcgaccctggatctcctggaaaggacggcgtgcccggcatccgcggagaaaagggggatgtcggcttcatgggccccagggggctgaagggggaaaggggagtgaagggcgcttgcggcctcgatggggaaaagggggacaagggggaggctggccctccaggacgacctggactggctggccacaagggcgaaatgggagagccaggcgtgcccggacagtccggcgctccaggcaaagagggcctgatcggccccaaaggcgatagaggatttgacggccagcctggcccaaagggcgatcaaggcgaaaaaggggagagaggcacccccggcatcggcggctttccaggcccctctggaaacgatggctctgccggcccacctgggccacctggtagtgtgggaccaagaggccccgagggactgcagggacagaaaggcgagagagggccccctggcgagagagttgtgggagcacctggcgttcccggcgcacccggcgaaaggggagaacaaggcagacctggaccagccggaccccgtggggaaaaaggcgaggccgccctgaccgaggacgacatcagaggcttcgtgcggcaagagatgtcccagcactgcgcctgtcagggccagtttatcgcctccggcagcagacccctgccttcctacgctgccgataccgccggctctcagctgcacgctgtgcctgtgctccgggtgtcccacgccgaggaagaggaaagagtccctcctgaggacgacgagtacagcgagtactctgagtattccgtggaagagtaccaggatcccgaggccccttgggacagcgacgacccttgctccctgcctctggatgagggctcctgcaccgcctacaccctgagatggtatcaccgggccgtgacaggctccaccgaggcctgtcaccctttcgtgtatggcggctgcggcggcaacgccaatagattcggcacccgcgaggcctgcgagcggagatgtcctcccagagtggtgcagtcccagggcaccggcacagcccaggactgatagtctagagtcggggcggccggccgctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatccgagtgtcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccctttataaaaccacaatataatggagtgctataatttcaaacagtgtttggtctgctggcagagtggtcattctaacagcagtcacagtagagtagaaataagactgcagtatatctaaggcaaaaagctgaggtttcaggagcttgaaggtaaagaggaagaaagaaatgggaatgggaattggaaagacaaatatcgttaagagaaaattgcttttaggagaggggaaagaatctatgtgtacttaagactatggaatcaatcccatttaagctgggaaactagtttcatatataactaataaattttatttacagaatatctatttacctgatctaggcttcaagccaaagggactgtgtgaaaaaccatcagttctgtcatattcctaaaaaaaaattaaaaagttaaaaataaataaataataaaacttcttttctttcaaaataatcaaggtgcttattcacatccattccaatttggggaaatacttattttcctatgattagcgaagagaaaagtaacttgcatttcaattcaagttgatacatgtcacttttaagaggtcaactaatatttgctagttgagctaaccatataggctttaaatactttcatagtagaaagaaaatgaaaatcattagtgaactgtataaaatagatcatactttttgaaagaatcagactgaagtttccgaaaaaaagaagtaagctagcttcaatgaaaaggtaagtgaatttagcatttactcagcatctactatggacttaacacctaacagtagataatctgaaggcaaacatatttgtatagggactgcagaatgatagatgataaatatcatctcttctatttgaatgaatattttttcaaatctttcacacacagtggtttgctatggaaagatttgtagtacattaaacaaatctgaagatggagttagaaagctagcttaggctatgttttgagcacaacatataatttctctgtgattgtttcttcatctttcaaatgaggttactgtgaagattaaatgagataactaaatgatgataaaataatgtaatcttagcagcaccttatttaatctgtgcaacaactctgtgaagtgagtagggctcagcttcagtcacttctctgccatttattaactaagatagtttggaaagttacccatctcttcagctgtaaaatgatgaggatcatacctattttatggggctgcttttaggtacaaatatacaggcaagcactttgttaatactaaagcattacaccaattagttttactcttttccattcacacatgaaattaatgtaatcagaattctgtagattacctaaatcttctgttaacacgtgatatgcagttcaggttaaatgtcagttgagttaccaaagcacatacatactcaccaccctatccaaatctacaagcctcccagtttgtcttcactattttggttaaattaatatgaattcctagatgaaaatttcactgatccaaatgaaataaaaaatatattacaaaactcacacctgtaatctcaacattttgggaggccaaggcaggtagatcacttgaggccaggagttcaagaccagcctgatcaacatggtgaaaccctgtctctactaaaaatacaaaaattagccaggtgtggtggcatgtgcctgtagtcctacctactcgggaggctgaggcacaagaatcgcttgaatgtgggaggtggaggttgcagtgacctgagatcgtgccactgcactccagcctaggcaacagagtgagatcatgtgtcatatatatatatatatatatatatatatatatatatatatacacacacacacacatatatatatacacatatatatacgtatatatatatatgtatatatatacatatatatacatatatatatatacgtatatatatacgtatatatatatcaatgtaaattatttgggaaatttggtatgaatagtcttccctgtgaacacagatcataaaatcatatatcaagcagacaaataagtagtagtcacttatatgcttatacttgtaacttaaagtaaaagaattacaaaagcatatgacaaagactaattttaagatatcctaatttaaattgttttctaaaagtgtgtataccattttacctatcatatgaataatttagaaacatgtttataaaattaatgtccaaatccattcaaaagttttgtaatgcagatcacccacaacaacaaagaatcctagcctattaaaaaagcaacaccacctacatataatgaaatattagcagcatctatgtaaccaaagttacacagtgaatttgggccatccaacactttgagcaaagtgttgaattcatcaaatgaatgtgtaatcatttacttactaatgccaatacactttaaggtaatcttaagtagaagagatagagtttagaattttttaaatttatctcttgttgtaaagcaatagacttgaataaataaattagaagaatcagtcattcaagccaccagagtatttgatcgagatttcacaaactctaactttctgatacccattctcccaaaaacgtgtaacctcctgtcgataggaacaacccactgcagggatgtttctcgtggaaaaaggaaatttcttttgcattggtttcagacctaactggttacaagaaaaaccaaaggccattgcacaatgctgaagtacttttttcaaatttaaaatttgaaagttgttcttaaaatctatcatttattttaaaatacggatgaatgagaaagcatagatttgataaagtgaattcttttctgcaatctacagacacttccaaaaatcactacagacactacagacactacagaaaatcataaataaacaagtgctagtatcaatatttttaccaaaaaatggcattcttagaattttttataggctagaaggtttgtacaaactaatctgccacggattttaaaatatgagtgaataaattatattgcaaaaaaaatcaggttacagagaactggcaaggaagactcttatgtaaaacacagaaaacatacaaaacgtatttttaagacaaataaaaacagaacttgtacctcagatgatactggagattgtgttgacatattagcattatcactgtcttgctaaaacataaaaataaaaagatggaagatgaaattacaatacaaatgatgatttaaacatataaaaggaaaataaaaattgttctgaccaactactaaaggaagacctactaaagatatgccatccagcacattgccactctacatgtggtctgtaaaccagcagcatagggatccgtcgatcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgatcataattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagctgcggcccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctcta

SEQ ID NO: 27 - DNA, 18422 bp, synthetic construct
  source 1..18422: mol_type = other DNA; note = Artificial sequence: Vector for collagen 7
tcgataggtaccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcacaagcttcgaagtttaaactgagtgccgccaccatgaccctgcggctgctggtggctgccctgtgtgctggcatcctggccgaggctcctagagtgcgggcccagcaccgcgagagagtgacctgcaccagactgtacgccgccgatatcgtgttcctgctggacggctcctcctccatcggccggtccaacttccgggaagtgcggtccttcctggaaggcctggtgctgcctttctccggcgctgcctctgcccagggcgtcagattcgccaccgtgcagtactccgacgacccccggaccgagttcggcctggatgctctgggctctggcggcgacgtgatccgggccatcagagagctgtcctacaagggcggcaacacccggacaggcgccgctatcctgcacgtggccgaccatgtgtttctgccccagctggccagacccggcgtgcccaaagtgtgcatcctgatcaccgacggcaagtcccaggacctggtggacaccgccgctcagagactgaagggccagggcgtgaagctgttcgccgtgggcatcaagaacgccgaccccgaggaactgaagcgggtggcctcccagcctacctccgatttcttcttcttcgtgaacgacttctccatcctgcggaccctgctgcccctggtgtctcggagagtgtgtaccaccgctggcggcgtgccagtgacccggcctcctgacgattctacctccgcccctcgggatctggtgctgtccgagccttccagccagtccctgagagtgcagtggaccgccgcctctggccccgtgaccggctacaaggtgcagtacacccctctgaccggcctgggccagcctctgccttctgagcggcaagaagtgaacgtcccagccggcgagacatccgtgcggctgagaggcctgaggcccctgaccgagtaccaagtgaccgtgatcgccctgtacgccaacagcatcggcgaggccgtgtccggcaccgccagaaccacagccctggaaggacccgagctgaccatccagaacaccaccgcccactccctgctggtcgcttggagatctgtgcctggcgccaccggctatcgcgtgacctggcgagttctgtctggcggccctacccagcagcaagagctgggccctggacagggctctgtgctgctgagggacctggaacccggcaccgactacgaagtgacagtgtccaccctgttcggcagatccgtgggccctgccacctctctgatggccagaaccgacgcctccgtggaacagaccctgaggcctgtgatcctgggccccaccagcatcctgctgagctggaacctggtgcccgaggccagaggctaccggctggaatggcggagagagacaggcctggaacctccccagaaggtggtcctgccctccgacgtgaccagataccagctggatggcctgcagcctggcaccgagtacagactgaccctgtacaccctgctcgagggccacgaggtggccacccctgctacagtggtgcctaccggccctgagctgcccgtgtcccctgtgaccgatctgcaggccaccgagctgcctggccagcgcgtcagagtgtcttggtccccagtgccaggcgctacccagtaccggatcatcgtgcggtccacacagggcgtggaaagaaccctggtgctccccggctcccagaccgccttcgacctggatgatgtgcaggccggcctgagctacaccgtgcgggtgtccgctagagtgggccctagagaaggctccgccagcgtgctgaccgtgcgcagagagcctgaaacccctctggccgtgcccggactgcgggtggtggtgtctgatgccaccagagtcagagtggcctggggccctgtgccaggggcctccggcttcagaatctcctggtccaccggctctggccctgagtcctctcagaccctgccccctgactccaccgccaccgatatcaccggactgcagcccggaaccacctaccaggtggccgtgtctgtgctgaggggcagagaagagggcccagccgccgtgatcgtggccaggacagatcctctgggcccagtgcggaccgtgcacgtgacccaggccagctccagctccgtgaccatcacctggaccagagtccctggcgctacaggctacagagtgtcctggcactctgcccacggccccgagaagtcccagctggtgtctggcgaggccaccgtggctgaactggacggcctcgagcccgacacagagtacacagtgcacgtgcgcgcccatgtggctggcgtggacggacctcctgcttccgtggtcgtgcgcaccgctcctgagcccgtgggaagagtgtcccggctgcagatcctgaacgcctccagcgacgtgctgcggatcacctgggtcggagtgaccggcgctaccgcttacagactggcttggggcagatctgagggcggacccatgcggcatcagatcctgcctggcaacaccgactccgccgagatcagaggactggaaggcggcgtgtcctactctgtgcgcgtgaccgccctcgtgggcgacagagaaggcacccccgtgtccatcgtggtcaccacccctccagaggcccctccagctctgggcaccctgcatgtggtgcagcggggcgagcactccctgagactgagatgggagcctgtgcctcgggcccagggcttcctgctgcattggcagcctgaaggcggccaagagcagtctagggtgctgggccccgagctgtccagctaccacctggacggactggaaccagccacccagtacagagtgcggctgtccgtgctgggacctgccggcgagggaccttctgccgaagtgaccgccaggaccgagtcccctcgggtgccctccatcgagctgagagtggtggataccagcatcgacagcgtgaccctggcctggacccctgtgtcccgggcctcttcctacatcctgtcttggaggcctctgaggggcccaggccaagaggtgccaggctcccctcagacactgccaggcatcagctcctcccagcgcgtgacaggactcgagcctggggtgtcctacatcttctccctgacccccgtcctggacggcgtgcgcggacctgaggcttctgtgacccagaccccagtgtgccccagaggcctggccgacgtggtctttctgcctcacgccacccaggacaacgcccacagagccgaggctaccagacgggtgctcgagagactggtgctggccctgggaccactgggcccacaggctgtgcaagtgggcctgctgtcttactcccaccggccctcccccctgttccccctgaacggctctcacgacctgggcatcatcctgcagcggatccgggacatgccctacatggacccctccggcaacaacctgggcaccgccgtggtcacagcccaccggtacatgctggcccccgatgctcctggcagacggcagcatgtccccggcgtgatggtgctgctcgtggacgagcccctgcggggcgacatcttcagccctatcagagaggcccaggctagcggcctgaacgtggtcatgctgggcatggctggcgccgaccctgagcagctgagaaggctggcccctggcatggactccgtgcagaccttctttgccgtggacgacggccccagcctggatcaggctgtgtctggcctggctaccgccctgtgtcaggcctccttcaccacccagcccagacccgagccttgccccgtgtactgccctaagggccagaagggcgagcccggcgagatgggcctgagaggacaagtgggacctccaggcgatcccggcctgcctggaagaacaggcgctcctggacctcagggccctcctggctctgctaccgctaagggcgagagaggcttcccaggcgccgacggcagacctggctctccaggcagagccggcaatcctggaacacctggcgccccaggcctgaagggatctcctggcttgcctggccctaggggcgaccctggcgaaagaggacctagaggccctaaaggcgagccaggcgcccctggccaagtgatcggcggagaaggacctggactgcccggcagaaagggcgatcctggcccttctggcccacccggcccaagaggtcctctgggagatcctggaccaaggggcccaccaggcctgcccggaacagctatgaagggcgataagggcgacaggggcgagcggggaccaccaggaccaggcgaaggtggaatcgctcccggcgaacctgggctgccaggactgcctggatctcccggaccacagggacctgtgggcccacctggcaagaagggggagaaaggcgactccgaggacggggctccaggattgcccggccaaccaggctctcctggcgaacagggtcccagaggacctcccggcgctatcggcccaaagggggacagaggattccctggcccactgggcgaggctggcgaaaaaggcgaacgcggaccccctggccctgccggcagtagaggacttcctggcgttgccggcagaccaggcgccaagggacctgaaggccctccaggccctaccggaaggcagggcgaaaagggggaacctggcaggccaggcgacccagctgttgtgggaccagccgtggctggacccaaaggcgagaaaggggatgtgggacccgctgggcctagaggcgctactggcgttcagggggaaagaggcccccctggactcgtgctgcctggggatccaggtcctaagggggatcccggcgatagaggcccaatcggcctgaccggcagagctggtccccctggcgattccggtcctcccggggaaaaaggggaccccggtagaccaggtcccccaggccctgttggccctcgcggaagagatggcgaagtgggagaaaagggcgacgaaggacccccaggggacccaggacttccaggcaaggctggggagagaggactgaggggcgctccaggtgtcagaggccctgtcggcgagaagggggatcagggcgatccaggcgaggacggcagaaacggctcccctggctctagtggtccaaaaggcgaccggggagagcctgggcctcctgggccaccaggcagactggtcgataccggacctggggccagagagaagggcgaaccaggggataggggccaagaaggcccacgaggaccaaagggcgacccaggattgcctggcgctcctggcgagaggggcatcgagggctttagaggtccacccggtccccaaggcgaccccggcgttaggggacctgctggggagaagggcgacagaggcccacccggactggacggcagatctggcctggatggcaagcctggcgccgctggcccatctggacctaacggcgctgctggcaaagccggggaccctggacgagatggactgccagggctgcggggagaacagggccttccaggaccttcaggaccacctggcctccctggcaagcccggggaggatggaaagcccggcctgaatggaaaaaacggggaacccggggatcctggggaggacggacgcaagggggaaaagggcgattccggcgcctctggcagagagggcagggacggaccaaaaggggagcgcggagcacccggcattctgggtcctcaggggccacctggattgccaggtccagttggtcctcctggccaggggtttcccggcgtcccaggcggtacagggcctaaaggggatagaggcgagacaggcagcaaaggggaacaggggctcccaggcgaaaggggcttgagaggcgagcctggctccgtgcctaacgtggacagactgctggaaaccgccggcatcaaggcctccgccctgcgcgagatcgtggaaacctgggacgagtcctccggctccttcctgcccgtgcctgagcgcagaaggggcccgaaaggggactctggcgagcaaggaccacccggcaaagagggacccatcggcttccctggggagcgggggttgaaaggcgataggggagatccaggcccacaagggcctccagggctggcacttggagagcgtggtcctccaggaccaagcggactggcaggggagcccggaaagcctggaatccccgggttgcctggtagagccggcggagtgggcgaagcaggcaggcctggggaacgcggagagagaggcgaaaagggcgaaagaggggagcagggccgcgacggtccccccggactacctggaactccagggcccccaggaccccccggacctaaggtgtccgtggatgagcctggccccggactgagcggagaacaaggtccacctggcttgaagggtgccaagggggagccaggctctaacggcgatcaagggcccaagggggatcggggagtgcctggcatcaaaggggaccggggcgaacccggtcctagagggcaagacggaaaccccggcttgccgggcgaacggggaatggctggtcccgagggaaagccaggcttgcagggacctagggggcctcccggtcctgtgggtggacatggcgatccgggtccaccaggtgctccaggactcgctggtccagcaggccctcagggaccatccggcctgaaaggggaaccaggcgaaactggccccccaggcagaggcctgacaggccctactggtgctgtgggcctccctggacctcctggccctagtggactcgtgggccctcagggctctcccggactgccaggccaagtgggcgagactggaaaacccggggctcccggcagggatggcgcttctggaaaagacggcgataggggcagccctggcgtgcccggtagtccagggctacctggccctgtgggtcccaaaggggagcctggacctacaggcgcaccaggccaggctgtagtggggctgcctggcgctaaaggcgagaagggtgctcctggcggcctggctggcgatctcgttggagaacctggcgccaagggcgaccgtggcttgccaggacctcgcggcgagaaaggcgaagctggcagagctggcgagcctggggacccaggcgaagatggccagaaaggcgctcccggccctaagggattcaagggcgatccgggcgtgggcgtgccaggctctccaggtcctcctggaccacccggtgtcaagggcgatttgggccttcctggcctgccaggggcacctggcgtcgtgggctttcctggacagaccggcccacggggagagatgggacagccaggccccagcggagaaagagggctggctggcccgcctggcagggaaggcataccaggcccattggggcctccaggcccacctggatctgtggggcctcctggcgcctctggactgaaaggcgacaaaggcgatcctggtgtcggcctgccaggcccaagaggcgagaggggagagcccggcatcaggggcgaagatggacggcctggccaagagggccctcggggattgaccggccctcctggatccagaggcgaacggggggagaagggggacgtgggctctgctggcctcaaaggcgacaagggggactccgccgtgattctgggccctcccggacctcggggagctaagggggacatgggagagaggggtccacggggactggatggggacaagggaccacgcggagacaacggcgacccgggggataagggctccaagggcgaacctggcgataagggatccgctggactgcctggcctgaggggcctgctgggacctcaaggacaaccaggcgccgcaggcatccctggcgaccctggatctcctggaaaggacggcgtgcccggcatccgcggagaaaagggggatgtcggcttcatgggccccagggggctgaagggggaaaggggagtgaagggcgcttgcggcctcgatggggaaaagggggacaagggggaggctggccctccaggacgacctggactggctggccacaagggcgaaatgggagagccaggcgtgcccggacagtccggcgctccaggcaaagagggcctgatcggccccaaaggcgatagaggatttgacggccagcctggcccaaagggcgatcaaggcgaaaaaggggagagaggcacccccggcatcggcggctttccaggcccctctggaaacgatggctctgccggcccacctgggccacctggtagtgtgggaccaagaggccccgagggactgcagggacagaaaggcgagagagggccccctggcgagagagttgtgggagcacctggcgttcccggcgcacccggcgaaaggggagaacaaggcagacctggaccagccggaccccgtggggaaaaaggcgaggccgccctgaccgaggacgacatcagaggcttcgtgcggcaagagatgtcccagcactgcgcctgtcagggccagtttatcgcctccggcagcagacccctgccttcctacgctgccgataccgccggctctcagctgcacgctgtgcctgtgctccgggtgtcccacgccgaggaagaggaaagagtccctcctgaggacgacgagtacagcgagtactctgagtattccgtggaagagtaccaggatcccgaggccccttgggacagcgacgacccttgctccctgcctctggatgagggctcctgcaccgcctacaccctgagatggtatcaccgggccgtgacaggctccaccgaggcctgtcaccctttcgtgtatggcggctgcggcggcaacgccaatagattcggcacccgcgaggcctgcgagcggagatgtcctcccagagtggtgcagtcccagggcaccggcacagcccaggactgatagtctagagtcggggcggccggccgctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatccgagtgtcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccctttataaaaccacaatataatggagtgctataatttcaaacagtgtttggtctgctggcagagtggtcattctaacagcagtcacagtagagtagaaataagactgcagtatatctaaggcaaaaagctgaggtttcaggagcttgaaggtaaagaggaagaaagaaatgggaatgggaattggaaagacaaatatcgttaagagaaaattgcttttaggagaggggaaagaatctatgtgtacttaagactatggaatcaatcccatttaagctgggaaactagtttcatatataactaataaattttatttacagaatatctatttacctgatctaggcttcaagccaaagggactgtgtgaaaaaccatcagttctgtcatattcctaaaaaaaaattaaaaagttaaaaataaataaataataaaacttcttttctttcaaaataatcaaggtgcttattcacatccattccaatttggggaaatacttattttcctatgattagcgaagagaaaagtaacttgcatttcaattcaagttgatacatgtcacttttaagaggtcaactaatatttgctagttgagctaaccatataggctttaaatactttcatagtagaaagaaaatgaaaatcattagtgaactgtataaaatagatcatactttttgaaagaatcagactgaagtttccgaaaaaaagaagtaagctagcttcaatgaaaaggtaagtgaatttagcatttactcagcatctactatggacttaacacctaacagtagataatctgaaggcaaacatatttgtatagggactgcagaatgatagatgataaatatcatctcttctatttgaatgaatattttttcaaatctttcacacacagtggtttgctatggaaagatttgtagtacattaaacaaatctgaagatggagttagaaagctagcttaggctatgttttgagcacaacatataatttctctgtgattgtttcttcatctttcaaatgaggttactgtgaagattaaatgagataactaaatgatgataaaataatgtaatcttagcagcaccttatttaatctgtgcaacaactctgtgaagtgagtagggctcagcttcagtcacttctctgccatttattaactaagatagtttggaaagttacccatctcttcagctgtaaaatgatgaggatcatacctattttatggggctgcttttaggtacaaatatacaggcaagcactttgttaatactaaagcattacaccaattagttttactcttttccattcacacatgaaattaatgtaatcagaattctgtagattacctaaatcttctgttaacacgtgatatgcagttcaggttaaatgtcagttgagttaccaaagcacatacatactcaccaccctatccaaatctacaagcctcccagtttgtcttcactattttggttaaattaatatgaattcctagatgaaaatttcactgatccaaatgaaataaaaaatatattacaaaactcacacctgtaatctcaacattttgggaggccaaggcaggtagatcacttgaggccaggagttcaagaccagcctgatcaacatggtgaaaccctgtctctactaaaaatacaaaaattagccaggtgtggtggcatgtgcctgtagtcctacctactcgggaggctgaggcacaagaatcgcttgaatgtgggaggtggaggttgcagtgacctgagatcgtgccactgcactccagcctaggcaacagagtgagatcatgtgtcatatatatatatatatatatatatatatatatatatatatacacacacacacacatatatatatacacatatatatacgtatatatatatatgtatatatatacatatatatacatatatatatatacgtatatatatacgtatatatatatcaatgtaaattatttgggaaatttggtatgaatagtcttccctgtgaacacagatcataaaatcatatatcaagcagacaaataagtagtagtcacttatatgcttatacttgtaacttaaagtaaaagaattacaaaagcatatgacaaagactaattttaagatatcctaatttaaattgttttctaaaagtgtgtataccattttacctatcatatgaataatttagaaacatgtttataaaattaatgtccaaatccattcaaaagttttgtaatgcagatcacccacaacaacaaagaatcctagcctattaaaaaagcaacaccacctacatataatgaaatattagcagcatctatgtaaccaaagttacacagtgaatttgggccatccaacactttgagcaaagtgttgaattcatcaaatgaatgtgtaatcatttacttactaatgccaatacactttaaggtaatcttaagtagaagagatagagtttagaattttttaaatttatctcttgttgtaaagcaatagacttgaataaataaattagaagaatcagtcattcaagccaccagagtatttgatcgagatttcacaaactctaactttctgatacccattctcccaaaaacgtgtaacctcctgtcgataggaacaacccactgcagggatgtttctcgtggaaaaaggaaatttcttttgcattggtttcagacctaactggttacaagaaaaaccaaaggccattgcacaatgctgaagtacttttttcaaatttaaaatttgaaagttgttcttaaaatctatcatttattttaaaatacggatgaatgagaaagcatagatttgataaagtgaattcttttctgcaatctacagacacttccaaaaatcactacagacactacagacactacagaaaatcataaataaacaagtgctagtatcaatatttttaccaaaaaatggcattcttagaattttttataggctagaaggtttgtacaaactaatctgccacggattttaaaatatgagtgaataaattatattgcaaaaaaaatcaggttacagagaactggcaaggaagactcttatgtaaaacacagaaaacatacaaaacgtatttttaagacaaataaaaacagaacttgtacctcagatgatactggagattgtgttgacatattagcattatcactgtcttgctaaaacataaaaataaaaagatggaagatgaaattacaatacaaatgatgatttaaacatataaaaggaaaataaaaattgttctgaccaactactaaaggaagacctactaaagatatgccatccagcacattgccactctacatgtggtctgtaaaccagcagcatagggatccgtcgatcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagctgcggcccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggcaatggatgcgattgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtggggatcgggagatgggggaggctaactgagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctcta

SEQ ID NO: 28 - DNA, 1602 bp, synthetic construct
  source 1..1602: mol_type = other DNA; note = Artificial sequence: Plasmid insertion of sequence encoding alpha polypeptide 1 of human P4H
atgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgagccgagctacagtacatgaccctgagactggaaaattgaccacagcacagtacagagtatctaagagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaa

SEQ ID NO: 29 - DNA, 10220 bp, synthetic construct
  source 1..10220: mol_type = other DNA; note = Artificial sequence: Vector for alpha polypeptide 1 of human P4H
gtaccttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttggtatcgatgtaccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgacctcgaggagaagttccccaactttcccgcctctcagcctttgaaagaaagaaaggggagggggcaggccgcgtgcagccgcgagcggtgctgggctccggctccaattccccatctcagtcgttcccaaagtcctcctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgtgtgtcggccggggccactaggcgctcactgttctctccctccgcgcagccgagccacatcgctcagacaccaagcttaccgccaccatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgagccgagctacagtacatgaccctgagactggaaaattgaccacagcacagtacagagtatctaagagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgataagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataatatatggtagggttcatagccagagtaacctttttttttaatttttattttattttatttttgagtcgcgagtgtcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccctttataaaaccacaatataatggagtgctataatttcaaacagtgtttggtctgctggcagagtggtcattctaacagcagtcacagtagagtagaaataagactgcagtatatctaaggcaaaaagctgaggtttcaggagcttgaaggtaaagaggaagaaagaaatgggaatgggaattggaaagacaaatatcgttaagagaaaattgcttttaggagaggggaaagaatctatgtgtacttaagactatggaatcaatcccatttaagctgggaaactagtttcatatataactaataaattttatttacagaatatctatttacctgatctaggcttcaagccaaagggactgtgtgaaaaaccatcagttctgtcatattcctaaaaaaaaattaaaaagttaaaaataaataaataataaaacttcttttctttcaaaataatcaaggtgcttattcacatccattccaatttggggaaatacttattttcctatgattagcgaagagaaaagtaacttgcatttcaattcaagttgatacatgtcacttttaagaggtcaactaatatttgctagttgagctaaccatataggctttaaatactttcatagtagaaagaaaatgaaaatcattagtgaactgtataaaatagatcatactttttgaaagaatcagactgaagtttccgaaaaaaagaagtaagctagcttcaatgaaaaggtaagtgaatttagcatttactcagcatctactatggacttaacacctaacagtagataatctgaaggcaaacatatttgtatagggactgcagaatgatagatgataaatatcatctcttctatttgaatgaatattttttcaaatctttcacacacagtggtttgctatggaaagatttgtagtacattaaacaaatctgaagatggagttagaaagctagcttaggctatgttttgagcacaacatataatttctctgtgattgtttcttcatctttcaaatgaggttactgtgaagattaaatgagataactaaatgatgataaaataatgtaatcttagcagcaccttatttaatctgtgcaacaactctgtgaagtgagtagggctcagcttcagtcacttctctgccatttattaactaagatagtttggaaagttacccatctcttcagctgtaaaatgatgaggatcatacctattttatggggctgcttttaggtacaaatatacaggcaagcactttgttaatactaaagcattacaccaattagttttactcttttccattcacacatgaaattaatgtaatcagaattctgtagattacctaaatcttctgttaacacgtgatatgcagttcaggttaaatgtcagttgagttaccaaagcacatacatactcaccaccctatccaaatctacaagcctcccagtttgtcttcactattttggttaaattaatatgaattcctagatgaaaatttcactgatccaaatgaaataaaaaatatattacaaaactcacacctgtaatctcaacattttgggaggccaaggcaggtagatcacttgaggccaggagttcaagaccagcctgatcaacatggtgaaaccctgtctctactaaaaatacaaaaattagccaggtgtggtggcatgtgcctgtagtcctacctactcgggaggctgaggcacaagaatcgcttgaatgtgggaggtggaggttgcagtgacctgagatcgtgccactgcactccagcctaggcaacagagtgagatcatgtgtcatatatatatatatatatatatatatatatatatatatatacacacacacacacatatatatatacacatatatatacgtatatatatatatgtatatatatacatatatatacatatatatatatacgtatatatatacgtatatatatatcaatgtaaattatttgggaaatttggtatgaatagtcttccctgtgaacacagatcataaaatcatatatcaagcagacaaataagtagtagtcacttatatgcttatacttgtaacttaaagtaaaagaattacaaaagcatatgacaaagactaattttaagatatcctaatttaaattgttttctaaaagtgtgtataccattttacctatcatatgaataatttagaaacatgtttataaaattaatgtccaaatccattcaaaagttttgtaatgcagatcacccacaacaacaaagaatcctagcctattaaaaaagcaacaccacctacatataatgaaatattagcagcatctatgtaaccaaagttacacagtgaatttgggccatccaacactttgagcaaagtgttgaattcatcaaatgaatgtgtaatcatttacttactaatgccaatacactttaaggtaatcttaagtagaagagatagagtttagaattttttaaatttatctcttgttgtaaagcaatagacttgaataaataaattagaagaatcagtcattcaagccaccagagtatttgatcgagatttcacaaactctaactttctgatacccattctcccaaaaacgtgtaacctcctgtcgataggaacaacccactgcagggatgtttctcgtggaaaaaggaaatttcttttgcattggtttcagacctaactggttacaagaaaaaccaaaggccattgcacaatgctgaagtacttttttcaaatttaaaatttgaaagttgttcttaaaatctatcatttattttaaaatacggatgaatgagaaagcatagatttgataaagtgaattcttttctgcaatctacagacacttccaaaaatcactacagacactacagacactacagaaaatcataaataaacaagtgctagtatcaatatttttaccaaaaaatggcattcttagaattttttataggctagaaggtttgtacaaactaatctgccacggattttaaaatatgagtgaataaattatattgcaaaaaaaatcaggttacagagaactggcaaggaagactcttatgtaaaacacagaaaacatacaaaacgtatttttaagacaaataaaaacagaacttgtacctcagatgatactggagattgtgttgacatattagcattatcactgtcttgctaaaacataaaaataaaaagatggaagatgaaattacaatacaaatgatgatttaaacatataaaaggaaaataaaaattgttctgaccaactactaaaggaagacctactaaagatatgccatccagcacattgccactctacatgtggtctgtaaaccagcagcatagggatccactagagcttgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgg

SEQ ID NO: 30 - DNA, 1524 bp, synthetic construct
  source 1..1524: mol_type = other DNA; note = Artificial sequence: Plasmid insertion of sequence encoding beta polypeptide of human P4H
atgctgcgccgcgctctgctgtgcctggccgtggccgccctggtgcgcgccgacgcccccgaggaggaggaccacgtcctggtgctgcggaaaagcaacttcgcggaggcgctggcggcccacaagtacctgctggtggagttctatgccccttggtgtggccactgcaaggctctggcccctgagtatgccaaagccgctgggaagctgaaggcagaaggttccgagatcaggttggccaaggtggacgccacggaggagtctgacctggcccagcagtacggcgtgcgcggctatcccaccatcaagttcttcaggaatggagacacggcttcccccaaggaatatacagctggcagagaggctgatgacatcgtgaactggctgaagaagcgcacgggcccggctgccaccaccctgcctgacggcgcagctgcagagtccttggtggagtccagcgaggtggctgtcatcggcttcttcaaggacgtggagtcggactctgccaagcagtttttgcaggcagcagaggccatcgatgacataccatttgggatcacttccaacagtgacgtgttctccaaataccagctcgacaaagatggggttgtcctctttaagaagtttgatgaaggccggaacaactttgaaggggaggtcaccaaggagaacctgctggactttatcaaacacaaccagctgccccttgtcatcgagttcaccgagcagacagccccgaagatttttggaggtgaaatcaagactcacatcctgctgttcttgcccaagagtgtgtctgactatgacggcaaactgagcaacttcaaaacagcagccgagagcttcaagggcaagatcctgttcatcttcatcgacagcgaccacaccgacaaccagcgcatcctcgagttctttggcctgaagaaggaagagtgcccggccgtgcgcctcatcaccctggaggaggagatgaccaagtacaagcccgaatcggaggagctgacggcagagaggatcacagagttctgccaccgcttcctggagggcaaaatcaagccccacctgatgagccaggagctgccggaggactgggacaagcagcctgtcaaggtgcttgttgggaagaactttgaagacgtggcttttgatgagaaaaaaaacgtctttgtggagttctatgccccatggtgtggtcactgcaaacagttggctcccatttgggataaactgggagagacgtacaaggaccatgagaacatcgtcatcgccaagatggactcgactgccaacgaggtggaggccgtcaaagtgcacagcttccccacactcaagttctttcctgccagtgccgacaggacggtcatcgattacaacggggaacgcacgctggatggttttaagaaattcctggagagcggtggccaggatggggcaggggatgatgacgatctcgaggacctggaagaagcagaggagccagacatggaggaagacgatgatcagaaagctgtgaaagatgaactg

SEQ ID NO: 31 - DNA, 10148 bp, synthetic construct
  source 1..10148: mol_type = other DNA; note = Artificial sequence: Vector for beta polypeptide of P4H
gtaccttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttggtatcgatgtaccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgacctcgaggagaagttccccaactttcccgcctctcagcctttgaaagaaagaaaggggagggggcaggccgcgtgcagccgcgagcggtgctgggctccggctccaattccccatctcagtcgttcccaaagtcctcctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgtgtgtcggccggggccactaggcgctcactgttctctccctccgcgcagccgagccacatcgctcagacaccaagcttaccgccaccatgctgcgccgcgctctgctgtgcctggccgtggccgccctggtgcgcgccgacgcccccgaggaggaggaccacgtcctggtgctgcggaaaagcaacttcgcggaggcgctggcggcccacaagtacctgctggtggagttctatgccccttggtgtggccactgcaaggctctggcccctgagtatgccaaagccgctgggaagctgaaggcagaaggttccgagatcaggttggccaaggtggacgccacggaggagtctgacctggcccagcagtacggcgtgcgcggctatcccaccatcaagttcttcaggaatggagacacggcttcccccaaggaatatacagctggcagagaggctgatgacatcgtgaactggctgaagaagcgcacgggcccggctgccaccaccctgcctgacggcgcagctgcagagtccttggtggagtccagcgaggtggctgtcatcggcttcttcaaggacgtggagtcggactctgccaagcagtttttgcaggcagcagaggccatcgatgacataccatttgggatcacttccaacagtgacgtgttctccaaataccagctcgacaaagatggggttgtcctctttaagaagtttgatgaaggccggaacaactttgaaggggaggtcaccaaggagaacctgctggactttatcaaacacaaccagctgccccttgtcatcgagttcaccgagcagacagccccgaagatttttggaggtgaaatcaagactcacatcctgctgttcttgcccaagagtgtgtctgactatgacggcaaactgagcaacttcaaaacagcagccgagagcttcaagggcaagatcctgttcatcttcatcgacagcgaccacaccgacaaccagcgcatcctcgagttctttggcctgaagaaggaagagtgcccggccgtgcgcctcatcaccctggaggaggagatgaccaagtacaagcccgaatcggaggagctgacggcagagaggatcacagagttctgccaccgcttcctggagggcaaaatcaagccccacctgatgagccaggagctgccggaggactgggacaagcagcctgtcaaggtgcttgttgggaagaactttgaagacgtggcttttgatgagaaaaaaaacgtctttgtggagttctatgccccatggtgtggtcactgcaaacagttggctcccatttgggataaactgggagagacgtacaaggaccatgagaacatcgtcatcgccaagatggactcgactgccaacgaggtggaggccgtcaaagtgcacagcttccccacactcaagttctttcctgccagtgccgacaggacggtcatcgattacaacggggaacgcacgctggatggttttaagaaattcctggagagcggtggccaggatggggcaggggatgatgacgatctcgaggacctggaagaagcagaggagccagacatggaggaagacgatgatcagaaagctgtgaaagatgaactgtgataatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataatatatggtagggttcatagccagagtaacctttttttttaatttttattttattttatttttgagtcgcgagtgtcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccctttataaaaccacaatataatggagtgctataatttcaaacagtgtttggtctgctggcagagtggtcattctaacagcagtcacagtagagtagaaataagactgcagtatatctaaggcaaaaagctgaggtttcaggagcttgaaggtaaagaggaagaaagaaatgggaatgggaattggaaagacaaatatcgttaagagaaaattgcttttaggagaggggaaagaatctatgtgtacttaagactatggaatcaatcccatttaagctgggaaactagtttcatatataactaataaattttatttacagaatatctatttacctgatctaggcttcaagccaaagggactgtgtgaaaaaccatcagttctgtcatattcctaaaaaaaaattaaaaagttaaaaataaataaataataaaacttcttttctttcaaaataatcaaggtgcttattcacatccattccaatttggggaaatacttattttcctatgattagcgaagagaaaagtaacttgcatttcaattcaagttgatacatgtcacttttaagaggtcaactaatatttgctagttgagctaaccatataggctttaaatactttcatagtagaaagaaaatgaaaatcattagtgaactgtataaaatagatcatactttttgaaagaatcagactgaagtttccgaaaaaaagaagtaagctagcttcaatgaaaaggtaagtgaatttagcatttactcagcatctactatggacttaacacctaacagtagataatctgaaggcaaacatatttgtatagggactgcagaatgatagatgataaatatcatctcttctatttgaatgaatattttttcaaatctttcacacacagtggtttgctatggaaagatttgtagtacattaaacaaatctgaagatggagttagaaagctagcttaggctatgttttgagcacaacatataatttctctgtgattgtttcttcatctttcaaatgaggttactgtgaagattaaatgagataactaaatgatgataaaataatgtaatcttagcagcaccttatttaatctgtgcaacaactctgtgaagtgagtagggctcagcttcagtcacttctctgccatttattaactaagatagtttggaaagttacccatctcttcagctgtaaaatgatgaggatcatacctattttatggggctgcttttaggtacaaatatacaggcaagcactttgttaatactaaagcattacaccaattagttttactcttttccattcacacatgaaattaatgtaatcagaattctgtagattacctaaatcttctgttaacacgtgatatgcagttcaggttaaatgtcagttgagttaccaaagcacatacatactcaccaccctatccaaatctacaagcctcccagtttgtcttcactattttggttaaattaatatgaattcctagatgaaaatttcactgatccaaatgaaataaaaaatatattacaaaactcacacctgtaatctcaacattttgggaggccaaggcaggtagatcacttgaggccaggagttcaagaccagcctgatcaacatggtgaaaccctgtctctactaaaaatacaaaaattagccaggtgtggtggcatgtgcctgtagtcctacctactcgggaggctgaggcacaagaatcgcttgaatgtgggaggtggaggttgcagtgacctgagatcgtgccactgcactccagcctaggcaacagagtgagatcatgtgtcatatatatatatatatatatatatatatatatatatatatacacacacacacacatatatatatacacatatatatacgtatatatatatatgtatatatatacatatatatacatatatatatatacgtatatatatacgtatatatatatcaatgtaaattatttgggaaatttggtatgaatagtcttccctgtgaacacagatcataaaatcatatatcaagcagacaaataagtagtagtcacttatatgcttatacttgtaacttaaagtaaaagaattacaaaagcatatgacaaagactaattttaagatatcctaatttaaattgttttctaaaagtgtgtataccattttacctatcatatgaataatttagaaacatgtttataaaattaatgtccaaatccattcaaaagttttgtaatgcagatcacccacaacaacaaagaatcctagcctattaaaaaagcaacaccacctacatataatgaaatattagcagcatctatgtaaccaaagttacacagtgaatttgggccatccaacactttgagcaaagtgttgaattcatcaaatgaatgtgtaatcatttacttactaatgccaatacactttaaggtaatcttaagtagaagagatagagtttagaattttttaaatttatctcttgttgtaaagcaatagacttgaataaataaattagaagaatcagtcattcaagccaccagagtatttgatcgagatttcacaaactctaactttctgatacccattctcccaaaaacgtgtaacctcctgtcgataggaacaacccactgcagggatgtttctcgtggaaaaaggaaatttcttttgcattggtttcagacctaactggttacaagaaaaaccaaaggccattgcacaatgctgaagtacttttttcaaatttaaaatttgaaagttgttcttaaaatctatcatttattttaaaatacggatgaatgagaaagcatagatttgataaagtgaattcttttctgcaatctacagacacttccaaaaatcactacagacactacagacactacagaaaatcataaataaacaagtgctagtatcaatatttttaccaaaaaatggcattcttagaattttttataggctagaaggtttgtacaaactaatctgccacggattttaaaatatgagtgaataaattatattgcaaaaaaaatcaggttacagagaactggcaaggaagactcttatgtaaaacacagaaaacatacaaaacgtatttttaagacaaataaaaacagaacttgtacctcagatgatactggagattgtgttgacatattagcattatcactgtcttgctaaaacataaaaataaaaagatggaagatgaaattacaatacaaatgatgatttaaacatataaaaggaaaataaaaattgttctgaccaactactaaaggaagacctactaaagatatgccatccagcacattgccactctacatgtggtctgtaaaccagcagcatagggatccactagagcttgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgg

SEQ ID NO: 32 - DNA, 1254 bp, synthetic construct
  source 1..1254: mol_type = other DNA; note = Artificial sequence: Plasmid insertion of sequence encoding HSP 47
atgcgctcgcttctgctcctgtcggccttctgcctcctggaggccgcgctggctgccgaggtcaagaagccggccgccgcggccgcgcccggcacggccgagaagctgtcgccgaaggcggccaccctggctgagcgctccgcgggcctcgccttctcactgtaccaggcgatggcaaaggaccaggccgtggaaaacatcctggtcagccccgtcgtggttgcctccagcctgggcctggtctccctgggcggcaaggccaccacggccagtcaggccaaggccgtcctctcggcggagcagctccgcgacgaggaagtgcacgccggactgggcgagctgctgcggtccctgagcaactccactgctcggaacgttacctggaagctgggatcgaggctctacggcccgtcctccgtctcgttcgcggacgacttcgtccgcagctcgaagcagcactacaactgcgagcactccaagatcaacttccgcgacaagcggtccgcgctgcagtccatcaacgagtgggcggcccagacgaccgacgggaagctgccggaggtcaccaaagacgtcgagcgcacggacggagccctcctggtcaacgccatgttcttcaaaccgcactgggacgagaagttccatcacaagatggtggacaacaggggtttcatggtgacgcgctcctacaccgtgggagtcatgatgatgcaccgcacgggcctgtacaactactacgacgacgagaaggaaaagctccagatcgtggagatgccgctggcccacaagctgtcctcgctcattatcctcatgccccaccatgtcgagcccctggagcgcctcgaaaaactcctgaccaaggaacagctgaagatctggatgggcaagatgcagaaaaaggccgtcgccatctccctccccaagggcgtggtcgaagtcactcacgacctgcagaagcacctggcgggcctgggactgacggaggccatcgacaagaacaaggctgacctgtcccgcatgtccgggaagaaggacttgtacctggcctccgtcttccacgcgaccgccttcgaactggacaccgatggcaacccgttcgaccaggacatctacgggcgcgaagagctgcgctcccccaagctgttctacgccgaccacccgttcatcttcctggtccgcgatacgcagtccggcagcctgttgttcatcggccggctcgtgcggccgaagggcgacaagatgcgggacgaactc

SEQ ID NO: 33 - DNA, 45 bp, synthetic construct
  source 1..45: mol_type = other DNA; note = Artificial sequence: PCR primer
taccgccaccatgatctggtatatattaattataggaattctgct

SEQ ID NO: 34 - DNA, 48 bp, synthetic construct
  source 1..48: mol_type = other DNA; note = Artificial sequence: PCR primer
tcatggccggccgccccgacttatcattccaattctgacaacgtacaa

SEQ ID NO: 35 - DNA, 36 bp, synthetic construct
  source 1..36: mol_type = other DNA; note = Artificial sequence: PCR primer
tcccaagcttaccgccaccatgctgcgccgcgctct

SEQ ID NO: 36 - DNA, 41 bp, synthetic construct
  source 1..41: mol_type = other DNA; note = Artificial sequence: PCR primer
ctagtctagattatcacagttcatctttcacagctttctga

SEQ ID NO: 37 - DNA, 37 bp, synthetic construct
  source 1..37: mol_type = other DNA; note = Artificial sequence: PCR primer
tcccaagcttaccgccaccatggcggcggccaccgga

SEQ ID NO: 38 - DNA, 37 bp, synthetic construct
  source 1..37: mol_type = other DNA; note = Artificial sequence: PCR primer
ctagtctagattatcacttggggccagagaagggggt

SEQ ID NO: 39 - DNA, 33 bp, synthetic construct
  source 1..33: mol_type = other DNA; note = Artificial sequence: PCR primer
taccgccaccatgctttctgaaagcagctcctt

SEQ ID NO: 40 - DNA, 38 bp, synthetic construct
  source 1..38: mol_type = other DNA; note = Artificial sequence: PCR primer
ctagtctagattagtcattgtcagaaccatttggaggt

(19) United States
(12) Patent Application Publication
(10) Pub. No.: US 2026/0209315 A1
(43) Pub. Date: Jul. 23, 2026

(54) SYSTEMS AND METHODS FOR PRODUCING COLLAGEN 7 COMPOSITIONS

(71) Applicant: Phoenix Tissue Repair, Inc., Boston, MA (US)

(72) Inventors: Mark DESOUZA, Berkeley, CA (US); Malini VISWANATHAN, Acton, MA (US); Pierre-Alain GIROD, Geneva (CH); Alexandre REGAMEY, Geneva (CH); Valerie LE FOURN, Geneva (CH); David CALABRESE, Geneva (CH); John GILL, Lexington, MA (US); Megan GOTTLIEB, Lexington, MA (US); Amie LUNDQUIST, Lexington, MA (US); Anna TCHOUDAKOVA, Lexington, MA (US); George ENRIQUEZ, Lexington, MA (US); Rahul CHELIKANI, Lexington, MA (US); Thomas GAGLIARDI, Lexington, MA (US); Tatiana LAIVINS, Hingham, MA (US); Hang YUAN, Lexington, MA (US)

(21) Appl. No.: 19/633,753

(22) Filed: Mar. 30, 2026

Related U.S. Application Data
(63) Continuation of application No. 17/598,029, filed on Mar. 27, 2020.
(60) Provisional application No. 62/824,671, filed on Mar. 27, 2019.

(51) Int. Cl. C07K 14/78; A61K 38/00; C12N 9/02

(52) U.S. Cl. CPC C07K 14/78; C12N 9/0071; C12Y 114/11002; A61K 38/00

(57) ABSTRACT

The present disclosure provides production systems and host cells to produce collagen 7 compositions comprising recombinant collagen 7 and/or functional variants thereof. The host cells are genetically engineered to stably express rCol7 and functional variants thereof. The collagen 7 composition can be used to restore collagen 7 levels in a subject in need, and for preventing, preventing the progression of, alleviating, and delaying the on-set of a skin condition, e.g., skin wound associated with dystrophic epidermolysis bullosa (DEB).

### REFERENCE TO RELATED DISCLOSURES

The present application is a Continuation application of U.S. patent application Ser. No. 17/598,029 filed Sep. 24, 2021, which is a U.S. National Phase entry of PCT Patent Application No.: PCT/US2020/025129, filed Mar. 27, 2020, which claims the benefit of, and relies on the filing date of, U.S. provisional patent application No. 62/824,671, filed Mar. 27, 2019, the contents of which are incorporated herein by reference in their entirety.

### FIELD OF THE DISCLOSURE

The present disclosure relates to production systems, engineered host cells, and methods for producing collagen 7 compositions comprising recombinant human collagen 7 and/or functional variants thereof.

### REFERENCE TO THE SEQUENCE LISTING

The instant application contains a Sequence Listing which has been submitted electronically in XML format and is hereby incorporated by reference in its entirety. Said XML copy, created on Mar. 30, 2026, is named “51624-010003_Sequence_Listing_3_30_26” and is 152,724 bytes in size.

### BACKGROUND OF THE DISCLOSURE

Collagen 7 (Collagen Type VII), is an important component of the skin found in the epidermal basement membrane zone (BMZ), the two-layer membrane located between the epidermal and underlying dermal layers of the skin. Anchoring fibrils composed of collagen 7 connect the epidermal basement membrane to the papillary dermis, holding the epidermal and dermal layers of the skin together, providing structure and stability.

Collagen 7 is a homotrimer consisting of three identical alpha-chain polypeptides. The collagen 7 alpha chain polypeptide is encoded by the COL7A1 gene and is primarily expressed and synthesized by keratinocytes and fibroblasts. Alterations in collagen 7 have been linked to several skin disorders, such as epidermolysis bullosa (EB) and autoimmune diseases caused by autoantibodies against collagen 7 protein, such as epidermolysis bullosa acquisita (EBA), bullous pemphigoid, cicatricial pemphigoid, paraneoplastic pemphigus, pemphigus vulgaris, chronic bullous disease of childhood (CBDC), and systemic sclerosis.

Mutations in the COL7A1 gene can result in a form of EB known as dystrophic epidermolysis bullosa (DEB), a rare pediatric disease presenting with extremely fragile and incurable blistering skin, deformed limbs, esophageal strictures, numerous other co-morbidities, and early death. Based on inheritance pattern, DEB can be categorized as autosomal dominant DEB (DDEB) or recessive DEB (RDEB); the latter resulting in the most severe form of DEB. The incidence of all types of DEB is estimated to be 6.5 per million newborns in the United States, while the more severe autosomal recessive form affects about 1 per million newborns.

Other common symptoms of skin disorders associated with deficiencies in collagen 7 include urticarial eruption of the skin (hives), bullae (blistering) on the skin (including both epidermal and subepidermal blistering), chronic skin wounds, severe and erosive lesions of the mucous membranes, including the oral or rectal mucosa, conjunctiva, nasoharynx, larynx, and esophagus, and pain and scarring of the skin.

Treatment strategies for these severe skin conditions and disorders (e.g., DEB) focus on restoring functional collagen 7. Exemplary strategies include topical and/or local administration of collagen 7, gene therapy targeting the COL7A1 gene, cell therapy such as transplant of fibroblast cells for expression of collagen 7, and collagen 7 replacement therapy such as by systemic administration of collagen 7 protein.

The present disclosure provides production systems, engineered host cells and methods for producing collagen 7 compositions for restoring functional collagen 7 in a subject. In some embodiments, the collagen 7 compositions may be used for treatment of a skin condition, such as one caused by collagen 7 deficiency and/or other defects in the basement membrane zone (BMZ) of the skin.

### SUMMARY OF THE DISCLOSURE

The present disclosure provides production systems for producing collagen 7 compositions comprising human recombinant collagen 7 (rCol7) and/or functional variants thereof, wherein the production system comprises host cells modified for expressing human rCol7 and/or functional variants thereof. Preferably, the engineered host cells of the production system are transformed to express human rCol7, or functional variants thereof, and at least one protein that can increase the expression of rCol7 and/or functional variants thereof in host cells, including a prolidase (also known as proline hydrolase), prolyl 4-hydroxylase, consisting of an alpha polypeptide (subunit A) and a beta polypeptide (subunit B), C1GALT1 Specific chaperone 1 (COMSC) and/or a heat shock protein (HSP) (e.g., HSP 47). Preferably, the host cells are engineered to express human prolyl 4-hydroxylase, comprising both the alpha and beta polypeptides (e.g., hP4HA1 and hP4HB) only, or in combination with HSP 47, to increase the expression of rCol7.

In some embodiments, the production system may comprise a plurality of homogenous engineered host cells derived from a single cell clone expressing human rCol7, or a functional variant thereof. In other embodiments, the production system may comprise a plurality of heterogenous engineered host cells derived from more than one cell clones expressing human rCol7 and/or functional variants thereof.

In some embodiments, the engineered host cells comprise at least one first exogenous polynucleotide that encodes human rCol7, or a functional variant thereof, and at least one exogenous polynucleotide encoding a protein that can increase the expression of rCol7 or functional variants thereof, such as prolidase (PEPD), prolyl 4-hydroxylase (P4H), C1GALT1 specific chaperone 1 (COMSC), and heat shock protein 47 (HSP 47). In some embodiments, the engineered host cells may further comprise a second polynucleotide encoding rCol7 or a functional variant thereof. The first and second polynucleotides encoding the rCol7 and functional variants thereof may comprise either the identical nucleic acid sequence, or different nucleic acid sequences. In some embodiments, the polynucleotides encoding rCol7 comprise codon-optimized nucleic acid sequences.

In one preferred embodiment, the engineered host cells comprise at least one first exogenous polynucleotide encoding rCol7, or a functional variant thereof, an exogenous polynucleotide encoding the alpha polypeptide (subunit A) of human prolyl 4-hydroxylase (P4HA), and an exogenous polynucleotide encoding the beta polypeptide (subunit B) of human prolyl 4-hydroxylase (P4HB). Optionally, the engineered host cells may further comprise a second exogenous polynucleotide encoding rCol7, or a functional variant thereof. In some examples, the first rCol7 encoding polynucleotide and the second rCol7 encoding polynucleotide may have the same nucleic acid sequence. In other examples, the two rCol7 encoding polynucleotides have different nucleic acid sequences.

In some embodiments, the engineered host cells comprise one first and one second exogenous polynucleotides encoding rCol7, or a functional variant thereof, an exogenous polynucleotide encoding the alpha polypeptide (subunit A) of human prolyl 4-hydroxylase, an exogenous polynucleotide encoding the beta polypeptide (subunit B) of human prolyl 4-hydroxylase, and an exogenous polynucleotide encoding HSP 47.

In some embodiments, the engineered host cells are transformed with an expression vector comprising a polynucleotide encoding rCol7, or a functional variant thereof, and at least one expression vector comprising a polynucleotide that encodes a protein that can increase the expression of rCol7 or functional variants thereof, wherein the protein may be a prolidase (PEPD), prolyl 4-hydroxylase (P4H), C1GALT1 Specific chaperone 1 (COMSC) or heat shock protein 47 (HSP 47).

In one preferred embodiment, the engineered host cells are transformed with an expression vector comprising a first polynucleotide encoding human rCol7, or a functional variant thereof, an expression vector comprising a polynucleotide encoding the alpha polypeptide (subunit A) of human prolyl 4-hydroxylase, and an expression vector comprising a polynucleotide encoding the beta polypeptide (subunit B) of human prolyl 4-hydroxylase. Optionally, the engineered host cells are further transformed with an expression vector comprising a second polynucleotide encoding human rCol7, or a functional variant thereof. In some examples, such engineered host cells are further transformed with a vector expressing a polynucleotide encoding HSP 47.

In some embodiments, the engineered host cells are transformed with an expression vector comprising a polynucleotide encoding rCol7, or a functional variant thereof, wherein the same expression vector comprises a polynucleotide encoding the alpha polypeptide (subunit A) of human prolyl 4-hydroxylase and a polynucleotide encoding the beta polypeptide (subunit B) of human prolyl 4-hydroxylase.

In some embodiments, the engineered host cells are transformed with an expression vector comprising a polynucleotide encoding rCol7, or a functional variant thereof, wherein the same expression vector comprises a polynucleotide encoding the alpha polypeptide (subunit A) of human prolyl 4-hydroxylase and a polynucleotide encoding the beta polypeptide (subunit B) of human prolyl 4-hydroxylase and a polynucleotide encoding HSP 47.

In some embodiments, the engineered host cells are mammalian cells, e.g., human, mouse, rat, or Chinese hamster cells (CHO). In one preferred embodiment, the cells are mammalian cells derived from a CHO cell line.

The engineered host cells may be cultured under a protein production condition that is serum free. The protein production condition may comprise addition of one or more agents such as nutrients and/or selectable agents.

In another aspect of the present disclosure, methods for producing a collagen 7 composition are provided, the methods comprising (1) introducing into mammalian cells at least one exogenous polynucleotide encoding rCol7, or a functional variant thereof, an exogenous polynucleotide encoding the alpha polypeptide (subunit A) of prolyl 4-hydroxylase, and an exogenous polynucleotide encoding the beta polypeptide (subunit B) of prolyl 4-hydroxylase; (2) selecting stable monoclonal transformants of said mammalian cells by isolating those transformants which express a selectable marker at a sufficient level to allow their survival when grown in the presence of a selecting agent; (3) culturing the transformants under growth conditions which allow expression of rCol7, or the functional variant thereof, and other polypeptides to produce the protein composition; and (4) harvesting the cell culture media and purifying the protein composition.

In some embodiments, the method further comprises introducing into the mammalian cells of step (1) an exogenous polynucleotide encoding HSP 47.

In some embodiments, the growth conditions are serum free and comprise addition of one or more agents such as nutrients and/or selectable agents.

Another aspect of the present disclosure relates to collagen 7 compositions comprising human rCol7 and/or functional variants thereof, and pharmaceutical compositions or formulations thereof, comprising a collagen 7 composition and at least one pharmaceutically acceptable carrier. In some embodiments, the pharmaceutical composition or formulation is suitable for systemic administration to a subject in need. The subject may bear a deficiency in the COL7A1 gene. In some examples, the subject may be diagnosed with RDEB.

In some embodiments, the collagen 7 compositions produced by the present production system may comprise a naturally occurring human collagen 7 protein comprising a polypeptide of SEQ ID NO.: 1, a functional variant thereof, or a combination of naturally occurring collagen 7 protein and functional variants thereof.

In some embodiments, the collagen 7 compositions produced by the present production system, host cells and methods may be used to restore collagen 7 to functional levels in a subject in need.

In yet another aspect, the present disclosure provides methods for preventing, alleviating and inhibiting the progress of a skin condition in a subject; the methods comprising administering to the subject a pharmaceutical composition comprising a collagen 7 composition containing rCol7 and/or functional variants thereof. In some embodiments, the condition is a skin condition associated with a mutation in the COL7A1 gene, such as RDEB.

### BRIEF SUMMARY OF THE DRAWINGS

FIG. 1 is a gel image of a western blot of cells from day 6 in culture for the B1STBSTb cell clone first round candidates (at 32° C.).

FIG. 2A is a gel image of a western blot of cells from day 6 in culture from cell clones of second-round selection at 32° C.

FIG. 2B is a gel image of a western blot of cells from day 6 in culture from cell clones of second-round selection at 37° C.

FIG. 3 is a gel image of a western blot of cells from day 5 in culture for B1STBSTbSTh2cp13 and B1STBSTbSTh2cp15 cell clones at 32° C. and 37° C.

FIG. 4 is a representative image of southern blot analysis of the rCol7 from cell clone B1STBSTbcp03 (Master Cell Bank (MCB)), for confirmation of identity. Genomic DNA from rCol7 MCB (Lane 2) and non-transfected CHO cells (Lane 3) was digested with HindIII/XbaI enzymes and analyzed by Southern blotting using rCol7 coding sequence hybridization probe. Expected size of hybridizing band is 8.9 kb. Lane 1: HindIII size markers.

FIG. 5 is an image of a western blot analysis of reference collagen 7 (Lane 2) and MCB-derived rCol7 composition (Lane 3). Lane 1: molecular weight ladder.

FIG. 6 shows a plot of laminin-332 binding Kd versus the percentage fraction of Y1033 in the reference collagen 7 as well as several MCB-derived rCol7 compositions. Analysis indicates a lack of correlation of laminin 332 binding response to rCol7Y1033 content.

FIG. 7 is wound healing assay using keratinocytes with reference collagen 7 and MCB derived rCol7 compositions. A BSA control sample is also included in the analysis.

FIG. 8 demonstrates an intact transgene transcript present in both clones B1STBSTbSThcp13-cp01 (13-01) and B1STBSTbSThcp13-cp03 (13-03). The transcript was blotted using probes specific to NC1 and TH3 region of collagen 7.

FIG. 9 depicts the intracellular staining for Collagen 7 in clones B1STBSTbSThcp13-cp01 and B1STBSTbSThcp13-cp03.

FIG. 10 demonstrates HCP quantification for cell clones B1STBSTbSThcp13-cp01 and B1STBSTbSThcp13-cp03.

FIG. 11 demonstrates the rCol7 composition identity by SDS-PAGE from cell clones B1STBSTbSThcp13-cp01 (lane 8) and B1STBSTbSThcp13-cp03 (lane 7). Lane 1 is MW standard and lanes 2-6 are assay controls.

### DETAILED DESCRIPTION OF THE DISCLOSURE

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure relates to production systems, engineered host cells and methods for producing collagen 7 compositions. The collagen 7 composition may comprise human rCol7 and/or functional variants thereof. Preferably, the production systems comprise genetically engineered host cells for expressing human recombinant collagen 7 (rCol7) and/or functional variants thereof. The host cells may be genetically modified to comprise polynucleotides that encode recombinant collagen 7 and/or functional variants thereof. The host cells may be transformed with one or more expression vectors comprising polynucleotides encoding recombinant collagen 7 or functional variants thereof. In accordance with the present disclosure, the host cells are further transformed to express one or more protein that can increase rCol7 expression in the host cells. Such proteins may include prolyl 4-hydroxylase, prolidase, chaperone proteins and/or heat shock proteins, e.g., HSP 47.

The present disclosure provides collagen 7 compositions comprising rCol7 (e.g., human rCol7) and functional variants thereof, which may be produced by the production systems and host cells of the present disclosure. Further, pharmaceutical compositions and/or formulations comprising the collagen 7 compositions of the disclosure, are provided.

The present disclosure further provides vectors and methods for generating cell expression systems for producing collagen 7 compositions. In another aspect of the present disclosure, methods for inhibiting, alleviating or preventing the progression of a skin condition in a subject in need, with pharmaceutical compositions and/or formulations comprising rCol7 and functional variants thereof, are provided.

### Definitions

For convenience, certain terms employed in the specification, examples, and claims are collected here. Unless defined otherwise, all technical and scientific terms used herein have the same meaning as commonly understood by one ordinary skilled in the pertinent art. The following terms and phrases are intended to have the meanings as defined here.

As used herein, “collagen 7” (also referred to as C7, Col7, type VII collagen, and Collagen VII) refers to a collagen protein composed of three identical alpha chain polypeptides which are encoded by the COL7A1 gene. Each alpha chain polypeptide consists of 2,944 amino acids, including a central collagenous triple-helical segment (TH) (residues 1254-2783 in the mature peptide), flanked by a large global amino-terminal non-collagenous NC1 domain (residues 17-1253) and a smaller carboxyl-terminal non-collagenous NC2 domain (residues 2784-2944). The full-length alpha chain polypeptide of human collagen 7 comprises the amino acid sequence of SEQ ID NO.: 1 (Ref. NO.: NP_000085), which is encoded by the nucleic acid sequence of SEQ ID NO.: 2 (Ref. NO.: NM_000094).

Collagen 7 is the main constituent of anchoring fibrils, which serve as attachment complexes at the interface between the epithelial and mesenchymal layers of several tissues including the skin, oral mucosa, and cervix (Chung et al., Dermatol. Clin., 2010, 28(1):93-105). The anchoring fibrils in the skin are located below the basal lamina at the dermal-epidermal basement membrane zone (BMZ), securing the association between the epidermal BMZ and the dermis and contributing to the integrity of the skin (Varki et al. J Med Genet 2007, 44:181-192). The collagen 7 proteins form a nonstaggered array of disulfide bond stabilized dimeric aggregates (Burgenson et al, Ann N Y Acad Sci., 1985, 460:47-57). It should be noted that the term “collagen 7” as used herein, encompasses functional variants thereof, even in the absence of explicit recitation.

As used herein, the term “polypeptide” refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. A “protein” as used herein refers to one or more polypeptides that function as a discrete unit. In some contexts, the terms “polypeptide” and “protein” may be used interchangeably. The term “amino acid” as used herein refers to any of the twenty naturally occurring amino acids that are normally used in the formation of proteins and polypeptides, or analogs or derivatives of those amino acids. A “recombinant protein” or “recombinant polypeptide” refers to a protein or polypeptide molecule expressed utilizing isolated nucleic acid molecules or recombinant nucleic acid molecules. An “isolated” protein or nucleic acid molecule refers to a protein that is removed from its natural environment. Isolated proteins or nucleic acid molecules can be “of at least” a certain degree of purity if the protein or nucleic acid molecule of interest is at least 5%, 10%, 25%, 30%, 40%, 50%, 60%, 65%, 70%, 75%, 80%, 85%, 90%, 91%, 92%, 93%, 94%, 95%, 96%, 97%, 98%, or 99% pure on a weight-by-weight basis.

As used herein, the term “variant” or “functional variant” means any derivative of a wild type collagen 7 protein that essentially maintains the biological functions or activities of wild-type collagen 7.

Functional variants of collagen 7 may include polypeptides that maintain collagen 7's biological functions, such as the ability to form anchoring fibrils between the epidermal and dermal layers of human skin. A collagen 7 variant may have substantial identity with wild type collagen 7. Collagen 7 variants include, but are not limited to, collagen 7 polypeptides that have been either chemically modified relative to wild-type collagen 7 and/or contain one or more amino acid sequence alterations relative to wild-type collagen 7. In some embodiments, variants of collagen 7 may include polypeptides having at least 70%, 75%, 80%, 85%, 90%, 91%, 92%, 93%, 94%, 95%, 96%, 97%, 98% or 99% identity with the amino acid sequence of human collagen 7 (wild-type). As a non-limiting example, a collagen 7 variant may comprise a polypeptide having at least 70%, 75%, 80%, 85%, 90%, 91%, 92%, 93%, 94%, 95%, 96%, 97%, 98% or 99% identity with the amino acid sequence of SEQ ID NO.: 1.

Variants of collagen 7 protein may also include polypeptides having amino acid modifications (e.g., deletions, additions or substitutions, such as conservative substitutions) from the amino acid sequence of wild type collagen 7 (e.g., SEQ ID NO.: 1), and/or other chemical modifications of the amino acid residues. In some embodiments, a variant of collagen 7 differs by about 1-50 amino acid residues, or about 1-30 amino acid residues, or about 1-20 amino acid residues, or about 1-10 amino acid residues from human collagen 7. A variant of collagen 7 may differ by 1, 2, 3, 4, 5, 6, 7, 8, 9, 10, 15, 20, 25, 30, 35, 40, 45 or 50 amino acid residues from human collagen 7. As a non-limiting example, the variant may differ by 1, 2, 3, 4, 5, 6, 7, 8, 9, 10, 15, 20, 25, 30, 35, 40, 45, or 50 amino acid residues from the amino acid sequence of SEQ ID NO.: 1. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Chemical modifications of amino acid residues include, but are not limited to, glycosylation, phosphorylation, amidation, myristoylation, hydroxylation, phosphopantetheine attachment, methylation, and prenylation.

In accordance with the present disclosure, a functional variant of collagen 7 may also include a “functional fragment” of collagen 7, which refers to a portion of human collagen 7 polypeptide that is a shorter polypeptide than the full-length protein but maintains its biological function, such as the ability to form anchoring fibrils between the epidermal and dermal layers of human skin and the ability to bind collagen 4 and laminin-332. A functional fragment of human collagen 7 may not include the entirety of collagen 7's 2,944 amino acid residues. For example, a functional fragment may include all or a portion of the NC1 domain and/or the NC2 domain of collagen 7, e.g., the functional fragment can be collagen 7 without all or a portion of the central collagenous helical domain.

As used herein, the term “collagen 7 composition” refers to a composition comprising a plurality of recombinant collagen 7 alpha polypeptides, a plurality of collagen 7 equivalent polypeptides, or a plurality of functional variants and fragments thereof. Alternatively, a collagen 7 composition may comprise a mixture of a plurality of recombinant collagen 7 alpha polypeptides, a plurality of collagen 7 equivalent polypeptides, and a plurality of functional variants and fragments thereof. In some examples, a collagen 7 composition comprises collagen 7 alpha polypeptide having an amino acid sequence of SEQ ID NO.: 1. The collagen 7 composition may be produced by the present expression system and host cells that are engineered to express rCol7 and/or functional variants thereof. The collagen 7 composition may be purified from the culture media of the host cells.

As used herein, the terms “polynucleotide” and “nucleic acid molecule” are used interchangeably and refer to a polymer of nucleotides joined together by a phosphodiester linkage between 5′ and 3′ carbon atoms. A polynucleotide can include, but is not limited to, RNA (ribonucleic acid molecule) (e.g., mRNA) and DNA (deoxyribonucleic acid molecule) (e.g., cDNA from eukaryotic mRNA, genomic DNA sequences from eukaryotic (e.g., mammalian) DNA, and even synthetic DNA sequences). The term also captures sequences that include any of the known base analogs of DNA or RNA. The term “recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. In accordance with the present disclosure, polynucleotides that encode collagen 7 may comprise the nucleic acid sequence of SEQ ID NO.: 2. The sequences may be codon optimized nucleic acid sequences, and in one embodiment, this codon optimization may serve to increase glycine content.

The term “substantially identical” refers to a nucleic acid or amino acid sequence that, when optimally aligned, for example using the methods described below, shares at least 60%, 65%, 70%, 75%, 80%, 85%, 90%, 91%, 92%, 93%, 94%, 95%, 96%, 97%, 98%, 99%, or 100% sequence identity with a second nucleic acid or amino acid sequence. “Substantial identity” may be used to refer to various types and lengths of sequence, such as full-length sequences, functional domains, coding and/or regulatory sequences, exons, introns, promoters, and genomic sequences. Percent sequence identity between two polypeptides or nucleic acid sequences is determined in various ways that are within the skill in the art, for instance, using publicly available computer software such as BLAST (Basic Local Alignment Search Tool; (Altschul, S. F., W. Gish, et al. J Mol Biol., 1990, 215:403-10), BLAST-2, BLAST-P, BLAST-N, BLAST-X, WU-BLAST-2, ALIGN, ALIGN-2, CLUSTAL, or Megalign (DNASTAR). In addition, those skilled in the art can determine appropriate parameters for measuring alignment, including any algorithms needed to achieve maximal alignment over the length of the sequences being compared. It is understood that for the purposes of determining sequence identity when comparing a DNA sequence to an RNA sequence, a thymine nucleotide is equivalent to an uracil nucleotide. Conservative substitutions typically include substitutions within one of the following groups: glycine, alanine; valine, isoleucine, leucine; aspartic acid, glutamic acid, asparagine, glutamine; serine, threonine; lysine, arginine; and phenylalanine, tyrosine.

The polynucleotide may include from about 30 to about 200,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, from 2,000 to 100,000, from 5,000 to 15,000, and from 5,000 to 20,000 nucleotides).

The polynucleotide encoding a polypeptide may be chemically modified. As used herein, the term “modification” may include any chemical modifications of the nucleoside base. The polynucleotide may comprise at least one chemically modified nucleoside, a cytidine modification, a guanosine modification, and/or a thymidine modification. The polynucleotide may comprise 2, 3, 4, 5, 6, 7, 8, 9, 10, or more chemically modified nucleosides.

As used herein, the term “vector” means viral or non-viral, prokaryotic or eukaryotic, deoxyribonucleic acid, ribonucleic acid or a nucleic acid analog, that is capable of carrying another nucleic acid molecule, e.g., a polynucleotide encoding recombinant collagen 7 or a functional variant thereof. A vector may carry a nucleic acid molecule into a cell, referred to as “host cell”, so that all or a part of the nucleic acid molecule is transcribed or expressed. Vectors are frequently assembled as composites of elements derived from different viral, bacterial, or mammalian genes. Vectors contain various coding and non-coding sequences including sequences coding for selectable markers (e.g., an antibiotic resistance gene), sequences that facilitate their propagation in bacteria, or one or more transcription units that are expressed only in certain cell types. For example, mammalian expression vectors often contain both prokaryotic sequences that facilitate the propagation of the vector in bacteria and one or more eukaryotic transcription units that are expressed only in eukaryotic cells. It will be appreciated by those skilled in the art that the design of the expression vector can depend on such factors as the choice of the host cell to be transformed, the level of expression of protein desired, etc. Suitable vectors for use herein may also contain a selectable marker gene that encodes a product necessary for the host cell to grow and survive under specific conditions, aiding in the selection of host cells into which the vector has been introduced. Typical selection genes may include, but are not limited to, genes encoding a protein that confers resistance to an antibiotic, drug, or toxin (e.g., tetracycline, ampicillin, neomycin, hygromycin, etc.). An expression vector may be a bacterial plasmid, bacteriophage, yeast plasmid, plant virus or mammalian cell virus, such as adenovirus, retrovirus or any other vehicles known in the art. Suitable vectors include, for example, plasmids, phagemids, cosmids and viral vectors.

Standard methods, known to those skilled in the art, may be used to construct the recombinant expression vectors containing the nucleic acid sequences described herein. These methods include, but are not limited to, in vitro recombinant techniques, synthetic techniques, and in vivo recombination/genetic recombination. The choice of method depends on the nature of the specific nucleotide fragments and may be determined by persons skilled in the art.

As used herein, the terms “transformed” and “transfected” encompass the introduction of a nucleic acid (e.g. a vector) into a cell by a number of techniques known in the art. Transformation and transfection techniques include, but are not limited to, calcium phosphate or calcium chloride coprecipitation, DEAE-dextran-mediated transfection, lipofectamine, electroporation, microinjection, and viral mediated transfection. A person skilled in the art would have knowledge of suitable transformation and transfection methods based on the host cell/vector combination. For long term, high yield production of recombinant proteins, stable expression of the recombinant protein may be preferred. Host cells that stably express the recombinant protein may be engineered. In some examples, the term “supertransfection” is used to referred to cell transfection with multiple exogenous polynucleotides and vectors, such as 2, 3, 4, 5 or more exogenous polynucleotides and vectors.

As used herein, the term “host cell” refers to any living cell capable of expressing an exogenous protein, such as the protein encoded by an expression vector. Host cells may be prokaryotic or eukaryotic cells into which a recombinant expression vector can be introduced. The term “host cell” refers not only to the particular subject cell but to the progeny or potential progeny of the particular subject cell. Because certain modifications may occur in succeeding generations due to either mutation or environmental influences, such progeny may not in fact, be identical to the parent cell, but are still included within the scope of the term as used herein. Exemplary host cells may be derived from yeast, fungi, insect or mammalian systems, but are not limited to this selection. Suitable host cells may include primary or transformed cell lines, including, but not limited to, fibroblasts, keratinocytes, CHO, HEK293, C127, VERO, BHK, HeLa, COS, MDCK, etc. Other suitable host cells are known to those skilled in the art. The host cell may be able to modulate expression of transformed nucleic acid sequences including the coding sequences included in the vector, and to modify and process the gene product encoded in the vector sequence in a specific manner. Modifications, including, but not limited to, glycosylation, phosphorylation and processing of protein products may be important to the function of a protein.

As used herein, the term “production system” or “expression system” refers to a system that can produce a polypeptide or protein of interest. The production system may comprise cells that can express the polypeptide or protein of interest, e.g., collagen 7. In the context of the present disclosure, the production system comprises host cells that are engineered to express rCol7 and/or functional variants thereof. The production system may further comprise a reactor suitable for the growth of the host cells engineered to express rCol7 and/or functional variants thereof. The production reactor as used herein refers to the final bioreactor used in the production of the polypeptide or protein of interest, i.e. rCol7. The reactor may be any size. As non-limiting examples, the reactor may be at least 500 mL, at least 1 liter and may be, 2, 3, 4, 5, 6, 7, 8, 9, 10, 20, 30, 40, 50, 60, 70, 80, 90, 100, 200, 250, 300, 350, 400, 450, 500, 600, 700, 800, 900, 1,000, 2,500, 3,000, 4,000, 5,000, 6,000, 7,000, 8,000, 9,000, 10,000 liters or more, or any volume in between. The volume of the large-scale cell culture production reactor is typically at least 20 liters, or at least 50 liters, or at least 100 liters, or at least 200 liters, or at least 300 liters, or at least 400 liters, or at least 500 liters, or may be 1000, 2500, 3,000, 4,000, 5000, 6,000, 7,000, 8,000, 10,000, 12,000 liters or more, or any volume in between. The reactor can be composed of any material that is suitable for holding cell cultures suspended in media under the culture conditions of the present disclosure, including glass, plastic or metal. The conditions of the production reactor are typically controlled during the cell culturing period to ensure cell density and viability. These conditions include, but are not limited to, pH, temperature, humidity and CO2 supply. As used herein, the term “cell density” refers to the number of cells present in a given volume of medium. The term “cell viability” as used herein refers to the ability of cells in culture to survive under a given set of culture conditions or experimental variations. The term as used herein also refers to the portion of cells which are alive at a particular time in relation to the total number of cells (living and dead) in the culture at that time.

As used herein, the term “bioreactor” is used to refer to the reactor for culturing host cells to express collagen 7 compositions. A bioreactor may be a traditional non-disposable reactor, or a disposable bioreactor.

As used herein, a “patient” or a “subject” to be treated may mean either a human or a non-human mammal. In the context of the disclosure, the term “patient” or “subject” refers to any subject, preferably a mammal, and more preferably a human, afflicted with a skin disorder, such as epidermolysis bullosa (e.g., dystrophic epidermolysis bullosa).

As used herein, the term “disease” or “disorder” refers to a pathological condition of a part, organ, or system of an organism resulting from various causes, such as autoimmune defect, genetic defect or environmental stress, and characterized by an identifiable group of signs or symptoms. A “skin disease” or “skin disorder” means a clinical condition of the skin, such as a condition that affects the skin in a subject, for example, a bullous disorder, an inflammatory skin condition, or a skin cancer. Bullous (blistering) disorders are a group of heterogeneous disorders characterized by elevated fluid-filled blistering lesions (bullae) that primarily are on the skin and mucous membranes. Bullae can be variable in sizes and the specific symptoms and severity of blistering diseases vary from one person to another, even among individuals with the same disorder. Exemplary blistering disorders include, but are not limited to, epidermolysis bullosa acquisita (EBA) and congenital epidermolysis bullosa (EB) such as dystrophic EB. EB includes a group of inherited connective tissue diseases that cause blisters on the skin and mucous membranes resulting from genetic defects. Dystrophic epidermolysis bullosa (DEB) is mostly caused by mutations within the COL7A1 gene, which encodes collagen 7 protein. To date, about 400 mutations in COL7A1 have been reported (van den Akker et al., Hum Mutat. 2011, 32(10):1100-1107). DEB has two patterns of inheritance: autosomal dominant (DDEB) and autosomal recessive (RDEB). DDEB involves reduced collagen 7 expression which is generally caused by glycine substitutions within the collagenous domain of the collagen alpha₁ (VII) chain. RDEB is usually severe and caused by absence or marked reduction of collagen 7 expression, mostly due to premature termination codons (PTC) in the COL7A1 gene.

As used herein, “treating” a patient or “treatment”, or “to treat” refers to administering to the subject a pharmaceutical composition, such that at least one symptom of a disease is reversed, cured, alleviated or decreased. “Treating” EB, e.g., DEB (DDEB and RREB), in a subject or treatment of EB, or to treat EB refers to administering to the subject having an EB a pharmaceutical composition, e.g., a collagen 7 composition comprising rCol7 and functional variants thereof, such that at least one symptom of the EB disease is reversed, cured, alleviated or decreased. The symptoms of an EB disease may that may be targeted for treatment include, but are not limited to, blistering; lesions (e.g., rectal, anal, urethral lesions and/or mucosal lesions and/or lesions of squamous epithelial tissue); lesions of the gastrointestinal tract; contractures, e.g., flexion contractures (e.g., of the extremities); pseudosyndactyly of the hands or feet; carcinoma (e.g., squamous cell carcinoma); bulla formation; nail and/or teeth deformities; constricted esophagus; eye disorders, anemia, malnutrition; secondary skin infection; sepsis; hoarse voice; urethral stenosis; phimosis; corneal scarring; malabsorption; and failure to thrive.

As used herein, the term “preventing” or “prevent”, or “prevention” means the administration of a composition, e.g., prior to clinical manifestation of the unwanted condition (e.g., disease or other unwanted state of the host animal) so that it protects the host against developing the unwanted condition, e.g., at least one symptom of the disease is prevented. “Preventing” a disease may also be referred to as “prophylaxis” or “prophylactic treatment.” In the context of the present disclosure, one or more symptoms associated with EB, e.g., scarring can be prevented. Scarring in a subject with EB may result in one or more of the following symptoms: contractures, e.g., flexion contractures (e.g., of the extremities); pseudosyndactyly of the hands or feet; carcinoma (e.g., squamous cell carcinoma); rectal lesions; mucosal lesions; bulla formation; bulla formation post manual trauma; nail or teeth deformities; constricted esophagus; eye disorders, anemia, malnutrition; secondary skin infection; sepsis; hoarse voice; urethral stenosis; phimosis; corneal scarring; malabsorption; and failure to thrive.

As used herein, the term “therapeutically effective amount” refers to an amount effective, at dosages and for periods of time necessary, to achieve the desired therapeutic outcome. A therapeutically effective amount of a composition may vary depending on factors such as the disease state or age, sex, and weight of a subject. A therapeutically effective amount is also one in which any toxic or detrimental effects of the composition are outweighed by the therapeutically beneficial effects. In the context of the present disclosure, an effective amount of rCol7, when administered as part of any defined treatment regimen, produces a statistically measurable improvement in outcome, as evidenced by at least one clinical parameter associated with the complication.

The compositions of the present disclosure may be administered in combination with another agent or therapy. As used herein, the term “combination” refers to the use of two or more agents or therapies to treat the same patient, wherein the use or action of the agents or therapies overlap in time. In the context of the present disclosure, a pharmaceutical composition comprising rCol7 or a functional variant thereof may be used in combination with one or more agents that prevent and treat a skin disorder, such as DEB. The agents or therapies can be administered at the same time (e.g., as a single formulation that is administered to a patient or as two separate formulations administered concurrently) or sequentially in any order. In some embodiments, the delivery of one agent or therapy is still occurring when the delivery of the second begins, so that there is overlap in terms of administration. This is sometimes referred to herein as “simultaneous” or “concurrent delivery”. In other embodiments, the delivery of one agent or therapy ends before the delivery of the other begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

As used herein, the term “pharmaceutically acceptable” refers to being compatible with the other ingredients of the formulation and not deleterious to the recipient thereof.

### Production Systems and Cells for Producing Collagen 7 Compositions

In one aspect of the present disclosure, a production system for preparing a collagen 7 composition comprising human rCol7, and/or functional variants thereof is provided, wherein the production system comprises host cells for expressing human rCol7, and/or functional variants thereof. The host cells are genetically modified to express rCol7, and in some embodiments, are further genetically engineered to express at least one protein that can increase the rCol7 expression in the host cells. The protein may include, but is not limited to, a prolidase (also known as peptidase D, proline dipeptidase, and L-proline hydrolase, PEPD) or a functional variant thereof, a prolyl 4-hydroxylase (also known as procollagen-proline, and 2-oxoglutarate 4-dioxygenase, P4H) or a functional variant thereof, a C1GALT1 Specific chaperone 1 (also known as core 1 beta3-galactosyltransferase-specific molecular chaperone, Beta 1,3-galactosyltransferase 2, and COSMC) or a functional variant thereof, lysyl hydroxylase (LH) or a functional variant thereof, a glycosyl-transferase (GTF) or a functional variant thereof, and a heat shock protein or a functional variant thereof (e.g., HSP 47). Preferably, the protein used to increase the expression of rCol7 in the host cells is human prolyl 4-hydroxylase (hP4H), comprising an alpha polypeptide (subunit A) of human prolyl 4-hydroxylase (e.g. hP4HA1) or a functional variant thereof, and a beta polypeptide (subunit B) of human prolyl 4-hydroxylase (i.e. hP4HB) or a functional variant thereof, and HSP 47 or a functional variant thereof.

In some embodiments, the host cells of the present production system are transformed or transfected to express a rCol7 alpha chain polypeptide, and at least one protein that can increase rCol7 expression in the host cells, selecting from prolidase (PEPD), prolyl 4-hydroxylase (P4H), lysyl hydroxylase (LH), a glycosyl-transferase (GTF), C1GALT1 Specific chaperone 1 (COSMC), or head shock protein HSP 47, or a functional variant thereof. Preferably the host cells are transformed with human P4H comprising an alpha polypeptide (subunit A) of human prolyl 4-hydroxylase (e.g. hP4HA1) and a beta polypeptide (subunit B) of human prolyl 4-hydroxylase (i.e. hP4HB) and/or HSP 47.

In some embodiments, the host cells of the present production system are genetically engineered to produce human rCol7, and/or functional variants thereof. The human rCol7 may comprise an amino acid sequence presented by SEQ ID NO.: 1. The rCol7 may also have an amino acid sequence at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to the amino acid sequence of SEQ ID NO.: 1. A functional variant of human collagen 7 may have at least 1, 2, 3, 4, 5, 6, 7, 8, 9, 10, 11, 12, 13, 14, 15, 16, 17, 18, 19, 20, or more amino acid differences to the sequence shown as SEQ ID NO.: 1. The functional variant of human collagen 7 may comprise a fragment having a portion of the amino acid sequence of SEQ ID NO.: 1.

In some embodiments, the rCol7 may be encoded by a polynucleotide comprising a nucleic acid sequence presented by SEQ ID NO.: 2. In some embodiments, the polynucleotide encoding rCol7 or a functional variant thereof may comprise a nucleic acid sequence at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to the nucleic acid sequence of SEQ ID NO.: 2.

In some embodiments, the host cells of the present production system may be genetically engineered to further express prolidase (PEPD). Prolidase is a cytosolic imidodipeptidase encoded by the PEPD gene that can hydrolyze dipeptides or tripeptides with C-terminal proline or hydroxyproline residues. This enzyme plays an important role in the recycling of proline from imidodipeptides, mostly derived from degradation products of collagen, for resynthesis of collagen and other proline containing proteins. This enzyme can facilitate the synthesis of the recombinant collagen protein in some host cells 23n some embodiments, the host cells of the present production system may be modified to express a prolidase or a functional variant thereof. The prolidase may be a mammalian prolidase, or a functional variant thereof, e.g., a human prolidase, a mouse prolidase, a rat prolidase, or a hamster prolidase. As a non-limiting example, the prolidase is a human prolidase comprising an amino acid sequence of SEQ ID NO.: 3. A functional variant of a prolidase may have an amino acid sequence that is at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to the amino acid sequence of SEQ ID NO.: 3.

In some embodiments, the prolidase may be encoded by a polynucleotide comprising a nucleic acid sequence presented by SEQ ID NO.: 4. The polynucleotide encoding prolidase or a functional variant thereof, may comprise a nucleic acid sequence at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to the nucleic acid sequence of SEQ ID NO.: 4.

In some embodiments, the host cells of the present production system may be genetically engineered to further express prolyl 4-hydroxylase (P4H). Prolyl 4-hydroxylase is an enzyme involved in hydroxylation of prolyl residues in preprocollagen, an important step for processing procollagen to mature collagen protein. In some embodiments, the host cells of the present production system may be modified to express prolyl 4-hydroxylase or a functional variant thereof. The prolyl 4-hydroxylase may be a mammalian prolyl 4-hydroxylase or a functional variant thereof, e.g., a human prolyl 4-hydroxylase, a mouse prolyl 4-hydroxylase, a rat prolyl 4-hydroxylase, or a hamster prolyl 4-hydroxylase. Mammalian prolyl 4-hydroxylase is an α2β2 tetramer, composed of two identical alpha (α) polypeptides (subunit A) and two beta (β) polypeptides (subunit B). The alpha polypeptide (P4Ha or P4HA) contains the peptide-substrate-binding domain and the enzymic active site. The alpha polypeptide may be an alpha polypeptide I (alpha-1, P4Ha (I), subunit A1 or P4HA1) or an isoform thereof, alpha polypeptide II (alpha-2, P4Ha (II), subunit A2, or P4HA2) or an isoform thereof, or alpha polypeptide III (alpha-3, P4Ha (III), subunit A3, or P4HA3) or an isoform thereof.

In some embodiments, the host cells of the present production system are engineered to express a human prolyl 4-hydroxylase comprising two alpha polypeptides (subunit A) and two beta polypeptides (subunit B) or functional variants thereof. The alpha polypeptide of human prolyl 4-hydroxylase may be an alpha polypeptide 1 an alpha polypeptide 2 or an alpha polypeptide 3 or an isoform or functional variants thereof. The alpha polypeptide 1 (alpha-1) may comprise an amino acid sequence selected from the group consisting of SEQ ID NOs.: 5, 7 and 9. The alpha polypeptide 2 (alpha-2) may comprise an amino acid sequence selected from the group consisting of SEQ ID NOs.: 11 and 13. The alpha polypeptide 3 (alpha-3) may comprise an amino acid sequence selected from the group consisting of SEQ ID NOs.: 15 and 17. The beta polypeptide may comprise an amino acid sequence of SEQ ID NO.: 19. In some embodiments, the alpha polypeptide 1 may be encoded by a polynucleotide that comprises a nucleic acid sequence selected from the group consisting of SEQ ID NOs.: 6, 8 and 10. The alpha polypeptide 2 may be encoded by a polynucleotide that comprises a nucleic acid sequence selected from the group consisting of SEQ ID NOs.: 12 and 14. The alpha polypeptide 3 may be encoded by a polynucleotide that comprises a nucleic acid sequence selected from the group consisting of SEQ ID NOs.: 16 and 18. The beta polypeptide may be encoded by a polynucleotide that comprises a nucleic acid sequence presented by SEQ ID NO.: 20.

A functional variant of alpha-1 polypeptide (subunit A1) of prolyl 4-hydroxylase may have an amino acid sequence that is at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to any of the amino acid sequences given as SEQ ID NOs.: 5, 7 and 9. A functional variant of beta polypeptide (subunit B) of prolyl 4-hydroxylase may have an amino acid sequence that is at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to the amino acid sequence of SEQ ID NO.: 19.

As a non-limiting example, the host cells of the present production system are engineered to express human prolyl 4-hydroxylase consisting of two alpha-1 polypeptides (subunit A1), each comprising an amino acid sequence selected from the group consisting of SEQ ID NOs.: 5, 7 and 9, and two beta polypeptides (subunit B), each comprising an amino acid sequence of SEQ ID NO.: 19.

In some embodiments, the alpha-1 polypeptide (subunit A1) of prolyl 4-hydroxylase may be encoded by a polynucleotide comprising a nucleic acid sequence selected from the group consisting of SEQ ID NOs.: 6, 8 and 10. In some embodiments, the polynucleotide encoding the alpha-1 polypeptide or a functional variant thereof, may comprise a nucleic acid sequence at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to any of the nucleic acid sequences given as SEQ ID NOs.: 6, 8 or 10. In some embodiments, the beta polypeptide (subunit B) of prolyl 4-hydroxylase may be encoded by a polynucleotide comprising a nucleic acid sequence presented by SEQ ID NO.: 20. In some embodiments, the polynucleotide encoding the beta polypeptide or a functional variant thereof, may comprise a nucleic acid sequence at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to the nucleic acid sequence of SEQ ID NO.: 20.

C1GALT1 specific chaperone 1 (COMSC) acts as molecular chaperone for collagen folding, stability and activity. In some embodiments, the host cells of the present production system may be modified to express a C1GALT1 specific chaperone 1 or a functional variant thereof. The C1GALT1 specific chaperone 1 may be of mammalian origin, such as, but not limited to, human, mouse, rat, or a hamster. As a non-limiting example, the host cells are modified to express a human C1GALT1 specific chaperone 1 comprising an amino acid sequence of SEQ ID NO.: 21. A functional variant of C1GALT1 specific chaperone 1 may have an amino acid sequence that is at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to the amino acid sequence of SEQ ID NO.: 21.

In some embodiments, the C1GALT1 specific chaperone 1 polypeptide may be encoded by a polynucleotide comprising a nucleic acid sequence presented by SEQ ID NO.: 22. In some embodiments, the polynucleotide encoding the C1GALT1 specific chaperone 1 polypeptide or a functional variant thereof, may comprise a nucleic acid sequence at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to the nucleic acid sequence of SEQ ID NO.: 22.

Heat shock protein 47 (aka Serpin H1, and colligin) is a unique collagen-specific molecular chaperone (reviewed by Nagata et al., Trends Biochem Sci., 1996, 21:22-26) which binds specifically to collagenous peptides for facilitating collagen protein folding, assembly and intracellular transport. In some embodiments, the host cells of the present production system may be modified to express HSP 47 or a functional variant thereof. HSP 47 may be of mammalian origin, such as, but not limited to, human, mouse, rat, or a hamster. As a non-limiting example, the host cells are modified to express a human HSP 47 comprising an amino acid sequence of SEQ ID NO.: 23. A functional variant of HSP 47 may have an amino acid sequence that is at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to the amino acid sequence of SEQ ID NO.: 23.

In some embodiments, the HSP 47 polypeptide may be encoded by a polynucleotide comprising a nucleic acid sequence presented by SEQ ID NO.: 24. In some embodiments, the polynucleotide encoding HSP 47 or a functional variant thereof, may comprise a nucleic acid sequence at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, identical to the nucleic acid sequence of SEQ ID NO.: 24. The aforementioned sequences are summarized below, in Table 1.

TABLE 1
Reference Sequences

| | Protein | SEQ | Polynucleotide | SEQ |
|---|---|---|---|---|
| Polypeptide | Ref. NO.: | ID NO.: | Ref. NO.: | ID NO.: |
| Collagen 7 | NP_000085 | 1 | NM_000094 | 2 |
| Prolidase | NP_000276 | 3 | NM_000285 | 4 |
| Prolyl hydroxylase, alpha | NP_000908 | 5 | NM_000917 | 6 |
| polypeptide I (SubunitA1, | | | | |
| alpha-1), isoform 1 | | | | |
| Prolyl hydroxylase, alpha | NP_001017962 | 7 | NM_001017962 | 8 |
| polypeptide I (SubunitA1, | | | | |
| alpha-1), isoform 2 | | | | |
| Prolyl hydroxylase, alpha | NP_001136068 | 9 | NM_001142596 | 10 |
| polypeptide I (SubunitA1, | | | | |
| alpha-1), isoform 3 | | | | |
| Prolyl hydroxylase, alpha | NP_001136071 | 11 | NM_001142599 | 12 |
| polypeptide II (Subunit A2, | | | | |
| alpha-2), isoform 1 | | | | |
| Prolyl hydroxylase, alpha | NP_001136070 | 13 | NM_001142598 | 14 |
| polypeptide II (Subunit A2, | | | | |
| alpha-2), isoform 2 | | | | |
| Prolyl hydroxylase, alpha | NP_878907 | 15 | NM_182904 | 16 |
| polypeptide III (Subunit A3, | | | | |
| alpha-3), isoform 1 | | | | |
| Prolyl hydroxylase, alpha | NP_001275677 | 17 | NM_001288748 | 18 |
| polypeptide III (Subunit A3, | | | | |
| alpha-3), isoform 2 | | | | |
| Prolyl 4-hydroxylase, beta | NP_000909 | 19 | NM_000918 | 20 |
| polypeptide (Subunit B) | | | | |
| Chaperone protein | NP_001011551 | 21 | NM_001011551 | 22 |
| (COMSC) | | | | |
| Heat shock protein 47 | NP_001226.2 | 23 | NM_001235.3 | 24 |
| (HSP47) | | | | |

In some embodiments, the host cells of the present production system are modified to express human rCol7, and the alpha and beta polypeptides of hP4H that can increase the expression of rCol7, wherein the alpha polypeptide (P4HA) may be an alpha polypeptide I, an alpha polypeptide II or an alpha polypeptide III or isoforms thereof.

In some embodiments, the host cells of the present production system are modified to express human rCol7, the alpha and beta polypeptides of hP4H, and human HSP 47.

In some embodiments, the host cells of the present production system are genetically modified to comprise at least one first exogenous polynucleotide encoding rCol7, and at least one exogenous polynucleotide encoding a protein that can increase the expression of rCol7 in the host cells, wherein the protein may include, but is not limited to, prolidase, prolyl 4-hydroxylase comprising an alpha polypeptide (subunit A) and a beta polypeptide (subunit B), lysyl hydroxylase, glycosyl transferase, C1GALT1 Specific chaperone 1, a head shock protein, e.g., HSP 47, and functional variants thereof. Optionally, the host cells may be further modified to comprise a second exogenous polynucleotide encoding rCol7, or a functional variant thereof.

In some embodiments, the exogenous polynucleotide encoding rCol7 or a functional variant thereof, may comprise at least one modification such as codon optimization. In some embodiments, the exogenous polynucleotide encoding rCol7 may comprise optimized glycine codons in the sequence.

In one preferred embodiment, the host cells of the production system are genetically engineered to comprise at least one first exogenous polynucleotide that encodes human rCol7, an exogenous polynucleotide encoding an alpha polypeptide of human prolyl 4-hydroxylase (subunit A), and an exogenous polynucleotide encoding a beta polypeptide of human prolyl 4-hydroxylase (subunit B). The alpha polypeptide may be an alpha polypeptide I (alpha-1/subunit A1) or an isoform thereof, or an alpha polypeptide II (alpha-2/subunit A2) or an isoform thereof, an alpha polypeptide III (alpha-3/subunit A3) or an isoform thereof. In one preferred example, the alpha polypeptide is alpha polypeptide I (alpha-1/subunit A1) or an isoform or a functional variant thereof.

In some embodiments, the host cells of the production system are genetically engineered to comprise at least one first exogenous polynucleotide encoding human rCol7, an exogenous polynucleotide encoding an alpha polypeptide of human prolyl 4-hydroxylase (subunit A), and an exogenous polynucleotide encoding a beta polypeptide of human prolyl 4-hydroxylase (subunit B), and an exogenous polynucleotide encoding HSP 47.

Optionally, the host cells may further comprise a second exogenous polynucleotide encoding rCol7, or a functional variant thereof. In some examples, the first polynucleotide encoding rCol7 and the second polynucleotide encoding rCol7 may have the same nucleic acid sequence. In other examples, the two polynucleotides encoding rCol7 may have different nucleic acid sequences.

In some embodiments, the host cells of the present production system are genetically engineered to comprise a first exogenous polynucleotide encoding human rCol7 and having a nucleic acid sequence of SEQ ID NO.: 25, an exogenous polynucleotide encoding the alpha polypeptide 1 of human prolyl 4-hydroxylase having a nucleic acid sequence of SEQ ID NO.: 28, and an exogenous polynucleotide encoding the beta polypeptide of prolyl 4-hydroxylase having a nucleic acid sequence of SEQ ID NO.: 30. In one preferred embodiment, the first exogenous polynucleotide for expressing rCol7 comprises the nucleic acid sequence of SEQ ID NO.: 26; the exogenous polynucleotide encoding the alpha polypeptide 1 of prolyl 4-hydroxylase comprises a nucleic acid sequence of SEQ ID. NO.: 29; and the exogenous polynucleotide encoding the beta polypeptide of prolyl 4-hydroxylase comprises a nucleic acid sequence of SEQ ID NO.: 31.

In some embodiments, the host cells of the production system are further modified with a second exogenous polynucleotide encoding human rCol7 and having a nucleic acid sequence of SEQ ID NO.: 25. The second exogenous polynucleotide for expressing rCol7 may comprise the same nucleic acid sequence of, or a different nucleic acid sequence from, the first exogenous polynucleotide for expressing rCol7 or the functional variant thereof. In one example, the second exogenous polynucleotide for expressing rCol7 comprises the nucleic acid sequence of SEQ ID NO.: 27.

In some embodiments, the host cells are modified with two polynucleotides encoding rCol7, wherein the two polynucleotides have the nucleic acid sequences of SEQ ID NO.: 26 and SEQ ID NO.: 27.

In some embodiments, the host cells are further genetically engineered to comprise an exogenous polynucleotide encoding human HSP 47, wherein the polynucleotide sequence comprises a nucleic acid sequence of SEQ ID NO.: 32.

The host cells of the present disclosure may further be genetically modified to comprise an exogenous polynucleotide for expressing prolidase, or an exogenous polynucleotide for expressing C1GALT1 specific chaperone 1.

In other embodiments, the host cells of the production system may be transfected with one or more vectors each comprising one or more polynucleotide sequences encoding rCol7 and/or functional variants thereof.

In some embodiments, the host cells of the production system are genetically modified to comprise an expression vector comprising a polynucleotide encoding rCol7, or a functional variant thereof, and at least one expression vector comprising a polynucleotide that encodes a protein that can increase the rCol7 expression in the host cells, such as prolidase, prolyl 4-hydroxylase, lysyl hydroxylase, glycosyl transferase, C1GALT1 Specific chaperone 1, a heat shock protein (e.g., HSP 47), or functional variants thereof.

In one preferred embodiment, the host cells are genetically engineered to comprise a first expression vector comprising a first polynucleotide encoding human rCol7, or a functional variant thereof, an expression vector comprising a polynucleotide encoding an alpha polypeptide (subunit A) of human prolyl 4-hydroxylase or a functional variant thereof, and an expression vector comprising a polynucleotide encoding a beta polypeptide (subunit B) of human prolyl 4-hydroxylase or a functional variant thereof. Optionally, the host cells are further modified to comprise a second expression vector comprising a second polynucleotide that encodes human rCol7, or a functional variant thereof. The two polynucleotides encoding human rCol7 or functional variants thereof may comprise the same coding nucleic acid sequences. Alternatively, the two polynucleotides encoding human rCol7 or functional variants thereof may comprise different coding nucleic acid sequences. The first and second rCol7 expression vectors may comprise different selection marker genes, for example, two different antibiotic resistance markers. The selection antibiotics may include, but are not limited to, kanamycin, spectinomycin, streptomycin, ampicillin, carbenicillin, bleomycin, erythromycin, polymyxin B, tetracyclin and chloramphenicol.

In other embodiments, the host cells of the present production system may be genetically modified to comprise an expression vector comprising a first polynucleotide encoding human rCol7, or a functional variant thereof; and an expression vector comprising a polynucleotide encoding an alpha polypeptide (subunit A) of hP4H and a polynucleotide encoding a beta polypeptide (subunit B) of hP4H, or functional variants thereof.

In some embodiments, the host cells of the present production system may be genetically modified to comprise an expression vector comprising a first polynucleotide encoding human rCol7, or a functional variant thereof; and an expression vector comprising a polynucleotide encoding an alpha polypeptide (subunit A) of hP4H, or a functional variant thereof, and a polynucleotide encoding a beta polypeptide (subunit B) of hP4H, or a functional variant thereof, and an expression vector comprising a polynucleotide encoding human HSP 47.

As a non-limiting example, the host cells of the present production system may be genetically engineered to comprise a first expression vector comprising a polynucleotide sequence encoding human rCol7 and having a nucleic acid sequence of SEQ ID NO.: 25, an expression vector comprising a polynucleotide sequence encoding an alpha polypeptide 1 (subunit A1) of prolyl 4-hydroxylase and having a nucleic acid sequence of SEQ ID NO.: 28, and an expression vector comprising a polynucleotide sequence encoding a beta polypeptide (subunit B) of prolyl 4-hydroxylase and having a nucleic acid sequence of SEQ ID NO.: 30, and an expression vector comprising a polynucleotide sequence encoding HSP 47 and having a nucleic acid sequence of SEQ ID NO.: 24.

The host cells may further comprise a second expression vector comprising a polynucleotide sequence encoding human rCol7, having a nucleic acid sequence of SEQ ID NO.: 25.

In one preferred embodiment, the host cells of the present production system comprise a first collagen 7 expression vector comprising a polynucleotide sequence of SEQ ID NO.: 26, a second collagen 7 expression vector comprising a polynucleotide sequence of SEQ ID NO.: 27, an expression vector for expressing the alpha-1 polypeptide of human prolyl 4-hydroxylase comprising a polynucleotide sequence of SEQ ID NO.: 29 and an expression vector for expressing the beta polypeptide of human prolyl 4-hydroxylase comprising a polynucleotide sequence of SEQ ID NO.: 31. The engineered cells may further comprise an expression vector for expressing HSP 47 comprising a polynucleotide sequence of SEQ ID NO.: 32.

In some embodiments, the host cells of the present production system may be engineered to comprise an expression vector comprising a polynucleotide that encodes human rCol7, or a functional variant thereof, wherein the same expression vector further comprises a polynucleotide encoding an alpha polypeptide (subunit A) of human prolyl 4-hydroxylase or a functional variant thereof, and a polynucleotide encoding a beta polypeptide (subunit B) of human prolyl 4-hydroxylase or a functional variant thereof. In some examples, the same expression vector may further comprise a polynucleotide that encodes human HSP 47.

In some embodiments, the production system may comprise a plurality of homogenous engineered host cells that are derived from a single cell clone expressing human rCol7, or a functional variant thereof. In other embodiments, the production system may comprise a plurality of heterogenous engineered host cells that are derived from more than one cell clone expressing human rCol7 and/or functional variants thereof.

The host cells of the present production system may be any cell which is capable of expressing an exogenous polypeptide or protein of interest, e.g., rCol7. Host cells of the present disclosure may be eukaryotic cells, such as invertebrate (insect) cells or vertebrate cells, for instance Xenopus laevis oocytes or mammalian cells. In some embodiments, the host cells are mammalian cells including, but not limited to, fibroblasts, C127, VERO, HeLa, MDCK, CHO, COS, BHK, HEK293 cells and/or any cells derived from these mammalian host cells. The host cells may be primary cells or transformed cell lines. In one embodiment, an expression system for producing a collagen 7 composition comprises mammalian CHO cells, or cells derived from CHO cells.

TABLE 2
Expression constructs

| | | SEQ |
|---|---|---|
| Construct Name | Encoded polypeptide | ID NO.: |
| Collagen 7A sequence in the vector | Collagen 7, alpha chain | 25 |
| Puro_BT+_SLX-3631_Collagen 7A | Collagen 7, alpha chain | 26 |
| Hygro_BT+_SLX-3631_Collagen 7A | Collagen 7, alpha chain | 27 |
| hP4HA1 sequence in the vector | Prolyl hydroxylase, alpha polypeptide I | 28 |
| pBSK_ITR_CGAPD_hP4HA1_X29_ITR | Prolyl hydroxylase, alpha polypeptide I | 29 |
| hP4HB sequence in the vector | Prolyl hydroxylase, beta polypeptide | 30 |
| pBSK_ITR_CGAPD_hP4HB_X29_ITR | Prolyl hydroxylase, beta polypeptide | 31 |
| HSP 47 sequence in the vector | HSP 47 | 32 |

The host cells may be modified by any method known in the art. In one embodiment, host cells are modified by known methods for transfection of mammalian cells, including but not limited to, reagent-mediated methods (e.g., lipids, calcium phosphate, cationic polymers, DEAE-dextran, activated dendrimers and magnetic beads), electroporation, microinjection, laser faction and virus mediated methods. The transformed host cells may be stable cell clones selected through several rounds of selection process.

In some embodiments, the production system of the present disclosure further comprises a production reactor that may be any vessel suitable for the growth of the host cell culture for expressing rCol7 and/or functional variants thereof.

In some embodiments, the production system of the present disclosure can produce recombinant collagen 7 proteins for use as biopharmaceuticals. Particularly the host cells are stable for producing collagen 7 compositions comprising rCol7 and/or functional variants thereof. For example, the collagen 7 composition is produced from the engineered host cells at a quantity of more than 0.5 mg per liter culture, or more than 1 mg per liter culture, or more than 5 mg per liter culture, or more than 10 mg per liter culture, or more than 20 mg per liter culture, or more than 50 mg per liter culture. The terms “culture”, “cell culture” and “mammalian cell culture” as used herein refer to a mammalian cell population that is suspended in a medium under conditions suitable for survival and/or growth of the cell population.

The production reactor may be any size. The volume of the large-scale cell culture production reactor is typically at least 50 liters, or at least 100 liters, or at least 200 liters, or at least 300 liters, or at least 400 liters, or at least 500 liters, or may be 1,000, 2,500, 3,000, 4,000, 5000, 6,000, 7,000, 8,000, 9,000, 10,000, 12,000 liters or more, or any volume in between. The culture volume may be at least 500 mL, at least 1 liter and may be, 2, 3, 4, 5, 6, 7, 8, 9, 10, 100, 250, 300, 400, 500, 600, 700, 800, 900, 1,000, 2,500, 3,000, 4,000, 5,000, 6,000, 7,000, 8,000, 9,000, 10,000 liters or more, or any volume in between. The conditions of the production reactor may be controlled during the cell culturing period to ensure appropriate cell density and viability. These conditions include, but are not limited to, pH, temperature, humidity and CO2 supply.

In some embodiments, the production systems of the present disclosure can produce a collagen 7 composition, e.g., a level high enough such that it can be purified at a quantity of more than 1 mg per liter culture, or more than 5 mg per liter culture, or more than 10 mg per liter culture, or more than 20 mg per liter culture, or more than 50 mg per liter culture.

The host cells of the production system for producing collagen 7 compositions may be cultured using standard cell culture procedures and materials, which are known to those skilled in the art. In some embodiments, the host cells are cultured in serum free media. For example, the serum free medium may be a SFM2 medium that is animal-origin free. In some embodiments, the culture medium can further comprise at least one supplement, including L-Glutamine, thymidine and hypoxanthine, other nutrients such as lipids, amino acids, vitamins, and/or growth factors (e.g., HyClone Cell Boost 5 supplement provided by GE Healthcare).

In some embodiments, processes for culturing the host cells of the present production system may include methods for retaining the viability of the host cells. The maximum cell viability is desired. These methods may serve to minimize decreases in viable cell density and/or maintain high cell viability.

In some embodiments, the cell culture process involves double selection of cells positively expressing recombinant collagen 7 and/or functional variants thereof.

In another aspect of the disclosure, a method for producing a collagen 7 composition is provided, said method comprising: i) providing a polynucleotide encoding rCol7 or a functional variant thereof; ii) providing a polynucleotide encoding an alpha polypeptide 1 and a polynucleotide encoding a beta polypeptide of prolyl 4-hydroxylase or a functional variant thereof; iii) providing a polynucleotide encoding heat shock protein 47; iv) providing a cell expression system comprising host cells for producing rCol7 or functional variants thereof, the alpha polypeptide 1 and beta polypeptide of prolyl 4-hydroxylase or functional variants thereof, and HSP47; v) producing the collagen 7 composition by co-expressing the polynucleotides of (i), (ii) and (iii) in host cells of the cell expression system of (iv), and vi) collecting and purifying the produced collagen 7 composition.

In some embodiments, a method for producing a collagen 7 composition may comprise the steps: i) providing a vector for expressing rCol7 or a functional variant thereof, said vector comprising a polynucleotide encoding rCol7 or the functional variant thereof; ii) providing a vector for expressing an alpha polypeptide 1 (subunit A1) of prolyl 4-hydroxylase or a functional variant thereof, said vector comprising a polynucleotide encoding the alpha polypeptide 1 of prolyl 4-hydroxylase or the functional variant thereof; iii) providing a vector for expressing a beta polypeptide (subunit B) of prolyl 4-hydroxylase or a functional variant thereof, said vector comprising a polynucleotide encoding the beta polypeptide of prolyl 4-hydroxylase or the functional variant thereof; iv) providing a vector for expressing HSP 47, said vector comprising a polynucleotide encoding HSP 47 or a functional variant thereof; v) providing a cell expression system comprising host cells for producing rCol7 or functional variants thereof, the alpha polypeptide 1 and the beta polypeptide of prolyl 4-hydroxylase or functional variants thereof and HSP 47 or functional variants thereof; vi) producing the collagen 7 composition by co-expressing the vectors of (i), (ii), (iii) and (iv) in host cells of the cell expression system of (v), and vii) collecting and purifying the produced collagen 7 composition.

In some embodiments, the present method of preparing a collagen 7 composition further comprises: i) culturing host cells that are genetically modified to express rCol7 or functional variants thereof, in serum free media; and ii) recovering the rCol7, or functional variants thereof from the host cell culture.

The recombinantly produced collagen 7 compositions may be recovered from the culture medium by any methods known in the art, including, but not limited to, separating the host cells from the medium by centrifugation or filtration, viral inactivation, precipitating the proteinaceous components of the supernatant or filtrate by means of a salt, for instance ammonium sulphate, and removal of host cell nucleic acid contents. Optionally, the collagen 7 composition may be further purified. Purification may be achieved using any method known in the art, including, but not limited to, affinity chromatography, HPLC, ion exchange chromatography, hydrophobic interaction chromatography, size exclusion chromatography, protein A chromatography, Protein G chromatography, or the like.

In some embodiments, the downstream purification process may be designed to provide sufficient and effective virus clearance. In some examples, the process may include multiple unit operations dedicated to virus inactivation or removal. These unit operations can consistently and effectively. In the meantime, these unit operations do not have any adverse impact on product quality or cause significant yield loss. The downstream purification may remove any chemicals that are introduced in the process, such as those added in the dedicated virus clearance unit operations.

The downstream processes may also include concentration steps which may be intermingled with final ultrafiltration and diafiltration steps. Ultrafiltration (UF) is the process of separating extremely small particles and dissolved molecules from fluids. Ultrafiltration is typically used to separate proteins from buffer components for buffer exchange, desalting, or concentration and to remove sugars, non-aqueous solvents and materials of low molecular weight. The UF/DF step may be formed using high-performance membrane ultrafiltration, such as polyethersulfone ultrafiltration membranes.

In another aspect of the present disclosure, collagen 7 compositions produced by the present production systems, host cells and methods are provided. The collagen 7 composition prepared using the present system is correctly modified and functionally indistinguishable from naturally occurring collagen 7 protein. For example, the collagen 7 composition prepared by the present production system can incorporate into the anchoring fibrils within the basement membrane zone (BMZ) between the epidermis and dermis of skin. The collagen 7 composition prepared by the present production system may bind to laminin-332 and other collagen 7 binding partners. The collagen 7 composition may retain 20-100%, 50-100%, 50-90%, or at least 20%, 30%, 40%, 50%, 60%, 65%, 70%, 85%, 80%, 90%, 95% or 100% of the functions and/or activities of wild-type human collagen 7 protein.

In one embodiment, a collagen 7 composition is obtainable by a method comprising an in vitro cell expression system for production of rCol7 or functional variants as described elsewhere herein.

The collagen 7 composition may comprise a plurality of recombinantly expressed collagen 7 alpha chain polypeptides, or a plurality of a functional variants of collagen 7 alpha chain polypeptides, or a plurality of functionally equivalent collagen 7 alpha chain polypeptides, or a mixture thereof. As a non-limiting example, a collagen 7 composition prepared by the present cell expression system may comprise a mixture of a plurality of recombinantly expressed collagen 7 alpha chain polypeptides that comprise the wild type collagen 7 polypeptide and a plurality of functionally equivalent collagen 7 alpha chain polypeptides, for example, a polypeptide comprising one or more amino acid substitutions (e.g., D1033Y).

### Pharmaceutical Compositions and Formulations

In one aspect of the present disclosure, the collagen 7 compositions comprising human rCol7, or functional variants thereof, produced by the present expression systems and host cells, may be formulated as a pharmaceutical composition. The pharmaceutical composition may further comprise at least one pharmaceutically acceptable carrier. The pharmaceutical formulations and compositions are construed for administration of a therapeutically effective amount of the collagen 7 composition of the present disclosure to a subject, in order to prevent, alleviate and/or reduce the symptoms of a skin condition, such as a skin symptom associate with DEB, e.g., a skin wound. Pharmaceutical compositions may take the form of any acceptable pharmaceutical formulation suitable for an intended mode of administration and therapeutic application.

The pharmaceutical composition may be formulated as a solution or suspension used for parenteral, intradermal, or subcutaneous application. The pharmaceutical composition may be formulated so as to be suitable for injection. The injectable formulation may be sterile, including, but not limited to sterile aqueous solutions or dispersions and sterile powders for the extemporaneous preparation of sterile injectable solutions or dispersions. The pharmaceutical composition may be formulated for oral administration and may be in the form of tablets, pills, capsules, troches, powders and the like. The pharmaceutical composition may be formulated for topical administration, such as creams, hydrogels and the like.

Other formulation forms include, but are not limited to, a liquid, a semi-solid or solid dosing form, a hydrogel, a cream, a liquid solution (e.g., injectable liquid solution), a dispersion or suspension, a powder, and a liposome.

Pharmaceutical formulations are stable under the conditions of manufacture and storage and will be preserved against the contaminating action of microorganisms such as bacteria and fungi. Prevention of the contamination of microorganisms can be achieved by various antibacterial and antifungal agents, for example, parabens, chlorobutanol, phenol, ascorbic acid, thimerosal, and the like.

In some embodiments, the pharmaceutical composition comprises an active drug ingredient including recombinant collagen 7, and/or functional variants thereof, and/or a mixture thereof, at a concentration ranging from 0.1 mg/mL to 200 mg/mL, or from 1 mg/mL to 200 mg/mL, or from 1 mg/mL to 10 mg/mL, or from 10 mg/mL to 200 mg/mL, or from 10 mg/mL to 100 mg/mL, or from 10 mg/mL to 50 mg/mL. In one embodiment, the collagen 7 composition included in the formulation of the present disclosure has a given concentration, including, for example, a concentration of at least about 0.1 mg/mL, at least about 1 mg/mL, at least about 2 mg/mL, at least about 5 mg/mL, at least about 10 mg/mL, at least about 15 mg/mL, at least about 20 mg/mL, at least about 25 mg/mL, at least about 30 mg/mL, at least about 40 mg/mL, at least about 50 mg/mL, at least about 75 mg/mL, $at least about 100 mg/mL, at least about 125 mg/mL, at least about 150 mg/mL, at least about 175 mg/mL, $t least about 200 mg/mL, or greater than about 200 mg/mL, or greater than about 300 mg/mL, or greater than about 400 mg/mL, or greater than about 500 mg/mL.

The pharmaceutical composition may comprise a collagen 7 composition that essentially retains the physical and/or chemical stability and/or biological activity upon storage. The stability of a protein may be assessed using any analytical techniques available in the art. For example, the stability of collagen 7 may be determined according to the percentage of monomer protein in the solution, with a low percentage of degraded (e.g., fragmented) and/or aggregated protein. For example, a pharmaceutical composition comprising a stable collagen 7 protein may include about 60% to 99% monomer protein, or about 70% to 80% monomer protein. In some examples, the pharmaceutical composition comprising a stable collagen 7 protein may include at least 95% monomer protein, or at least 90% monomer protein, or at least 85% monomer protein, or at least 80% monomer protein, or at least 75% monomer protein, or at least 70% monomer protein, or at least 65% monomer protein. Alternatively, a pharmaceutical composition of the disclosure may include no more than 5% aggregate and/or degraded protein.

In some embodiments, the collagen 7 composition comprises a mixture of naturally occurring collagen 7 and at least one functional variant thereof. In some embodiments, the collagen 7 compositions are produced and purified using the present production system comprising host cells genetically engineered to express recombinant collagen 7 and/or functional variants thereof.

In some embodiments, the pharmaceutical composition comprises at least one pharmaceutically acceptable carrier, for example, an excipient, a surfactant, a buffering system, a stabilizing agent that stabilizes the collagen 7 composition, a tonicity modifier, an anti-oxidant, a cryoprotectant, a bulking agent, a lyroprotectant, a basic component or an acidic component, and the like.

As used herein, the term “excipient” refers to an agent that may be added, for example, to a pharmaceutical formulation to provide a desired consistency, to improve stability and solubility, and/or to adjust osmolality, and/or to adjust other features that fit the purpose of usage of the pharmaceutical composition. $ Examples of commonly used excipients include, but are not limited to, sugars, polyols, amino acids, surfactants, and polymers. In some examples, the excipient may be an ionic excipient or non-ionic excipient. The ionic excipient has a net charge under certain formulation conditions, such as pH. Examples of an ionic excipient include, but are not limited to, histidine, arginine, and sodium chloride. The non-ionic excipient has no net charge under certain formulation conditions, such as pH. Examples of non-ionic excipients include, but are not limited to, sugars (e.g., sucrose), sugar alcohols (e.g., mannitol), and non-ionic surfactants (e.g., polysorbate 80).

As used herein, the term “stabilizing agent” refers to an excipient that improves or otherwise enhances stability. Stabilizing agents include, but are not limited to, α-lipoic acid, α-tocopherol, ascorbyl palmitate, benzyl alcohol, biotin, bisulfites, boron, butylated hydroxyanisole (BHA), butylated hydroxytoluene (BHT), ascorbic acid and its esters, carotenoids, calcium citrate, acetyl-L-carnitine, chelating agents, chondroitin, chromium, citric acid, coenzyme Q-10, cysteine, cysteine hydrochloride, 3-dehydroshikimic acid (DHS), EDTA (ethylenediaminetetraacetic acid; edetate disodium), ferrous sulfate, folic acid, fumaric acid, alkyl gallates, garlic, glucosamine, grape seed extract, gugul, magnesium, malic acid, metabisulfite, N-acetyl cysteine, niacin, nicotinomide, nettle root, ornithine, propyl gallate, pycnogenol, saw palmetto, selenium, sodium bisulfite, sodium metabisulfite, sodium sulfite, potassium sulfite, tartaric acid, thiosulfates, thioglycerol, thiosorbitol, tocopherol and their esters, e.g., tocopheral acetate, tocopherol succinate, tocotrienal, d-α-tocopherol acetate, vitamin A, B, C, D, or E and their esters, e.g., vitamin E acetate, zinc, and combinations thereof.

As used herein, the term “surfactant” may refer to an agent that can protect collagen 7 protein from any interface-induced stress. Examples of surfactants may include, but are not limited to, polysorbates (e.g., Polysorbate 20, Polysorbate 80), polyoxyethylene alkyl ethers, poloxamer such as Tween 20, Tween 80, or poloxamer 188. Poloxamer 407. Other compounds that can protect the collagen 7 compositions may include sugars, such as sucrose, glucose, trehalose, mannitol, mannose, and lactose; polymers, such as dextran, hydroxyethyl starch and polyethylene glycol; and amino acids, such as glycine, arginine (e.g., L-arginine), leucine, and serine.

In some embodiments, the pharmaceutical composition may further comprise a buffer system, an acidic component or a basic component. The buffer may be, but is not limited to, a phosphate buffer (e.g., PBS), an acetate buffer, or a Tris buffer. Examples of acidic components include phosphoric acid, hydrochloric acid, acetic acid, citric acid, oxalic acid, succinic acid, tartaric acid, lactic acid, malic acid, glycolic acid and fumaric acid. Examples of basic components include potassium hydroxide (KOH) and sodium hydroxide (NaOH). The acidic components and basic components are used to adjust the pH of the formulation.

As used herein, the term “antioxidant” refers to an agent that inhibits oxidation and thus is used to prevent the deterioration of preparations due to the oxidative process. $ Examples of antioxidants may include, but are not limited to, acetone, sodium bisulfate, ascorbic acid, ascorbyl palmitate, citric acid, butylated hydroxyanisole, butylated hydroxytoluene, hydrophosphorous acid, monothioglycerol, propyl gallate, methionine, sodium ascorbate, sodium citrate, sodium sulfide, sodium sulfite, sodium bisulfite, sodium formaldehyde sulfoxylate, thioglycolic acid, sodium metabisulfite, EDTA (edetate), pentetate and others known to those of ordinary skill in the art.

Other pharmaceutically acceptable carriers, excipients, or stabilizers, such as those described in Remington: The Science and Practice of Pharmacy 20th edition, Gennaro, Ed., Lippincott Williams & Wilkins (2000) may also be included in a collagen 7 formulation described herein, provided that they do not adversely affect the desired characteristics of the formulation.

In some embodiments, the pharmaceutical composition may further comprise one or more active agents for skin treatment.

In some embodiments, the pharmaceutical composition of the present disclosure has decreased immunogenicity.

In one aspect of the present disclosure, the pharmaceutical composition comprising the collagen 7 composition may be formulated as a liquid solution, for example as an aqueous liquid solution. The term “aqueous” as used herein refers to a water-based protein formulation, but may optionally contain additional solvents, e.g., a small amount of a water-miscible solvent. As a non-limiting example, the pharmaceutical composition is a stable liquid solution.

In some embodiments, the pharmaceutical formulation comprising a collagen 7 composition is injectable. The injectable collagen 7 composition in the present method may further contain diluents, solubilizing agents, pH-modifiers, buffers, sulfur-containing reducing agents, antioxidants, preservatives or the like, if desired. Buffers used in the present injectable composition may include acids commonly used as buffers in injections and salts thereof or mixed solutions with a base or a salt thereof, such as phosphoric acid, acetic acid, hydrochloric acid, phthalic acid, boric acid, citric acid, carbonic acid, succinic acid and salts thereof, preferably phosphate buffers (sodium mono-hydrogen phosphate-sodium dihydrogen phosphate system) and/or citrate buffers and/or acetate buffers. The concentration of buffers used in the injectable collagen 7 composition may be 0-300 mM, or 0 to 100 mM, or 10 to 200 mM, or 30 to 250 mM, preferably 0-100 mM on the basis of the total amount of the injectable composition. The pH of the present formulation and the injectable composition may be 6.5-7.4, preferably 6.8-7.2.

In some embodiments, the formulation of the present disclosure is suitable for any use, such as in vitro and/or in vivo use. The formulation may be suitable for administration to a subject via any mode of administration, including, but not limited to, subcutaneous, intravenous, inhalation, intradermal, transdermal, intraperitoneal, and intramuscular administration. The formulation of the disclosure may be used to treat a skin disease (e.g., RDEB) in a subject.

In some embodiments, the pharmaceutical formulation comprising a collagen 7 composition is particularly well-suited for a single dose formulation or for multidose formulations. A multidose formulation is a formulation having more than one dose of the therapeutic collagen 7 composition. The healthcare provider and/or patient can administer a single dose from the multidose formulation, storing the remainder of the formulation for future administration in one or more subsequent doses. The number of doses in the multidose formulations disclosed herein can be about 2 to about 50, preferably about 2 to about 40, and more preferably about 2 to about 25. Also contemplated are doses of at least 5, at least 10, and at least 20. Specific doses include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 doses of the formulation.

### Administration and Dosing

According to the present disclosure, pharmaceutical compositions and formulations comprising collagen 7 compositions produced by the present production system may be administered to a subject in need by any appropriate route known in the art including, but not limited to, oral, parenteral (including intra-arterial, intravenous, subcutaneous, intraperitoneal and intramuscular) injection or infusion, airway (aerosol), nasal, rectal, intratracheal, pulmonary, e.g., by inhalation or insufflation of powders or aerosols, including by nebulizer, subdermal, e.g., via an implanted device, intracranial, (e.g., intraparenchymal), epidermal, topical (including dermal, transdermal, transmucosal, buccal, sublingual, and intraocular), vaginal, transmucosal, bronchial, and ophthalmic administration. Pharmaceutical compositions and/or formulations of the disclosure may be administered by more than one route, depending upon whether local or systemic treatment is desired and/or upon the skin area to be treated. More than one route can be used concurrently, if desired.

In some embodiments, the administration route may local, such as to the local area of the skin or eye. In other embodiments, the administration route may be systemic, for example, injection or infusion.

The pharmaceutical compositions in accordance with the disclosure are typically formulated in dosage unit form for ease of administration and uniformity of dosage. It will be understood, however, that the total daily usage of the compositions of the present disclosure may be decided by the attending physician within the scope of sound medical judgment. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual pharmaceutical compositions encompassed by the disclosure can be made using conventional methodologies or on the basis of in vivo testing using an appropriate animal model. For example, in some embodiments, an appropriate dose or amount, is a dose or amount sufficient to reduce a disease severity index score by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100% or more.

The formulations and dosages described herein are designed to maximize clinical efficacy in the treatment of diseases and disorders while simultaneously decreasing or minimizing adverse side effects.

In some embodiments, the present composition is administered in a therapeutically effective amount and/or according to a dosing regimen that is correlated with a particular desired outcome (e.g., preventing and/or treating epidermolysis bullosa). For example, in some embodiments, a therapeutically effective dosage amount of a collagen 7 composition may be an amount in the range of 0.1 mg to 1,000 mg (e.g., about 1 mg to 1,000 mg, 10 mg to 1,000 mg, 20 mg to 1,000 mg, 30 mg to 1,000 mg, 40 mg to 1,000 mg, 50 mg to 1,000 mg, 60 mg to 1,000 mg, 70 mg to 1,000 mg, 80 mg to 1,000 mg, 90 mg to 1,000 mg, 100 mg to 1,000 mg, 200 mg to 1,000 mg, 10 mg to 900 mg, 10 mg to 800 mg, 10 mg to 700 mg, 10 mg to 600 mg, 10 mg to 500 mg, 100 mg to 1,000 mg, 100 mg to 900 mg, 100 mg to 800 mg, 100 mg to 700 mg, 100 mg to 600 mg, 100 mg to 500 mg, 100 mg to 400 mg, 100 mg to 300 mg, 200 mg to 900 mg) per kilogram body weight of the subject. In other embodiments, a therapeutically effective dosage amount may be, for example, about 0.001 mg/kg to 500 mg/kg, e.g., from about 0.001 mg/kg to 400 mg/kg, from about 0.001 mg/kg to 300 mg/kg, from about 0.001 mg/kg to 200 mg/kg, from about 0.001 mg/kg to 100 mg/kg, from about 0.001 mg/kg to 90 mg/kg, from about 0.001 mg/kg to 80 mg/kg, from about 0.001 mg/kg to 70 mg/kg, from about 0.001 mg/kg to 60 mg/kg, from about 0.001 mg/kg to 50 mg/kg, from about 0.001 mg/kg to 40 mg/kg, from about 0.001 mg/kg to 30 mg/kg, from about 0.001 mg/kg to 25 mg/kg, from about 0.001 mg/kg to 20 mg/kg, from about 0.001 mg/kg to 15 mg/kg, from about 0.001 mg/kg to 10 mg/kg.

The total dosage may be administered in a single dose, multiple doses, repeated doses, as a continual dose or a combination thereof. In some embodiments, pharmaceutical compositions of the present disclosure may be administered in a single daily dose, or the total daily dosage may be administered in divided doses of two, three or four times daily.

The effect of a single dose on any particular phenotype or symptom can be long lasting, such that subsequent doses are administered at not more than 3, 4, or 5 days intervals, or at not more than 1, 2, 3, or 4 weeks intervals, or at not more than 1, 2, 3, or 4 months intervals.

In some embodiments, pharmaceutical compositions and formulations comprising collagen 7 compositions produced by the present production system may be administered to a patient in need for the remainder of their lifetime. The timing intervals of administration and dosage for each administration can be adjusted according to the patient's condition (e.g., a skin condition). In one example, pharmaceutical compositions and formulations may be chronically administered. Chronic administration can include the administration of more than one dose of an agent over a period of time, e.g., for the duration of the lifetime of a subject. The concentration of collagen 7 composition may be maintained at a therapeutically or prophylactically effective level throughout the course of treatment.

In some embodiments, the period of time of chronic administration may include, but is not limited to, at least 1 month, at least 2 months, at least 3 months, at least 6 months, at least 1 year, at least 2 years, at least 3 years, at least 4 years, at least 5 years, at least 10 years, at least 15 years, at least 20 years, at least 25 years, at least 30 years, at least 35 years, at least 40 years, at least 45 years, at least 50 years, at least 55 years, at least 60 years, at least 65 years, at least 70 years, at least 75 years, at least 80 years, at least 85 years, at least 90 years or at least 100 years, or any time period between 1 month and 100 years.

For example, the dosing timing may include, once daily, or once weekly, or once every other week, or once monthly, or once every other month, or once every three months, or once every 6 months, or once every 12 months, or once every 18 months, or once every 24 months, or once every two years, or once every 5 years. The pharmaceutical composition may be administrated twice per week, twice per month, or twice every other month, or twice every three months, or twice every 6 months, or twice every 12 months, or twice every 18 months, or twice every 24 months.

As a non-limiting example, the chronic administration can include a series of doses which together provide an effective amount for alleviating at least one symptom associated with EB, particularly DEB (e.g., DDEB and RREB). Chronic administration can include a series of doses which, in combination, provide an effective amount for treating, preventing, preventing the progression of, or delaying the onset of EB, particularly DEB, such as DDEB or RDEB. The dosing timing may be tailored for a subject, depending on several factors, including the types of EB, such as DEB, DDEB or RDEB, the presence of EB related symptoms, the age, body weight, general health, sex and diet of the patient; the time of administration, route of administration, and rate of excretion of the specific composition employed; the duration of the treatment; drugs used in combination or coincidental with the collagen 7 composition; and like factors well known in the art.

Applications of the rCol7 Composition

In accordance with the present disclosure, collagen 7 compositions produced by the present production systems, and pharmaceutical compositions or formulations thereof may be used for replacing the collagen 7 substance in a subject, particularly in the skin of the subject. The collagen 7 can subsequently localize to the BMZ of the skin and form anchoring fibrils.

### Restoration of Collagen 7 Levels

In one aspect of the disclosure, collagen 7 compositions produced by the host cells and production systems of the present disclosure and pharmaceutical compositions and formulations thereof may be used to restore collagen 7 to a functional level in a subject in need, by restoring collagen 7 function to within a range of 20% to 100%, 30% to 100%, 40% to 100%, 50% to 100%, 60% to 100%, 70% to 100%, 80% to 100%, 90% to 100%, 30% to 90%, 40% to 90%, 50 to 90%, 60-90%, or 70% to 90% of the normal functional level of wild type collagen 7 in the subject.

In some embodiments, the collagen 7 composition may restore the anchoring fibrils of the skin, holding the epidermal layer and the dermal layer of the skin together.

### Therapeutic Uses

In one aspect of the disclosure, pharmaceutical compositions and formulations comprising a collagen 7 composition may be utilized for treatment of a skin condition, e.g., a skin symptom associated with epidermolysis bullosa (EB). Methods for treating a subject having a skin disorder comprise administering to a subject having a skin disorder a pharmaceutical formulation comprising a collagen 7 composition, wherein the composition is administered systemically to the subject (e.g., injection or infusion). The collagen 7 composition may prevent, inhibit, alleviate or inhibit the progression of a skin symptom of the disorder, e.g., skin wound, blistering and scarring, etc.

The skin disorder may be a genetic disorder, such as epidermolysis bullosa (EB), caused by genetic mutations. Epidermolysis bullosa is a group of inherited genetic conditions in which the skin is very fragile and can blister easily because of a lack of anchoring proteins holding the epidermal and dermal layers of the skin together. Blisters and skin erosions form spontaneously and in response to minor injury or friction, such as rubbing, scratching, or trivial trauma. Furthermore, as a complication of chronic skin damage, patients suffering from EB have an increased risk of malignancies (cancers) of the skin. Over 300 mutations of anchoring proteins have been identified in EB diseases. An EB disease may include, but is not limited to, epidermolysis bullosa simplex, junctional epidermolysis bullosa, dystrophic epidermolysis bullosa, epidermolysis bullosa (lethal acantholytic) and epidermolysis bullosa acquisita. Dystrophic epidermolysis bullosa (DEB) (dominant or recessive DEB) caused by mutations in the COL7A1 gene encoding Type VII collagen (collagen 7), is one of the most common forms of epidermolysis bullosa. The symptoms of this condition vary widely among affected individuals. In mild cases, blistering may primarily affect the hands, feet, knees, and elbows. Severe cases of this condition involve widespread blistering and scarring that can lead to vision loss, disfigurement, and other serious medical problems.

In some embodiments, pharmaceutical compositions and formulations comprising a collagen 7 composition may be used for preventing, preventing the progression of, or delaying the onset of one or more symptoms associated with DEB, including dominant DEB (DDEB) and recessive DEB (RDEB). The symptoms associated with an EB may include, but are not limited to, a skin condition such as thin and dry skin, open wounds (e.g., chronic and non-healing wounds), blistering (mild or severe), scarring, infection caused by a chronic wound (secondary skin infection) or a skin cancer (e.g., squamous cell carcinoma); constricted esophagus such as chronic scarring, webbing, and obstruction of the esophagus; contractures such as flexion contractures (e.g., of the extremities); pseudosyndactyly of the hands or feet; urethral lesions (e.g., urethral stenosis); mucosal lesions; lesions of squamous epithelial tissue; lesions of the gastrointestinal tract such as rectal or anal lesions; bulla formation such as bulla formation post manual trauma; nail or teeth deformities; eye disorders such as blepharitis and corneal scarring; anemia, malnutrition; sepsis; hoarse voice; phimosis; malabsorption; allergies and immunodeficiencies (e.g., increased frequencies of asthma, allergies, eczema or rhinitis symptoms); and failure to thrive.

In some embodiments, the treatment with pharmaceutical compositions of the present disclosure may result in amelioration of one or more symptom associated with DEB in the range of 20% to 100%, or 30% to 100%, or 35% to 100%, or 40% to 100%, or 45% to 100%, or 45% to 100%, or 50% to 100%, or 55% to 100%, or 60% to 100%, or 65% to 100%, or 70% to 100%, or 75% to 100%, or 80% to 100%, or 85% to 100%, or 90% to 100%, compared to a non-treated patient.

In some embodiments, pharmaceutical compositions and formulations of the present disclosure may be used for treatment of other skin diseases, including, but not limited to, non-healing wounds, skin wounds resulting from a skin cancer, skin wounds from diabetes such as type II diabetes, chronic skin wounds in aged individuals, open wounds, skin wounds from allergic reaction, surgical wounds, wounds caused by injury, wounds due to limited mobility of the subject, wounds associated with organ transplantation, and other damages such as exposure to sun, wind, heat and cold, etc.

Skin cancers may include but are not limited to, actinic keratosis, atypical moles, basal cell carcinoma, melanoma (e.g., superficial spreading melanoma, nodular melanoma, lentigo maligna melanoma, acral lentiginous melanoma), merkel cell carcinoma, squamous cell carcinoma, dermatofibrosarcoma, cutaneous lymphoma and atypical fibroxanthoma.

Wounds in aged individuals may be chronic and non-healing. Age related disorders may include skin cancer, diabetes and others.

Allergic reaction may cause a significant skin reaction, ranging from mild to severe. Common symptoms from long-term allergic reaction may include eczema.

### Combination Therapies

In some embodiments, the present disclosure encompasses administration of a pharmaceutical composition comprising a collagen 7 composition together with one or more additional agents, as a part of a combination therapy. The collagen 7 composition of the present disclosure may be administered prior to, concurrently with, or subsequent to one or more additional therapies. In one embodiment, it is contemplated that any known therapy or therapeutic for the treatment of epidermolysis bullosa or for the amelioration of a clinical condition associated with EB, may be used with the present collagen 7 composition.

Exemplary additional agents and therapies may include, but are not limited to, antibiotics, analgesics, opioids, anti-virals, anti-inflammatory agents, oral steroids, nutritional supplements, or topical creams that help to manage pain and itching.

Antibiotics can include, but are not limited to, Aknilox, Ambisome, Amoxycillin, Ampicillin, Augmentin, Avelox, Azithromycin, Bactroban, Betadine, Betnovate, Blephamide, cancidas, Cefaclor, Cefadroxil, Cefdinir, Cefepime, Cefix, Cefixime, Cefoxitin, Cefpodoxime, Cefprozil, Cefuroxime, Cefzil, Cephalexin, Cephazolin, Ceptaz, Chloramphenicol, Chlorhexidine, Chloromycetin, Chlorsig, Ciprofloxacin, Clarithromycin, Clindagel, Clindamycin, Clindatech, Cloxacillin, Colistin, Co-trimoxazole, Demeclocycline, Diclocil, Dicloxacillin, Doxycycline, Duricef, Erythromycin, Flagyl alcohol, Flagyl dosage, Flagyl pregnancy, Flagyl side effects, Flagyl treatment, Flamazine, Floxin, Framycetin, Fucidin, Furadantin, Fusidic, Gatifloxacin, Gemifloxacin, Gemifloxacin, Ilosone, Iodine, Levaquin, Levofloxacin, loceryl, Lomefloxacin, Maxaquin, Mefoxin, Meronem, Minocycline, Moxifloxacin, Myambutol, Mycostatin, Neosporin, Netromycin, Nitrofurantoin, Norfloxacin, Norilet, Ofloxacin, Omnicef, Ospamox, Oxytetracycline, Paraxin, Penicillin, Pneumovax, Polyfax, Povidone, Rifadin, Rifampin, Rifaximin, Rifinah, Rimactane, Rocephin, Roxithromycin, Seromycin, Soframycin, Sparfloxacin, Staphlex, Targocid, Tetracycline, Tetradox, Tetralysal, tobramycin, Tobramycin, Trecator, Tygacil, Vancocin, Velosef, Vibramycin, Xifaxan, Zagam, Zitrotek, Zoderm, Zymar, and Zyvox.

Anti-viral agents can include, but are not limited to, Abacavir, Aciclovir, Acyclovir, Adefovir, Amantadine, Amprenavir, Ampligen, Arbidol, Atazanavir, Atripla, Boceprevir, Cidofovir, Combivir, Darunavir, Delavirdine, Didanosine, Docosanol, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Oseltamivir, Peginterferon alfa-2a, Penciclovir, Peramivir, Pleconaril Podophyllotoxin, Protease inhibitor, Raltegravir, Reverse transcriptase inhibitor, Ribavirin, Rimantadine, Ritonavir, Pyramidine, Saquinavir, Stavudine, Tea tree oil, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, and Zidovudine.

Anti-inflammatory agents can include, but are not limited to, Ibuprofen, Naproxen, Aspirin, Diclofenac, Indomethacin, Ketoprofen, Piroxicam, Meloxicam, Sulindac, and Steroids.

Nutritional supplements can include, but are not limited to, iron, calcium, vitamin D, selenium, carnitine, and zinc.

Other combined therapies may include, but are not limited to, surgery to correct abnormal motion, e.g., surgery to correct fusing of finger or toes or abnormal bends in the joints, surgical dilation of the esophagus to improve the ability to eat, skin grafts, gene therapy, cell-based therapy (e.g., transplant of fibroblasts engineered to express collagen 7 or functional variants), bone marrow transplantation, other protein replacement therapy, and/or combinations thereof.

### EQUIVALENTS AND SCOPE

Those skilled in the art will recognize or be able to ascertain using no more than routine experimentation, many equivalents to the specific embodiments in accordance with the disclosure described herein. The scope of the disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### EXAMPLES

### Example 1: Polypeptide Expression Constructs

Plasmid constructs that are used to express collagen 7 alpha-chain polypeptide and other polypeptides that increase expression of collagen 7 in cells were constructed following standard molecular techniques. The detailed methods to generate these expression constructs are described below.

### Collagen 7 Expression Constructs

GFP expression plasmids with either puromycin resistance gene (Puro-) (pSVpuro-C+_EF1alpha (KOZAK-ext9) EGFP_BGHpA>X-S*29) or hygromycin resistance gene (Hygro-) (pSVhygro-C+_EF1alpha (KOZAK-ext9) EGFP_BGHpA>X_29) were digested with restriction enzymes HindIII and Xbal. The resulting two DNA fragments were then separated by electrophoresis and the vector fragment from each construct was cut out of the gel, transferred into a 1.5 mL microtube and purified using standard techniques. A 9114 bp Puro-vector band and a 9552 bp Hygro-vector band were recovered from the puro-construct and hygro-construct, respectively.

The polynucleotide encoding collagen 7 alpha-chain (collagen 7A) was excised from the GeneArt plasmid 11AAER3P_Collagen 7A_pMA by cutting the plasmid with HindIII and Xbal. The resulting two DNA fragments were separated by electrophoresis and an 8870 bp band corresponding to Collagen 7 alpha-chain (SEQ ID NO.: 25) was recovered and purified using standard techniques.

The purified 8870 bp collagen 7A fragment was assembled with the 9114 bp vector fragment to create Puro_BT+_SLX-3631_Collagen 7A (SEQ ID NO.: 26) or assembled with the 9552 bp vector fragment to create Hygro_BT+_SLX-3631_Collagen 7A (SEQ ID NO.: 27). The constructs were prepared by ligating the purified vector fragment, (the 9114 bp Puro vector fragment or the 9552 bp Hygro vector fragment) with the 8870 bp collagen 7A fragment using LigaFast Rapid DNA Ligation System (Promega, Cat. No.: M8221) in a final volume of 10 μL for 5 min at RT, following manufacturer's instructions. The ligation mixtures were then used to transform 50 μL of competent DH5 alpha cells (Invitrogen, Cat. No.: 18265-017) per manufacturer's instructions.

The integrity and structure of the collagen 7A expression plasmids were confirmed by restriction analysis. One bacterial clone was expanded in 150 mL LB media with 100 μg/mL ampicillin and proteins were extracted. The Collagen 7A constructs were linearized using PvuI-HF (NEB, Cat. No.: R3150L) overnight at 37° C. and digested with restriction enzymes. The digested DNA was quantified and separated by electrophoresis. Three bands (15426 bp, 1512 bp and 1046 bp) for the construct Puro BT+SLX-3631_Collagen 7A (SEQ ID NO.: 26) were present as expected and three bands (15864 bp, 1512 bp and 1046 bp) for the construct Hygro_BT+_SLX-3631_Collagen 7A (SEQ ID NO.: 27), were present as expected.

### Prolyl 4-Hydroxylase Expression Constructs

To create the hP4HA1 construct, the GFP expression plasmid pBSK_ITR_CGAPD_EGFP_X29_ITR was digested by HindIII to excise the GFP sequence. The digested DNA was purified as described above. The purified DNA was filled using DNA polymerase (Roche) and purified using standard techniques, then digested with Fsel.

The two DNA bands from the HindIII/Fsel double digestion were separated by electrophoresis. The 8584 bp band corresponding to the vector was recovered and purified using standard techniques.

The human prolyl 4-hydroxylase, alpha polypeptide I (hP4HA1_NM_000917) was amplified by PCR using the forward primer hP4HA1_Fw_HindIIIfilled

(TACCGCCACCATGATCTGGTATATA TTAATTATAGGAATTCTGCT; SEQ ID NO.: 33), the reverse primer hP4HA1_Rv_Fsel (TCATGGCCGGCCGCCCCGACTTATCATTCCAATTCTGACAACGTACAA; SEQ ID NO.: 34) and cDNA of human normal tissues (Biochain Institute, No.: B110179) as template. The 1638 bp band corresponding to human P4HA1 was recovered and purified using standard techniques. The purified 1638 bp PCR product was digested with Fsel and further purified.

The 1632 bp hP4HA1 fragment (SEQ ID NO. 28) was assembled with the 8584 bp vector fragment to create pBSK_ITR_CGAPD_hP4HA1_X29_ITR (SEQ ID NO.: 29). The purified hP4HA1 and vector fragments were ligated. The whole ligation mixture was used to transform 50 μL of competent DH5 alpha cells following the manufacturer's instructions. The integrity and structure of the newly created plasmid was checked by restriction analysis as described above.

A sample of the hP4HA1 construct pBSK_ITR_CGAPD_hP4HA1 X29 ITR was linearized with PvuI-HF and further verified by digestion with Xbol and Xbal. The digested DNA was quantified and separated by electrophoresis. Two fragments (8780 bp and 1440 bp) were present, as expected.

To create the hP4HB construct, the GFP expression plasmid pBSK_ITR_CGAPD_EGFP_X29 ITR was digested with HindIII and Xbal. The 8603 bp fragment corresponding to the vector was recovered and purified as described above.

The human prolyl 4-hydroxylase, beta polypeptide (hP4HB_NM_000918) was amplified by PCR using the forward primer hP4HB_Fw HindIII (TCCCMGCTTACCGCCACCATGCTGCGCCGCGCTCT; SEQ ID NO.: 35), the reverse primer hP4HB Rv Xbal (CTAGTCTAGATTATCACAGTTCATCTTTCACAGCTTTCTGA; SEQ ID NO.: 36) and cDNA of human normal tissues as template. The 1559 bp PCR fragment was purified and digested by HindIII and Xbal.

The resulting 1545 bp hP4HB fragment (SEQ ID NO.: 30) was assembled with the 8603 bp vector fragment to create pBSK_ITR_CGAPD_hP4HB_X29_ITR (SEQ ID NO.: 31). The purified hP4HB and vector fragments were ligated and used to transform 50 μL of competent DH5 alpha cells. The integrity and structure of the newly created plasmid were checked by restriction analysis as described above.

A sample of the hP4HB construct pBSK_ITR_CGAPD_hP4HB_X29_ITR was linearized with PvuI-HF and verified by digestion with Xbal and HindIII-HF. The digested DNA was quantified and separated by electrophoresis. Two fragments (8603 bp and 1545 bp) were present, as expected.

HSP47, hPEPD and COMSC Expression Constructs

Similar methods were used to generate a construct to express HSP 47. The GFP expression plasmid pBSK_ITR_BT+_EGFP_X29_ITR was digested. The fragment corresponding to the vector was recovered and purified using standard technologies. A nucleic acid sequence encoding human HSP 47 (SEQ ID NO.: 32) was inserted to the vector fragment to generate the HSP 47 expressing construct (pBSK ITR BT+ SHSP47 X29 IT). The integrity and structure of the plasmid was checked by restriction analysis as described previously and lastly, the plasmid was quantified.

Human prolidase-encoding sequence hPEPD (NM_000285) was amplified by PCR using the forward primer hPEPD_Fw_HindIII (TCCCAAGCTTACCGCCACCATGGCGGCGGCCACCGGA; SEQ ID NO.: 37), reverse primer hPEPD_Rv_Xbal (CTAGTCTAGATTATCACTTGGGGCCAGAGAAGGGGGT; SEQ ID NO.: 38) and cDNA of human normal tissues as template. The 1514 bp PCR product was purified and digested by HindIII and Xbal. The recovered 1500 bp hPEPD fragment was assembled with the 8930 bp vector fragment to create pBSK_ITR_BT+_hPEPD_X29_ITR. The purified hPEPD and vector fragments were ligated together and used to transform 50 μL of competent DH5 alpha cells. The integrity and structure of the newly created plasmid was checked by restriction analysis as described previously and lastly, the plasmid was quantified

Human CIGALT1-specific chaperon 1 encoding sequence (NM_001011551) was amplified by PCR using the forward primer COSMC_Fw_HindIII filled (TACCGCCACCATGCTTTCTGAAAGCAGCTCCTT; SEQ ID NO. 39), reverse primer COSMC_Rv_Xbal (CTAGTCTAGATTAGTCATTGTCAGAACCATTTGGAGGT; SEQ ID NO.: 40) and cDNA of human normal tissues has template. The 977 bp PCR product was purified and digested by Xbal. The recovered 968 bp hCOSMC fragment was assembled with the 8930 bp vector fragment (cut from the pBSK_ITR_BT+_EGFP_X29_ITR plasmid using HindIII) to create pBSK_ITR_BT+hCOSMC_X29_ITR. The purified hCOSMC and vector fragments were ligated in a final volume of 10 μL then used to transform 50 μL of competent DH5 alpha cells. The integrity and structure of the newly created plasmid was checked by restriction analysis, as described previously.

### Example 2: Generation of Cell Lines for Producing Recombinant Collagen 7

### Host Cell Lines and Cell Culture

A serum free cultivated cell bank (working cell bank, WCB) derived from the wild-type CHO-K1 cell line (ATCC, Cat. No.: CCL-61) was cultivated and maintained under serum free conditions in SFM4CHO medium (HyClone, Cat. No.: SH30548), supplemented with 8 mM L-Glutamine (PAA, Cat. No.: M411-004) and 1×HT supplement (Hypoxanthine/Thymidine supplement) (Invitrogen, Cat. No.: 41065). A research cell bank (RCB) was generated for transfections, by adding 5% CB5 (HyClone™, Cell Boost™ supplement (HyClone, Cat. No.: SH30865) to the media of the WCB. The cells were routinely seeded in a density of 2×10⁵ cells/mL. The transfectability and single cell plating capacity were tested and approved. These serum-free, suspension cell cultures ready for transfection are referred to as CHO-M cells.

CHO-M host cells were routinely cultivated in SFM4CHO medium supplemented with 8 mM L-Glutamine, 1×HT supplement and 5% CB5. Cells were maintained under agitation (120 rpm, 25 mm stroke) in a humidified incubator at 37° C. and 5% CO2. Prior to transfections, SFM4CHO medium supplemented with 8 mM L-Glutamine, 1×HT, and 5% CB5 was pre-warmed by plating 2 mL into one well of a 6-well plate and incubated at 37° C., 5% CO2.

### Expression Constructs/Plasmids Preparation

Constructs were made as described in detail in Example 1. All the plasmids were quantified and further verified by sequencing.

### Transfection (Pool #B1)

The SGE Tech 1 transfection system was used for cell transfection (SELEXIS Inc., USA). The collage 7 expression plasmid (carrying Puromycin resistance cassette) and two plasmids for prolyl-4-hydroxylase subunits A1 and B (P4HA1 and P4HB) were co-transfected into the CHO-M cells (Table 3) as follows. A GFP expression plasmid was used as control.

TABLE 3
Plasmids for SGEtech I transfection

| | Amount | Conc. DNA | |
|---|---|---|---|
| Plasmid | (μg/transfer) | (ng/μL) | Ratio |
| Puro BT+ SLX3631 Col7A, Pvul, | 1.233 | 1838 | 5 |
| 17984 bp | | | |
| pBSK ITR CGAPD hP4HA1 X29 | 0.139 | 4675 | 1 |
| ITR, 10220 bp | | | |
| pBSK ITR CGAPD hP4HB X29 | 0.138 | 3176 | 1 |
| ITR, 10148 bp | | | |

CHO-M cells were prepared shortly before the transfection procedure to maximize cell viability (96.0% viability) and transfection efficiency. Cells (5.1×10⁵ cells per microporation) were centrifuged (400×g, 5 min, room temperature) and washed in sterile 1×PBS. The cell pellets were gently resuspended in Resuspension Buffer R (MicroPorator Kit, MPK-1096) to a concentration of 1.7×10⁷ c/mL. A volume of 100 μL of cell suspension (per microporation) were immediately transferred to the DNA tubes and mixed carefully. Cell-DNA mixture was aspirated by a MicroPorator pipette (NanoEnTek Inc., Korea) and placed into the pipette station. After the microporation (1130V, 20 ms and 3 pulses), cells were transferred to the previously prepared 6-well plate and incubated overnight in a static, humidified incubator at 37° C. and 5% CO2. Transfection efficiency was controlled by using a GFP expressing vector in parallel (microscopic inspection conducted the following day showed normal transfection efficiency between 50-70%).

Six (6) days after the SGEtech I transfection, cells were transferred to spin tubes and selected in antibiotic containing medium (SFM4CHO medium with 8 mM L-Glutamine, 1×HT, 5% CB5 and 5 μg/mL of Puromycin (Sigma, Cat. No.: P-9620)). The transfected cells were cultured and passaged using SFM4CHO medium with 8 mM L-Glutamine, 1×HT, 5% CB5 and 2.5 μg/mL of Puromycin. One pool of transfected cells (Pool #B1) was used for the next transfection.

### Super Transfection of Pool #B1

Cells of Pool #B1 from the SGEtech I transfection were then additionally transfected (as Super Transfection) with the plasmids shown in Table 4. For the SuperTransfection, vectors carrying Hygromycin resistance were utilized. Cells (5.1×10⁵ cells/transfection) were combined with 4.5 μg linearized DNA sample in sterile reaction tubes. The same transfection protocol was used ((1130V, 20 ms and 3 pulses). Transfection efficiency was controlled by using GFP expressing vector (normal transfection efficiency between 50-70%).

TABLE 4
Plasmids for Super Transfection of Pool #B1

| | Amount | Conc. DNA | |
|---|---|---|---|
| Plasmid | (μg/transfer) | (ng/μL) | Ratio |
| Hygro BT+ SLX3631 Col7A, Pvul, | 1.228 | 3533 | 5 |
| 18422 bp | | | |
| pBSK ITR CGAPD hP4HA1 X29 | 0.136 | 4675 | 1 |
| ITR, 10220 bp | | | |
| pBSK ITR CGAPD hP4HB X29 | 0.135 | 3176 | 1 |
| ITR, 10148 bp | | | |

After Super Transfection, cells were expanded into 5 mL spin tubes. After culturing for ten days in SFM4CHO medium supplemented with 8 mM L-Glutamine, 1×HT and 5% CB5, including 2.5 g/mL of Puromycin, double selection was initiated by adding 250 μg/mL of Hygromycin (Invivogen, Cat. No.: ant-hm-5) to the medium. Six subsequent passages were performed before banking. One pool of transfected cells (Pool #B1STB) was used for the next transfection.

### Super Transfection of Pool #B1STB

Cells of Pool #B1STB were again transfected with the vectors expressing prolyl 4-hydroxylase A and B subunits (Table 5) and a mammalian expression vector U5-PB. For each microporation, 3.4×10⁵ cells and 3 μg linearized DNA were prepared in sterile reaction tubes, alongside a GFP control. The same transfection protocol was used (1130V; 20 ms; 3 pulses).

TABLE 5
Plasmids for Super Transfection of Pool #B1STB

| | Amount | Conc. DNA | |
|---|---|---|---|
| Plasmid | (μg/transfer) | (ng/μL) | Ratio |
| U5_PB, 6120 bp | 0.693 | 2027 | 1 |
| pBSK ITR CGAPD hP4HA1 X29 | 1.158 | 4675 | 1 |
| ITR, 10220 bp | | | |
| pBSK ITR CGAPD hP4HB X29 | 1.149 | 3176 | 1 |
| ITR, 10148 bp | | | |

Thirteen days after Super Transfection II, cells were expanded into 5 mL spin tubes in SFM4CHO medium supplemented with 8 mM L-Glutamine, 1×HT and 5% CB5, without antibiotics. On day 14, 250 μg/mL of Hygromycin and 2.5 μg/mL of Puromycin were added to the culture medium.

After 14 subsequent passages, the transfected cells were cryoconserved. One pool of the transfected cells (Pool #B1STBSTb) was further cultivated for another eight passages in SFM4CHO medium including 250 μg/mL of Hygromycin and 2.5 μg/mL of Puromycin for another super-transfection.

### Super Transfection of Pool #B1STBSTb

The cells from Pool #B1STBSTb were further transfected with the expression vector for hHSP47 (Table 6). For each microporation, 3.4×10⁵ cells and 3 μg of linearized DNA samples were prepared in sterile reaction tubes, alongside a GFP control. The same transfection protocol was used (1130V; 20 ms; 3 pulses).

TABLE 6
Plasmids for Super Transfection of Pool #B1STBSTb

| | Amount | Conc. DNA |
|---|---|---|
| Plasmid | (μg/transfer) | (ng/μL) |
| U5_PB, 6120 bp | 0.500 | 2027 |
| pBSK ITR BT+ SHSP47 X29 ITR, 10205 bp | 2.500 | 3629 |

Nine days after Super Transfection III, a medium exchange was performed and 200 μg/mL 2-Phospho-L-Ascorbic Acid (Sigma, Cat. No.: 49752) was added to the SFM4CHO culture medium already supplemented with 8 mM L-Glutamine, 1×HT, and 5% CB5, but no antibiotics. Five days later cells were expanded into 5 mL spin tubes in SFM4CHO medium, supplemented with 8 mM L-Glutamine, 1×HT and 5% CB5, including 250 μg/mL of Hygromycin and 2.5 μg/mL of Puromycin. Seven subsequent passages were performed in the medium including 250 μg/mL of Hygromycin and 2.5 μg/mL of Puromycin for further analysis.

One pool of the transfected cells (Pool #B1STBSTbSTh2) was further cultivated and expanded.

### Example 3. Screen of Monoclonal Cell Lines

### 3.1 Screen of Pool #B1STBSTb

Cells of Pool #B1STBSTb were cultured at the concentration of 100 cells/mL in semi-solid medium (2×SFM4CHO medium and methyltcellulose (CloneMatrix™, Genetix, Cat. No.: K8510) including 8 mM L-Glutamine, 1×HT and 5% CB5) for 11 days. One hundred eleven candidates were picked and transferred to 96-well plates in SFM4CHO medium supplemented with 8 mM L-Glutamine, 1×HT and 5% CB5. Within 7 days, the candidates were screened by dot-blot. 18 super transfected candidates were further picked and transferred to 24-well plates. Within another 7 days, the 24-well supernatants were analyzed by dot-blot and 12 super transfected candidates were transferred to 6-well plates.

All 12 highest expressing (based on dot-blot) super transfected candidates were expanded 7 days later to suspension cultivation in spin tubes (5 mL working volume) and after 5 subsequent passages into shake flasks (20 mL working volume) in SFM4CHO medium supplemented with 8 mM L-Glutamine, 1×HT and 5% CB5. All these expansions were performed without addition of antibiotics for selection.

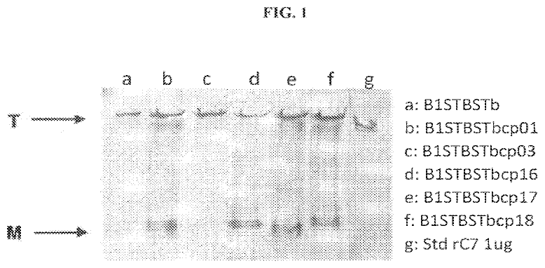

The candidates were banked, and the performance of each candidate was compared in batch cultivation at 32° C. Cell numbers and viability are shown in table 7. The protein expression was tested by western blot. FIG. 1 shows a gel image of the western blot of the Day 6 cultures of the B1STBSTb first round candidates.

TABLE 7
Pool B1STBSTb candidate results at 32° C.

| | Cell Conc. | Viability | Cell Conc. | Viability |
|---|---|---|---|---|
| | (c/mL) | (%) | (c/mL) | (%) |
| Clone candidate No. | Day 3 | Day 3 | Day 6 | Day 6 |
| B1STBSTb-cp01 | 8.39E+05 | 96.9 | 3.43E+06 | 96.3 |
| B1STBSTb-cp03 | 1.47E+06 | 98.2 | 6.78E+06 | 97.3 |
| B1STBSTb-cp16 | 1.19E+0 6 | 97.8 | 4.85E+06 | 96.0 |
| B1STBSTb-cp17 | 1.00E+06 | 96.9 | 1.35E+05 | 3.4 |
| B1STBSTb-cp18 | 7.61E+06 | 96.5 | 1.17E+07 | 96.0 |

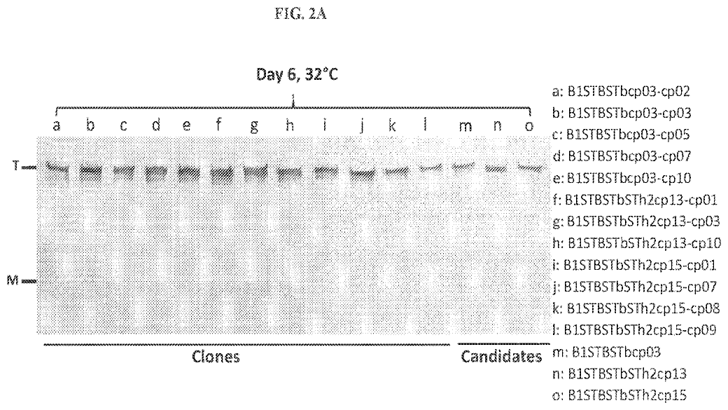

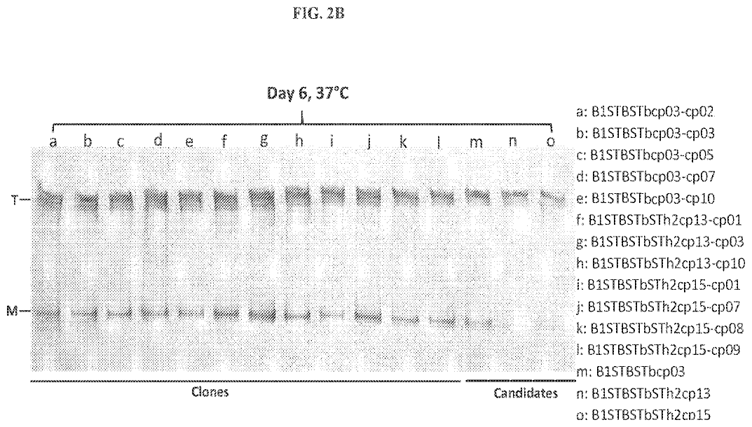

The candidate clone B1STBSTb-cp03 was further cultured and selected following the same process and 5 clones from B1STBSTb-cp03 were selected (Table 8). FIG. 2A and FIG. 2B show gel images of western blots of Day 6 cultures of clones from second-round selection at 32° C. and 37° C., respectively.

TABLE 8
Cell candidates from second round screen (32° C. and 37° C.)

| | 32° C. | | 37° C. | |
|---|---|---|---|---|
| | Cell Conc. | Viability | Cell Conc. | Viability |
| | (c/mL) | (%) | (c/mL) | (%) |
| Clone candidate No. | Day 6 | Day 6 | Day 6 | Day 6 |
| B1STBSTbcp03-cp02 | 4.16E+06 | 90.43 | 1.06E+07 | 91.61 |
| B1STBSTbcp03-cp03 | 2.22E+06 | 87.29 | 4.55E+06 | 86.05 |
| B1STBSTbcp03-cp05 | 4.48E+06 | 95.34 | 9.80E+06 | 95.97 |
| B1STBSTbcp03-cp07 | 6.18E+06 | 95.59 | 1.30E+07 | 94.90 |
| B1STBSTbcp03-cp10 | 4.85E+06 | 93.09 | 1.23E+07 | 95.91 |

### 3.2 Screen of Pool #B1STBSTbSTh2

Cells of Pool #B1STBSTbSTh2 were cultured at the concentration of 10⁰ cells/mL in semi-solid medium (2×SFM4CHO medium and methyltcellulose including 8 mM L-Glutamine, 1×HT and 5% CB5. Twenty-nine super transfected candidate clones were picked and transferred to 96-well plates in SFM4CHO medium supplemented with 8 mM L-Glutamine, 1×HT and 5% CB5 without antibiotics for selection.

Within 3 days, all 29 growing super transfected candidates were transferred to 6-well plates (1 mL cell suspension+2 mL fresh growth medium). Five days later, the 6-well supernatants were analyzed by western blot and 3 supertransfected candidates showing the highest expression were expanded to suspension cultivation in spin tubes (5 mL working volume). After five subsequent passages in SFM4CHO medium supplemented with 8 mM L-Glutamine, 1×HT and 5% CB5 without selection (plus 200 μg/mL 2-Phospho-L-Ascorbic Acid for the fourth passage), the cells were screened by western blot of the spin tube supernatants (5 mL working solution). The two super transfected candidates demonstrating the highest expression were then expended to suspension cultivation in shake flasks (20 mL working volume) in SFM4CHO medium supplemented with 8 mM L-glutamine, 1×HT and 5% CB % without antibiotics.

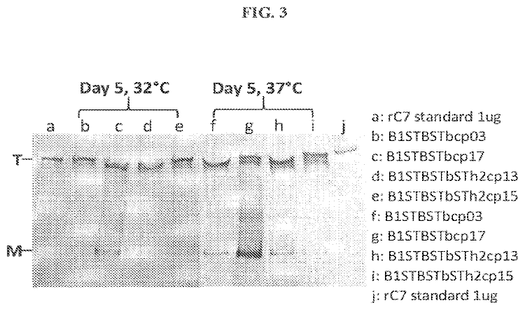

Two candidates, B1STBSTbSTh2-cp13 and B1STBSTbSTh2-cp15, were banked and performance was compared in batch cultivation at 32° C. and 37° C. Cell numbers and viability are shown in Table 8. FIG. 3 is a gel image of the western blot of Day 5 cultures of B1STBSTbSTh2cp13 and B1STBSTbSTh2cp15 at 32° C. and 37° C.

TABLE 9
B1STBSTbSTh2 first-round candidate results

| | 32° C. | | 37° C. | |
|---|---|---|---|---|
| | Cell Conc. | Viability | Cell Conc. | Viability |
| | (c/mL) | (%) | (c/mL) | (%) |
| Clone candidate No. | Day 5 | Day 5 | Day 5 | Day 5 |
| B1STBSTbSTh2cp13 | 2.89E+06 | 96.9 | 5.58E+06 | 86.0 |
| B1STBSTbSTh2cp15 | 3.60E+06 | 97.7 | 5.18E+06 | 89.3 |

The candidate clones B1STBSTbSTh2cp13 and B1STBSTbSTh2cp15, were further cultured and screened in semi-solid medium (2×SFM4CHO medium and methyltcellulose including 8 mM L-Glutamine, 1×HT and 5% CB5 (without antibiotics selection). Plated colonies were screened 12 days later using ClonePix cell colony picker system. 32 clones from B1STBSTbSTh2cp13 and 30 clones from B1STBSTbSTh2cp15 were picked and transferred to 96-well plates and later to 6-well plates in SFM4CHO medium supplemented with 8 mM L-Glutamine, 1×HT and 5% CB5 without antibiotics for selection. All picked cell clones were expanded three (3) days later into spin tubes (5 mL working volume). Based on analysis of the spin tube supernatants by western blot, 3 clones from B1STBSTbSTh2cp13 and 4 clones from B1STBSTbSTh2cp15 with the greatest expression were selected and expanded into shake flasks (20 mL working volume) in SFM4CHO medium supplemented with 8 mM L-Glutamine, 1×HT and 5% CB5 (without antibiotics for selection).

All the cell lines (listed in Table 10) were banked and performance of these cells was compared in batch cultivations at 32° C. and 37° C. Cell numbers and viability are shown in Table 10. FIG. 2A and FIG. 2B show gel images of western blots of Day 6 cultures of clones from second-round selection at 32° C. and 37° C., respectively.

TABLE 10
Cell candidates from second round screen (32° C. and 37° C.)

| | 32° C. | | 37° C. | |
|---|---|---|---|---|
| | Cell Conc. | Viability | Cell Conc. | Viability |
| | (c/mL) | (%) | (c/mL) | (%) |
| Clone candidate No. | Day 6 | Day 6 | Day 6 | Day 6 |
| B1STBSTbSTh2cp13- | 3.03E+06 | 92.07 | 3.69E+06 | 83.15 |
| cp01 | | | | |
| B1STBSTbSTh2cp13- | 1.32E+06 | 90.90 | 4.12E+06 | 84.11 |
| cp03 | | | | |
| B1STBSTbSTh2cp13- | 1.64E+06 | 88.78 | 2.93E+06 | 73.91 |
| cp10 | | | | |
| B1STBSTbSTh2cp15- | 1.98E+06 | 93.26 | 3.75E+06 | 86.00 |
| cp01 | | | | |
| B1STBSTbSTh2cp15- | 2.19E+06 | 91.79 | 4.57E+06 | 90.80 |
| cp07 | | | | |
| B1STBSTbSTh2cp15- | 2.50E+06 | 87.18 | 4.70E+06 | 88.49 |
| cp08 | | | | |
| B1STBSTbSTh2cp15- | 2.52E+06 | 92.58 | 5.36E+06 | 92.53 |
| cp09 | | | | |

All cell lines were cryoconserved using 10% DMSO (Sigma, Cat. No.: D-2650), 45% conditioned medium and 45% fresh SFM4CHO medium supplemented with 8 mM L-Glutamine, 1×HT and 5% CB5, at 6×10⁶ cells/vial Vials were stored in the cryobox (Nunc) for 24 hours at −80° C. and then transferred to a Liquid Nitrogen System with restricted access.

Frozen cells were tested and confirmed for absence of mycoplasma using VenoR Gem mycoplasma detection kit (Minerva Biolabs, Cat. No.: 11-1100), sterility according to the manufacturer's protocol (Heipha, Caso-Bouillon TSB, No.: 3080r).

The stability of the cells including viability at thaw, cell growth and cDNA sequencing and post-thaw functionality testing is monitored at specified time intervals.

### Example 4: Validation of Cell Clone #B1STBSTbcp03 (MCB) for rCol7 Production

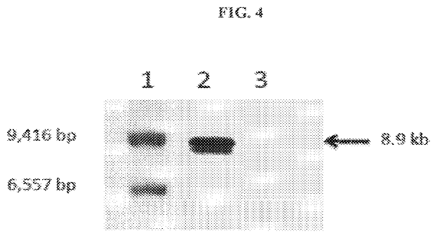

Collagen 7 produced by the cell clone #B1STBSTbcp03 (also referred as MCB cells) was extensively tested following ICH (International Council for Harmonisation of Technical Requirements for Pharmaceuticals for Human Use) safety guidelines. The identity of the rCol7 from MCB was analyzed by Southern blot and rCol7 cDNA sequencing. The expected 8.9 kb rCol7 fragment is present in the MCB, but not in the host CHO cells, confirming the presence of the rCol7 coding region in the MCB (FIG. 4). The nucleotide sequence of the cDNA isolated from the rCol7 MCB was confirmed to encode rCol7 by sequencing analysis.

The sequencing results showed a sequence heterogeneity at nucleotide position 3097 (counted from the ATG start codon) of rCol7 from MCB. The nucleotide residue at 3097 is a mixture of natural G nucleotide (as in GenBank Accession #NM_000094; SEQ ID NO.: 2) and T nucleotide which was the predominant nucleotide observed at this position (20.2% G and 79.8% T), resulting in heterogeneous rCol7 polypeptides having the amino acid residue aspartic acid (D) (encoded by GAC) and tyrosine (Y) (encoded by TAC) at codon 1033 of collagen 7 polypeptide. Since the nucleotide sequence of two rCol7 expressing constructs (Puro_BT+SLX3631_Col7A and Hygro_BT+SLX3631_Col7A) was confirmed to correctly encode aspartic acid (D) at position 1033, it is concluded that the observed heterogeneity was introduced during the cell line development process leading to the generation of the MCB.

These results indicate that the MCB is biclonal, or a mixture of two related clones carrying either the G3097 or the T3097 recombinant collagen 7 sequences. The ratio of the G:T sequences and, consequently, the ratio of the two clones, is approximately 1:4. Due to the nucleic acid sequence heterogeneity, the recombinant human collagen 7 composition produced by the rCol7 MCB is also heterogeneous, comprising a mixture of D1033 and Y1033, with the Y1033 variant comprising approximately 80% to 90% of the material.

Further evaluation was performed to test the functionality of rCol7 materials produced by the MCB. The potential impact of D1033Y heterogeneity on rCol7 structure and function was evaluated through a combination of molecular modeling techniques. The results confirmed the expected physicochemical properties (e.g., molecular weight, proline hydroxylation, primary and secondary structure). By established homology models (in silico), the heterogeneity at position 1033 is not likely to affect the β sandwich folding of the fibronectin type III repeat 9 (FNIII domain FNIII R9) of collagen VII.

To evaluate the impact of the heterogeneity at position 1033 on the functionality of collagen 7, several important attributes were measured for MCB-derived rCol7 composition (e.g., MCB rCol7) and compared against reference collagen 7 (from human fibroblasts). These analyses included identity assessment by Western blot in reduced conditions, evaluation of multimerization state by size exclusion-HPLC (SE-HPLC), hydroxyproline occupancy by peptide mapping LC/MS, biophysical assessment of domain integrity through differential scanning calorimetry (DSC), binding partner affinity assessment through a laminin-332 binding assay, and assessment of wound healing through an IncuCyte wound healing method.

DSC (differential scanning calorimetry) was employed to assess the domain integrity of MCB rCOL7. The DSC analyses yielded comparable results for the reference collagen 7 and current MCB-derived rCol7 composition, with very similar domain/sub-domain thermal transitions being observed. The collagenous domain of collagen 7 undergoes the earliest thermal transition (Tm) at approximately 45° C. in both materials and the non-collagenous domain (NC1) melting transition occurs at the temperature of approximately 68° C. The intermediate thermal transition within both materials is thought to be a sub-domain of the collagenous domain with a similar transition in both materials of 49° C. to 50° C.

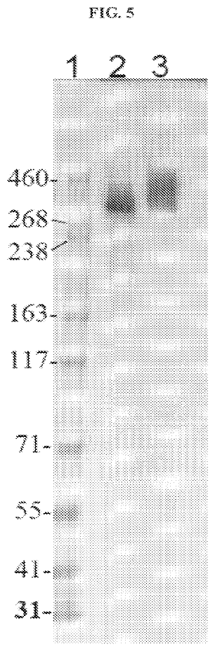

The Western blot analysis indicated that the rCol7 product identity was comparable between the reference collagen 7 and the current MCB-derived rCol7 composition, in terms of the primary banding pattern observed (FIG. 5).

The MCB rCol7 samples were denatured, reduced, and the cysteines alkylated through the use of iodoacetic acid and then digested with trypsin at 37° C. for 14 hours. The digested protein was deglycosylated by PNGase F. The peptide mixture was analyzed using peptide mapping LC/MS. The results indicated the integrity of rCol7 primary sequence. The level of hydroxyproline in MCB rCol7 was estimated by the percentage of hydroxyproline observed within the T724 indicator peptide (VVGAPGVPGAPGER (Bulleid et al., The EMBO Journal, 1997, Vol. 16 (22): 6694-6701). The peptide mapping LC/MS results indicate a similar level of hydroxyproline occupancy within the T274 indicator peptide.

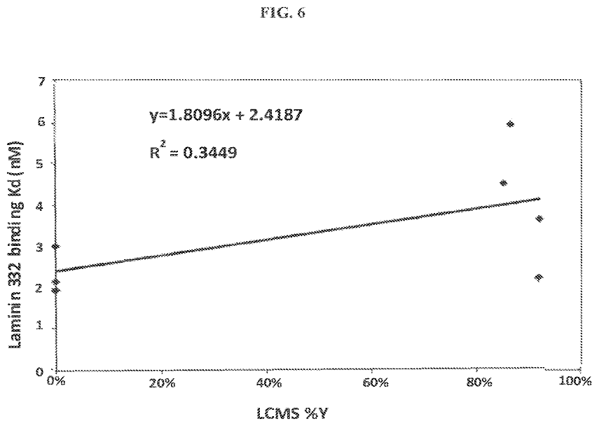

Residue 1033 is located in the ninth fibronectin type III-like repeat of collagen 7, which has been mapped within the laminin-332 binding site (Chen et al., J Invest Dermatol., 1999, Vol. 112(2): 177-183). An assessment to determine the potential correlation between the laminin-332 binding affinity and the percentage of the Y1033 variant in MCB-derived rCol7 indicates that the binding characteristics, such as maximal binding level and dissociation constant (Kd) for the reference collagen 7 and MCB rCol7 were similar (FIG. 6). Thus, the heterogeneity present at amino acid position 1033 does not appear to impact the laminin-332 binding of MCB-derived rCol7 composition.

Binding assessment was also carried out with fibronectin, an additional binding partner of collagen 7 which is not present in the BMZ.

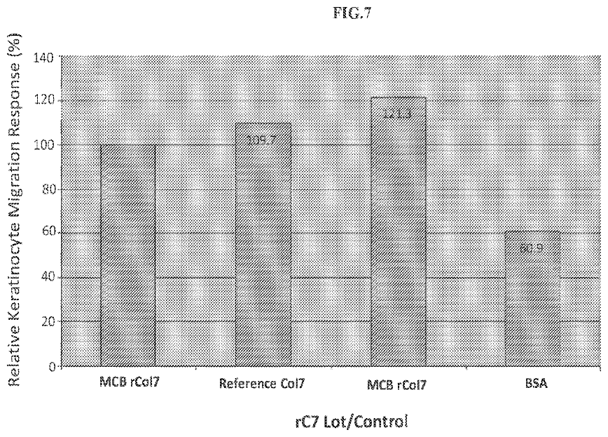

An in vitro wound healing bioassay for the reference collagen 7 and the MCB rCol7 was performed. The test results were similar between these materials, indicating that the heterogeneity present at position 1033 does not adversely impact the biological function measured by this assay (FIG. 7).

Taken together, the physicochemical and functional testing of the reference collagen 7 and MCB-derived rCol7 compositions demonstrated similarity across a number of important attributes, indicating a lack of significant impact of tyrosine, instead of aspartic acid at position 1033 in collagen 7.

### Example 5: Validation of Clones #B1STBSTbSThcp13-01 and #B1STBSTbSThcp13-03

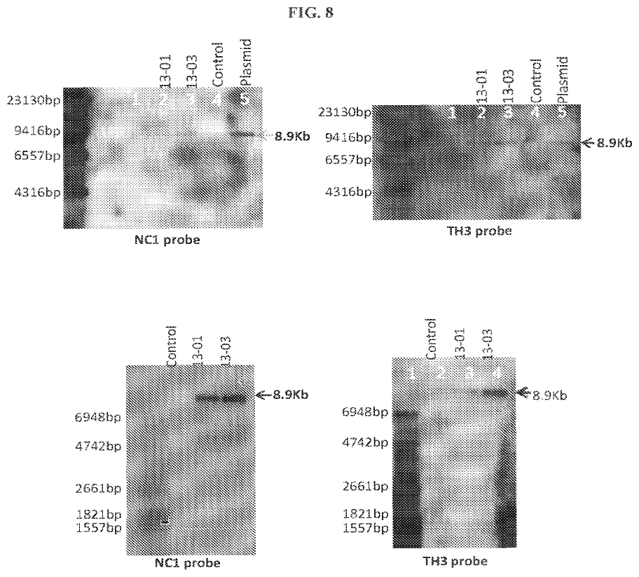

Cell clones #B1STBSTbSThcp13-01 and B1STBSTbSThcp13-03 were originated from two rounds of cloning that meets the industrial standard to ensure high probability of monoclonality (i.e., single cell origin). DNA sequence analysis of isolated genomic DNA (gDNA) and cDNA from both clones confirmed the presence of a single nucleic acid sequence that matches the wild type collagen 7 sequence. This result confirms that both cell clones are monoclonal (i.e., derived from a single cell progenitor) and that the rCol7 transgene encodes native collagen 7 for both clones as defined in the reference sequence (human collagen 7, GenBank Accession #NM_000094; SEQ ID NO.: 2). South blot analysis confirmed the presence of 8.9 Kb fragment of rCol7 indicating the rCol7 coding sequence is intact in both clones (FIG. 8). Cultured cells from the cell bank a year later show no discrepancies from the reference Col7 sequence. The intact transgene sequences and transcript (mRNA) are present in both clones.

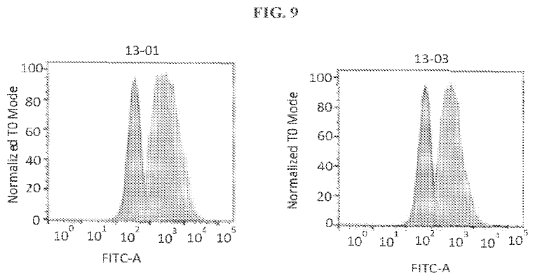

Cells from both clones are stable as indicated by the following stability test. Cells were cultured for at least 30 generations in the absence or presence of selection. Collagen 7 production was tested by intracellular staining for collagen 7 and ELISA test (Table 11 and FIG. 9). In the absence of selection, the productivity is declined (Table 11), while the productivity is maintained in the presence of selection for at least 30 generations (Table 11). The intracellular staining for collagen 7 confirms that there are no additional subpopulations observed, indicating that both cell lines are stable (FIG. 9)

TABLE 11
ELISA for cell productivity
Specific productivity (pg/cell/day)

| Generations | B1STBSTbSThcp13-01 | B1STBSTbSThcp13-03 |
|---|---|---|
| 0 | 1.1 | 1.5 ± 0.1 |
| 30 (+) selection | 1.2 ± 0.1 | 1.5 ± 0.1 |
| 30 (−) selection | 0.9 ± 0.1 | 0.8 ± 0.1 |

It was further determined that by sequencing no detectable mutant peptides were found in rCol7 compositions from B1STBSTbSThcp13-01 and B1STBSTbSThcp13-03. Biophysical features of rCol7 materials from B1STBSTbSThcp13-01 and B1STBSTbSThcp13-03 have the same unfolding temperature profiles (35° C. to 55° C.) by DSC mapping.

### Example 6: Cell Culture Conditions and Productivity of Process Scale-Up

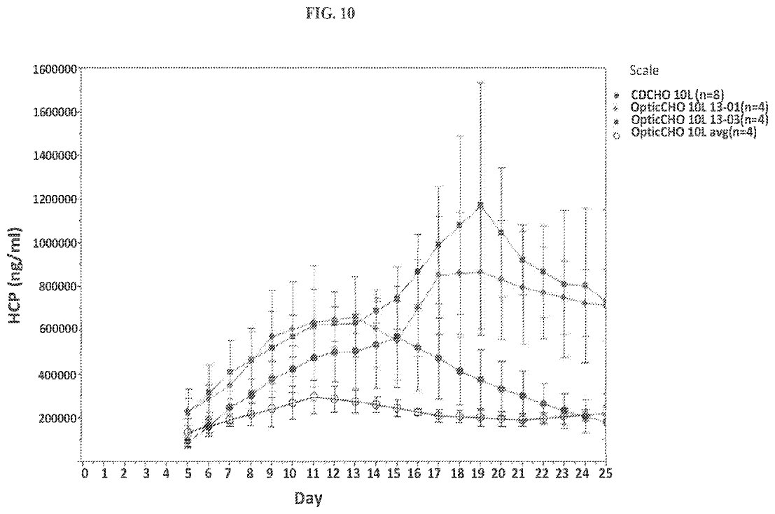

Successful process scale-up depends on determining, measuring, and monitoring critical scale-independent process parameters, then designing and operating equipment appropriately to deliver those same parameters at large scale. Bioreactor conditions including cell culture medium for cell expansion was optimized for process scale-up. The basal cell culture media, CD OptiCHO™ medium (Thermo Fisher) with cell boost 5, 100×HT and ascorbic acid was used. Cell growth was maintained at 30E6 vc/mL in 10 liter CD OptiCHO™ medium based culture, and the viability of clone B1STBSTbSThcp13-01 was consistently above 85% over 25 days of culture. The clone screening data show that the productivity of both clones B1STBSTbSThcp13-01 and B1STBSTbSThcp13-03 is higher than clone #B1STBSTbcp03 (Table 12) by ELISA. HCP (Host cellular protein) quantifications for clones B1STBSTbSThcp13-01 and B1STBSTbSThcp13-03 are also higher than that of the MCB clone (FIG. 10).

TABLE 12
Collagen 7 Productivity

| Media | Clone | 10 L bioreactor productivity |
|---|---|---|
| CD CHO | MCB (B1STBSTbcp03) | 12.5 mg/L/day |
| OptiCHO | MCB (B1STBSTbcp03) | 15 mg/L/day |
| OptiCHO | B1STBSTbSThcp13-01 | 34 mg/L/day |
| OptiCHO | B1STBSTbSThcp13-03 | 32 mg/L/day |

Three candidate clones were tested for scalability challenges. Cells were cultured in 400SW bioreactor for process scale-up. The oxygen update rate (OUR) was 1.59E-10 (mmol O2/hr*vc and 3.09E-10 (mmol O2/hr*vc) for B1STBSTbcp03 and B1STBSTbSThcp13-01, respectively. B1STBSTbSThcp13-03 clone has 20% less biomass and 20% more O2 flow rate. Packing was observed at the entrance and both B1STBSTbSThcp13-01 and B1STBSTbSThcp13-03 clones have similar packed cell volume at peak (20-25%). B1STBSTbcp03 cells have a uniform packing profile and about 20% packed cell volume (peak). The preliminary data support that B1STBSTbSThcp13-01 is preferred for process scale-up.

### Example 7: Downstream Purification Process Using Process Scale-Down for Evaluation

In order to deliver a fast, efficient, and reliable production process, the downstream purification processing was tested using scaled-down laboratory models. In the purification of recombinant therapeutic collagen 7 proteins, the downstream process was evaluated and optimized for increased protein purity and yield. The process incudes steps of:
1. Harvesting cell culture materials (UPB); in this study, a mixed and pooled culture material from each clone was used;
2. Inactivating viruses in cell culture materials (UPB) using UV-C and chemicals (e.g., Triton);
3. Filtering processed UPB through Capto™ core flow chromatography and sepharose chromatography;
4. Virus filtration;
5. Performing a final UF (ultrafiltraion) and DF (Dialfiltration) step using membrane materials. In this test study, a two-stage 100 KD UF/DF (e.g., 100 KD C-screen PES (polyethersulfone) membrane) was used. Alternatively, a single stage UF/DF (e.g., 30 KD A-screen PES membrane) was used.
6. Filtering the final products through a 0.2 μm membrane.

TABLE 13
Downstream purification yields

| | Eng | GMP | GMP | GMP | B1STBST | B1STBSTbSThc | B1STBSTbSThc |
|---|---|---|---|---|---|---|---|
| Yield | run | 1 | 2 | 3 | bcp03 | p13-01 | p13-03 |
| Scaled-down | RDD230 | RDD223A | RDD223B | RDD228 | RDD229C | RDD229A | RDD229B |
| Run # | | | | | | | |
| Total | 4,430,647 | 6,850,255 | 6,763,275 | 5,722,765 | 2,096,560~ | 13,343,216 | 14,981,835 |
| Bioreactor | | | | | 7,514,280 | | |
| HCP (ng/mL) | | | | | | | |
| UPB Titer | 88,000 | 59,990 | 87,000 | 72,000 | 143,419 | 208,000 | 134,000 |
| (ng/mL) | | | | | | | |
| UPB HCP | 40,104 | 52,555 | 49,243 | 37,825 | 170,160 | 339,140 | 406,110 |
| (ng/mL) | | | | | | | |
| UPB | 2.19 | 1.14 | 1.77 | 1.90 | 0.84 | 0.61 | 0.33 |
| rC7:HCP | | | | | | | |
| HCP ppm | 455,727 | 876,063 | 566,011 | 525,347 | 1,186,454 | 1,630,481 | 3,030,672 |
| Final UF/DF | 22% | 54% | 48% | 61% | 77% | 70% | 40% |
| step yield | | | | | | | |
| Overall | 20% | 12% | 27% | 18% | 35% | 24% | 35% |
| process | | | | | | | |

The UPB from clones B1STBSTbSThcp13-01 and B1STBSTbSThcp13-03 have significantly higher titer (208,000 ng/mL for clone 13-01) and 134,000 ng/ml for clone 13-03) as compared to ENG (88,000 ng/mL) and GMP (Table 13). Similarly, HCP from cell clones is significantly higher as compared to ENG and GMP. HCP levels in drug substance in each clone are comparable and below specification.

The test downstream purification yields are comparable using single stage 30 KD UF/DF or two-stage 100 KD UF/DF process. The current downstream process has comparable yield for all three clones but B1STBSTbSThcp13-01 and B1STBSTbcp03 have higher final UF/DF yield (Table 13).

The drug substance quality attributes from the scaled-down process were evaluated as shown in Table 14. Other attributes such appearance, bioburden, endotoxin, osmolality and Ph of drug substances from cell clones are comparable to reference standards. The analytical results from at scale runs and 400SW bioreactor are similar (data not shown).

TABLE 14
Drug substance quality attributes from scaled-down process

| Drug substance | B1STBSTbcp03 | B1STBSTbSThcp13-01 | B1STBSTbSThcp13-03 |
|---|---|---|---|
| attributes | (10 L scale-down UPB) | (10 L scale-down UPB) | (10 L scale-down) |
| Protein concentration | 0.77 | 0.49 | 0.43 |
| (A280) by Solo VPE | | | |
| HCP (ng/mg) | 900 | 592 | 1281 |
| Size by SE-HPLC | 8.2 | 4.4 | 5.1 |
| (HMW: ≤25.0%) | | | |
| Size by SE-HPLC | 74.2 | 78.6 | 62.7 |
| (Main Peak: ≥40.0%) | | | |
| Size by SE-HPLC | 17.6 | 17.0 | 32.2 |
| (LMW: Report result) | | | |
| Potency by Fibronectin | >100% | 56% | 81% |
| Binding | | | |
| Potency by IncuCyte | 90% | 116% | 113% |
| Wound healing | | | |
| Product Heterogeneity: | / | <LOD | <LOD |
| % Y at position 1033 | | | |
| (peptide map) | | | |
| Hydroxylation: % | / | 78.4 | 81.8 |
| Occup. T274 indicateo | | | |
| (Peptide map) | | | |
| Hydroxylation: % | / | 47.1 | 52.9 |
| Triply occupied T274 | | | |
| indicateo (Peptide | | | |
| map) | | | |
| rCol7 content in drug | / | 1.18 | 1.38 |
| substance (Average % | | | |
| of CHO Col 7 to | | | |
| rCol7) | | | |

A higher total HCP from bioreactor and UPB was measured for both clones #B1STBSTbSThcp13-01 and B1STBSTbSThcp13-03 by ELISA. The HCP content in drug substance is comparable between the scale-down runs of three cell clones by ELISA Method (Table 14), although increased CHO C7 content was measured for drug substance from B1STBSTbSThcp13-01 and B1STBSTbSThcp13-03 cell lines by MS Method (Table 15).

TABLE 15
CHO Col7 content in drug substances from cell lines

| | Col 7 | | | | | |
|---|---|---|---|---|---|---|
| Drug | control | B1STBSTbcp03 | GMP1 | B1STBSTbcp03 | | |
| substance | (Fibroblast) | (PUR034M01) | (RDD223A) | (10L) | B1STBSTbSThcp13-01 | B1STBSTbSThcp13-03 |
| Average | 0 | 1.67 | 0.10 | / | 1.18 | 1.38 |
| (% CHO | | | | | | |
| Col7 to | | | | | | |
| rCol7) | | | | | | |

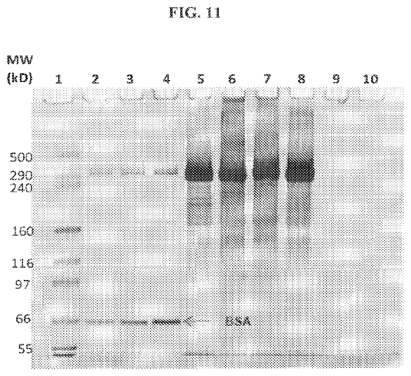

These results suggest that the productivity of #B1STBSTbSThcp13-01 can be >20 mg/L/day. The rCol7 produced from the cell clones contains natural collagen 7 (FIG. 11). No detectable mutant polypeptides from rCol7 from B1STBSTbSThcp13-01 and B1STBSTbSThcp13-03 were found by sequencing recombinant protein. The cell line is stable and suitable for scaled-up cell culture. The overall downstream purification yield and drug substance attributes are comparable to standard refence materials. Other functions of the drug substance from B1STBSTbSThcp13-01 cell line (e.g., potency by IncuCyte wound healing assay and fibronectin binding) are comparable to refence standard (Table 14). The detailed results are discussed in the following examples.

### Example 8: RCol7 Sequence Validation from Selected Monoclonal Host Cells

Total RNA contents were extracted from engineered recombinant host cell clones and quantified following standard procedures. The quality of the RNA extracts was also analyzed. The target transcripts (sequences subject to sequence validation) were enriched using RT-PCR (reverse-transcriptase mediated PCR) and RACE-PCR (Rapid Amplification of cDNA Ends-PCR).

Amplification products (amplicons) were constructed to generate DNA libraries for each amplicon. Each DNA library was uniquely barcoded for sampling tracking and identification. The average of DNA fragment length of each DNA library was assessed upon completion of DNA library construction (Agilent 2100 Bioanalyzer High Sensitivity DNA Kit).

The final DNA libraries were quantified using SYBR-QPCR (SYBR Green-based quantitative polymerase chain reaction) and analyzed to determine the DNA concentration. The final DNA libraries were denatured, diluted, combined together, and sequenced by NGS (Next Generation Sequencing) (Illumina® MiSeq® NGS platform).

Sequence data were analyzed by mapping and aligning of the RT-PCT and RACE-PCR data sets against the reference rCol7 sequence (SEQ ID NO.: 25; collagen 7 insertion in the rCol7 constructs).

Preliminary RACE-PCR mapping data indicated that approximately 80% of the read population mapped to the reference genome and the alignment yielded 100% reference coverage at 100% similarity with no reportable variants detected.

1. A production system for producing a collagen 7 composition, comprising host cells modified to express human recombinant collagen 7 (rCol7) and/or functional variants thereof having at least 90% sequence identity to a wild-type human collagen 7,
wherein the host cells are modified to comprise:
(a) a vector comprising at least one exogenous polynucleotide for expressing a recombinant human collagen 7 alpha chain polypeptide, and/or a functional variant thereof having at least 90% sequence identity to a wild-type human collagen 7 alpha chain polypeptide;
(b) a vector comprising an exogenous polynucleotide for expressing an alpha polypeptide of prolyl 4-hydroxylase, or a functional variant thereof having at least 90% sequence identity to a wild-type alpha polypeptide of prolyl 4-hydroxylase; and
(c) a vector comprising an exogenous polynucleotide for expressing a beta polypeptide of prolyl 4-hydroxylase, or a functional variant thereof having at least 90% sequence identity to a wild-type beta polypeptide of prolyl 4-hydroxylase, wherein the vector is different from the vector comprising an exogenous polynucleotide for expressing an alpha polypeptide of prolyl 4-hydroxylase, or the functional variant thereof,
wherein the host cells are mammalian cells, and wherein the collagen 7 composition comprises human rCol7, functional variants of human collagen 7 having at least 90% sequence identity to a wild-type human collagen 7, or the combination thereof.

2. The production system of claim 1, wherein the host cells are further modified to comprise:
(d) a vector comprising a second exogenous polynucleotide for expressing a recombinant human collagen 7 alpha-chain polypeptide, or a functional variant thereof having at least 90% sequence identity to a wild-type human collagen 7 alpha chain polypeptide, wherein the second exogenous polynucleotide comprises:
(i) the same nucleic acid sequence as that of the first exogenous polynucleotide encoding a recombinant human collagen 7 alpha-chain polypeptide, or a functional variant thereof having at least 90% sequence identity to a wild-type human collagen 7 alpha chain polypeptide; or
(ii) a different nucleic acid sequence from that of the first exogenous polynucleotide encoding a recombinant human collagen 7 alpha-chain polypeptide, or a functional variant thereof having at least 90% sequence identity to a wild-type human collagen 7 alpha chain polypeptide.

3. The production system of claim 2, wherein the first exogenous polynucleotide and the second exogenous polynucleotide comprise different selection markers.

4. The production system of claim 2, wherein:
(a) the first exogenous polynucleotide for expressing the recombinant human collagen 7 alpha chain polypeptide, or the functional variant thereof, comprises a nucleic acid sequence of SEQ ID NO.: 25;
(b) the exogenous polynucleotide for expressing the alpha polypeptide of prolyl 4-hydroxylase, or the functional variant thereof, comprises a nucleic acid sequence of SEQ ID NO.: 28;
(c) the exogenous polynucleotide for expressing the beta polypeptide of prolyl 4-hydroxylase, or the functional variant thereof, comprises a nucleic acid sequence of SEQ ID NO.: 30; and
(d) the second exogenous polynucleotide for expressing the recombinant human collagen 7 alpha-chain polypeptide, or the functional variant thereof, comprises a nucleic acid sequence of SEQ ID NO.: 25.

5. The production system of claim 1, wherein the alpha polypeptide of prolyl 4-hydroxylase is an alpha-I, an alpha-2, or an alpha-3 polypeptide.

6. The production system of claim 5, wherein the alpha polypeptide of prolyl 4-hydroxylase is the alpha-I polypeptide.

7. The production system of claim 1, wherein the host cells are mammalian cells selected from the group consisting of fibroblasts, keratinocytes, CHO cells, HEK293 cells, C127 cells, VERO cells, BHK cells, HeLa cells, COS cells and MDCK cells; or progenies thereof.

8. The production system of claim 7, wherein the modified host cells are cultured in a serum-free medium.

9. The production system of claim 7, wherein the host cells are CHO cells or progeny of CHO cells.

10. The production system of claim 1, further comprising:
(d) a vector comprising an exogenous polynucleotide for expressing a heat shock protein 47, or a functional variant thereof having at least 90% sequence identity to a wild-type heat shock protein 47.

11. The production system of claim 10, wherein the exogenous polynucleotide for expressing the heat shock protein 47, or the functional variant thereof, comprises a nucleic acid sequence of SEQ ID NO.: 32.

12. The production system of claim 4, further comprising a vector comprising an exogenous polynucleotide for expressing a heat shock protein 47, or a functional variant thereof having at least 90% sequence identity to a wild-type heat shock protein 47, wherein the exogenous polynucleotide for expressing the heat shock protein 47, or the functional variant thereof, comprises a nucleic acid sequence of SEQ ID NO.: 32.

13. The production system of claim 3, further comprising a vector comprising an exogenous polynucleotide for expressing a heat shock protein 47, or a functional variant thereof having at least 90% sequence identity to a wild-type heat shock protein 47, wherein the exogenous polynucleotide for expressing the heat shock protein 47 or the functional variant thereof, comprises a nucleic acid sequence of SEQ ID NO.: 32.
